SEQUENCE LISTING (20 sequences)

SEQ ID NO: 1 - protein, 239 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 2 - DNA, 720 bp, Homo sapiens
atggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtga

SEQ ID NO: 3 - protein, 233 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 4 - DNA, 702 bp, Homo sapiens
atgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatga

SEQ ID NO: 5 - protein, 198 aa, Homo sapiens
MAKQPSDVSSECDREGRQLQPAERPPQLRPGAPTSLQTEPQGNPEGNHGGEGDSCPHGSPQGPLAPPASPGPFATRSPLFIFMRRSSLLSRSSSGYFSFDTDRSPAPMSCDKSTQTPSPPCQAFNHYLSAMASMRQAEPADMRPEIWIAQELRRIGDEFNAYYARRVFLNNYQAAEDHPRMVILRLLRYIVRLVWRMH

SEQ ID NO: 6 - DNA, 597 bp, Homo sapiens
atggcaaagcaaccttctgatgtaagttctgagtgtgaccgagaaggtagacaattgcagcctgcggagaggcctccccagctcagacctggggcccctacctccctacagacagagccacaaggtaatcctgaaggcaatcacggaggtgaaggggacagctgcccccacggcagccctcagggcccgctggccccacctgccagccctggcccttttgctaccagatccccgcttttcatctttatgagaagatcctccctgctgtctcgatcctccagtgggtatttctcttttgacacagacaggagcccagcacccatgagttgtgacaaatcaacacaaaccccaagtcctccttgccaggccttcaaccactatctcagtgcaatggcttccatgaggcaggctgaacctgcagatatgcgcccagagatatggatcgcccaagagttgcggcgtattggagacgagtttaacgcttactatgcaaggagggtatttttgaataattaccaagcagccgaagaccacccacgaatggttatcttacgactgttacgttacattgtccgcctggtgtggagaatgcattga

SEQ ID NO: 7 - protein, 168 aa, Homo sapiens
MFQIPEFEPSEQEDSSSAERGLGPSPAGDGPSGSGKHHRQAPGLLWDASHQQEQPTSSSHHGGAGAVEIRSRHSSYPAGTEDDEGMGEEPSPFRGRSRSAPPNLWAAQRYGRELRRMSDEFVDSFKKGLPRPKSAGTATQMRQSSSWTRVFQSWWDRNLGRGSSAPSQ

SEQ ID NO: 8 - DNA, 507 bp, Homo sapiens
atgttccagatcccagagtttgagccgagtgagcaggaagactccagctctgcagagaggggcctgggccccagccccgcaggggacgggccctcaggctccggcaagcatcatcgccaggccccaggcctcctgtgggacgccagtcaccagcaggagcagccaaccagcagcagccatcatggaggcgctggggctgtggagatccggagtcgccacagctcctaccccgcggggacggaggacgacgaagggatgggggaggagcccagcccctttcggggccgctcgcgctcggcgccccccaacctctgggcagcacagcgctatggccgcgagctccggaggatgagtgacgagtttgtggactcctttaagaagggacttcctcgcccgaagagcgcgggcacagcaacgcagatgcggcaaagctccagctggacgcgagtcttccagtcctggtgggatcggaacttgggcaggggaagctccgccccctcccagtga

SEQ ID NO: 9 - protein, 261 aa, Homo sapiens
MKFGMGSAQACPCQVPRAASTTWVPCQICGPRERHGPRTPGGQLPGARRGPGPRRPAPLPARPPGALGSVLRPLRARPGCRPRRPHPAARCLPLRPHRPTRRHRRPGGFPLAWGSPQPAPRPAPGRSSALALAGGAAPGVARAQRPGGSGGRSHPGGPGSPRGGGTVGPGDRGPAAADGGRPQRTVRAAETRGAAAAPPLTLEGPVQSHHGTPALTQGPQSPRDGAQLGACTRPVDVRDSGGRPLPPPDTLASAGDFLCTM

SEQ ID NO: 10 - DNA, 786 bp, Homo sapiens
atgaaatttggcatggggtctgcccaggcatgtccatgccaggtgcccagggctgcttccacgacgtgggtcccctgccagatttgtggccccagggagcgccatggcccgcgcacgccaggagggcagctccccggagcccgtagagggcctggcccgcgacggcccgcgccccttcccgctcggccgcctggtgccctcggcagtgtcctgcggcctctgcgagcccggcctggctgccgcccccgccgcccccaccctgctgcccgctgcctacctctgcgcccccaccgccccacccgccgtcaccgccgccctggggggttcccgctggcctgggggtccccgcagccggccccgaggcccgcgcccggacggtcctcagccctcgctctcgctggcggagcagcacctggagtcgcccgtgcccagcgccccgggggctctggcgggcggtcccacccaggcggccccgggagtccgcggggaggaggaacagtgggcccgggagatcggggcccagctgcggcggatggcggacgacctcaacgcacagtacgagcggcggagacaagaggagcagcagcggcaccgcccctcaccctggagggtcctgtacaatctcatcatgggactcctgcccttacccaggggccacagagcccccgagatggagcccaattaggtgcctgcacccgcccggtggacgtcagggactcggggggcaggcccctcccacctcctgacaccctggccagcgcgggggactttctctgcaccatgtag

SEQ ID NO: 11 - protein, 197 aa, Homo sapiens
MFGLKRNAVIGLNLYCGGAGLGAGSGGATRPGGRLLATGAKDTKPMGRSGATSRKALETLRRVGDGVQRNHETAFQGMLRKLDIKNEDDVKSLSRVMIHVFSDGVTNWGRIVTLISFGAFVAKHLKTINQESCIEPLAESITDVLVRTKRDWLVKQRGWDGFVEFFHVEDLEGGIRNVLLAFAGVAGVGAGLAYLIR

SEQ ID NO: 12 - DNA, 594 bp, Homo sapiens
atgtttggcctcaaaagaaacgcggtaatcggactcaacctctactgtgggggggccggcttgggggccggcagcggcggcgccacccgcccgggagggcgacttttggccaccggcgccaaggacacaaagccaatgggcaggtctggggccaccagcaggaaggcgctggagaccttacgacgggttggggatggcgtgcagcgcaaccacgagacggccttccaaggcatgcttcggaaactggacatcaaaaacgaagacgatgtgaaatcgttgtctcgagtgatgatccatgttttcagcgacggcgtaacaaactggggcaggattgtgactctcatttcttttggtgcctttgtggctaaacacttgaagaccataaaccaagaaagctgcatcgaaccattagcagaaagtatcacagacgttctcgtaaggacaaaacgggactggctagttaaacaaagaggctgggatgggtttgtggagttcttccatgtagaggacctagaaggtggcatcaggaatgtgctgctggcttttgcaggtgttgctggagtaggagctggtttggcatatctaataagatag

SEQ ID NO: 13 - protein, 205 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWVGALGDVSLG

SEQ ID NO: 14 - DNA, 618 bp, Homo sapiens
atggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggtaggtgcacttggtgatgtgagtctgggctga

SEQ ID NO: 15 - protein, 138 aa, Homo sapiens
MAKQPSDVSSECDREGRQLQPAERPPQLRPGAPTSLQTEPQDRSPAPMSCDKSTQTPSPPCQAFNHYLSAMASMRQAEPADMRPEIWIAQELRRIGDEFNAYYARRVFLNNYQAAEDHPRMVILRLLRYIVRLVWRMH

SEQ ID NO: 16 - DNA, 417 bp, Homo sapiens
atggcaaagcaaccttctgatgtaagttctgagtgtgaccgagaaggtagacaattgcagcctgcggagaggcctccccagctcagacctggggcccctacctccctacagacagagccacaagacaggagcccagcacccatgagttgtgacaaatcaacacaaaccccaagtcctccttgccaggccttcaaccactatctcagtgcaatggcttccatgaggcaggctgaacctgcagatatgcgcccagagatatggatcgcccaagagttgcggcgtattggagacgagtttaacgcttactatgcaaggagggtatttttgaataattaccaagcagccgaagaccacccacgaatggttatcttacgactgttacgttacattgtccgcctggtgtggagaatgcattga

SEQ ID NO: 17 - protein, 350 aa, Homo sapiens
MFGLKRNAVIGLNLYCGGAGLGAGSGGATRPGGRLLATEKEASARREIGGGEAGAVIGGSAGASPPSTLTPDSRRVARPPPIGAEVPDVTATPARLLFFAPTRRAAPLEEMEAPAADAIMSPEEELDGYEPEPLGKRPAVLPLLELVGESGNNTSTDGSLPSTPPPAEEEEDELYRQSLEIISRYLREQATGAKDTKPMGRSGATSRKALETLRRVGDGVQRNHETAFQGMLRKLDIKNEDDVKSLSRVMIHVFSDGVTNWGRIVTLISFGAFVAKHLKTINQESCIEPLAESITDVLVRTKRDWLVKQRGWDGFVEFFHVEDLEGGIRNVLLAFAGVAGVGAGLAYLIR

SEQ ID NO: 18 - DNA, 1053 bp, Homo sapiens
atgtttggcctcaaaagaaacgcggtaatcggactcaacctctactgtgggggggccggcttgggggccggcagcggcggcgccacccgcccgggagggcgacttttggctacggagaaggaggcctcggcccggcgagagatagggggaggggaggccggcgcggtgattggcggaagcgccggcgcaagccccccgtccaccctcacgccagactcccggagggtcgcgcggccgccgcccattggcgccgaggtccccgacgtcaccgcgacccccgcgaggctgcttttcttcgcgcccacccgccgcgcggcgccgcttgaggagatggaagccccggccgctgacgccatcatgtcgcccgaagaggagctggacgggtacgagccggagcctctcgggaagcggccggctgtcctgccgctgctggagttggtcggggaatctggtaataacaccagtacggacgggtcactaccctcgacgccgccgccagcagaggaggaggaggacgagttgtaccggcagtcgctggagattatctctcggtaccttcgggagcaggccaccggcgccaaggacacaaagccaatgggcaggtctggggccaccagcaggaaggcgctggagaccttacgacgggttggggatggcgtgcagcgcaaccacgagacggccttccaaggcatgcttcggaaactggacatcaaaaacgaagacgatgtgaaatcgttgtctcgagtgatgatccatgttttcagcgacggcgtaacaaactggggcaggattgtgactctcatttcttttggtgcctttgtggctaaacacttgaagaccataaaccaagaaagctgcatcgaaccattagcagaaagtatcacagacgttctcgtaaggacaaaacgggactggctagttaaacaaagaggctgggatgggtttgtggagttcttccatgtagaggacctagaaggtggcatcaggaatgtgctgctggcttttgcaggtgttgctggagtaggagctggtttggcatatctaataagatag

SEQ ID NO: 19 - protein, 271 aa, Homo sapiens
MFGLKRNAVIGLNLYCGGAGLGAGSGGATRPGGRLLATEKEASARREIGGGEAGAVIGGSAGASPPSTLTPDSRRVARPPPIGAEVPDVTATPARLLFFAPTRRAAPLEEMEAPAADAIMSPEEELDGYEPEPLGKRPAVLPLLELVGESGNNTSTDGSLPSTPPPAEEEEDELYRQSLEIISRYLREQATGAKDTKPMGRSGATSRKALETLRRVGDGVQRNHETAFQGWVCGVLPCRGPRRWHQECAAGFCRCCWSRSWFGISNKIALL

SEQ ID NO: 20 - DNA, 816 bp, Homo sapiens
atgtttggcctcaaaagaaacgcggtaatcggactcaacctctactgtgggggggccggcttgggggccggcagcggcggcgccacccgcccgggagggcgacttttggctacggagaaggaggcctcggcccggcgagagatagggggaggggaggccggcgcggtgattggcggaagcgccggcgcaagccccccgtccaccctcacgccagactcccggagggtcgcgcggccgccgcccattggcgccgaggtccccgacgtcaccgcgacccccgcgaggctgcttttcttcgcgcccacccgccgcgcggcgccgcttgaggagatggaagccccggccgctgacgccatcatgtcgcccgaagaggagctggacgggtacgagccggagcctctcgggaagcggccggctgtcctgccgctgctggagttggtcggggaatctggtaataacaccagtacggacgggtcactaccctcgacgccgccgccagcagaggaggaggaggacgagttgtaccggcagtcgctggagattatctctcggtaccttcgggagcaggccaccggcgccaaggacacaaagccaatgggcaggtctggggccaccagcaggaaggcgctggagaccttacgacgggttggggatggcgtgcagcgcaaccacgagacggccttccaaggatgggtttgtggagttcttccatgtagaggacctagaaggtggcatcaggaatgtgctgctggcttttgcaggtgttgctggagtaggagctggtttggcatatctaataagatagccttactgtaa

(19) United States
(12) Patent Application Publication
(10) Pub. No.: US 2022/0233558 A1
(43) Pub. Date: Jul. 28, 2022

(54) METHOD AND COMPOSITION FOR PREDICTING EFFICACY OF BCL2/BCL-XL INHIBITORS ON CANCER

(71) Applicants: Ascentage Pharma (Suzhou) Co., Ltd., Suzhou (CN); Ascentage Pharma Group Corp Limited, Hong Kong (CN)

(72) Inventors: Yifan ZHAI, Suzhou (CN); Dajun YANG, Suzhou (CN); Jing DENG, Suzhou (CN); Douglas Dong FANG, Suzhou (CN)

(21) Appl. No.: 17/610,552

(22) PCT Filed: May 12, 2020
(86) PCT No.: PCT/CN2020/089771

§ 371 Date: Nov. 11, 2021

(30) Foreign Application Priority Data
May 13, 2019 (CN) PCT/CN2019/086673
Apr. 28, 2020 (CN) 202010350281.9

(51) Int. Cl. A61K 31/675; A61K 31/496; A61K 45/06; G01N 33/574

(52) U.S. Cl. CPC A61K 31/675; A61K 31/496; G01N 2800/7028; G01N 33/57426; A61K 45/06

(57) ABSTRACT

Provided are biomarkers for predicting the efficacy of BCL-2/BCL-XL dual or selective inhibitors in treating cancer patients. The biomarkers comprise a complex comprising BCL-2 or BCL-XL. Also provided are methods and compositions, e.g., kits, for evaluating levels of the biomarkers and methods of using such levels to predict a cancer patient's response to the BCL-2/BCL-XL dual inhibitors or BCL-XL or BCL-2 inhibitors. Such information can be used in determining prognosis and treatment options for cancer patients.

### FIELD OF THE INVENTION

The present invention generally relates to biomarkers for cancer treatment.

### BACKGROUND

The evasion of apoptosis is a hallmark of human cancer and is a frequent cause of therapeutic resistance (Hanahan D et al, Cell (2000) 100:57-70; Delbridge A R et al, Cold Spring Harb Perspect Biol (2012) 4). Therefore, targeting key apoptosis promoters in human cancer is an attractive strategy for the development of entirely new classes of anticancer therapies.

BCL-2 (B cell lymphoma protein 2) family proteins are the key regulators of apoptosis in the mitochondria-mediated pathway. The BCL-2 family proteins include the anti-apoptotic (pro-survival) members, including BCL-2, BCL-XL, BCL-w, MCL-1 and A1, and the pro-apoptotic (pro-death) members. The balance between anti-apoptotic (pro-survival) and pro-apoptotic (pro-death) proteins dictates the fate of the cell to live or die. Overexpression of pro-survival proteins, such as BCL-2 and BCL-XL, has been correlated with tumorigenesis and is a frequent cause of resistance to anticancer therapies (Vaux D L et al, Nature (1988) 335:440-42; Delbridge A R et al, Cell Death Differ (2015) 22:1071-80). Thus, agents designed to target the anti-apoptotic BCL-2 proteins, such as small-molecule BH3 mimetics, may provide new strategies for the treatment of cancer patients. Clinical available BCL-2 inhibitors or BH3 mimetics including those undergoing clinical evaluation however showed limited efficacy in hematological cancer as well as solid tumor, possibly due to the complicated signaling pathways and tumor microenvironment.

Clinical responses to anticancer therapies are often restricted to a subset of patients. To maximize the efficiency of anticancer therapy, personalized chemotherapy based on molecular biomarkers has been proposed. However, the identification of predicative biomarkers capable of predicting response to cancer chemotherapy still remains a challenge. Therefore, there is a continuing need for development of biomarkers for predicting efficacy of BCL-XL/BCL-2 dual inhibitors, BCL-XL inhibitors, or BCL-2 inhibitors on cancer treatment.

### SUMMARY OF INVENTION

The present disclosure in one aspect provides a method for treating cancer in a subject in need thereof. In one embodiment, the method comprises: (a) measuring a test level of at least one biomarker comprising a first complex comprising BCL-XL or BCL-2 protein, in a test sample comprising a cell obtained from the subject; (b) comparing the test level of the at least one biomarker with a corresponding reference level of the at least one biomarker to determine a difference; and (c) administering a BCL-2/BCL-XL dual inhibitor or a BCL-XL inhibitor or a BCL-2 inhibitor to the subject when the difference reaches a threshold. In another embodiment, the method comprises: (a) measuring a baseline level of at least one biomarker comprising a first complex comprising BCL-XL or BCL-2 protein, in a test sample comprising a cell obtained from the subject; (b) treating the test sample with a BCL-2/BCL-XL dual inhibitor or a BCL-XL inhibitor or a BCL-2 inhibitor, (c) measuring a post-treatment level of at least one biomarker in the treated test sample; (d) comparing the post-treatment level with the baseline level of the at least one biomarker to determine post-treatment change in the level of the at least one biomarker; and (e) administering a BCL-2/BCL-XL dual inhibitor or a BCL-XL inhibitor or a BCL-2 inhibitor to the subject when the post-treatment change reaches a threshold.

In another aspect, the present disclosure provides a method for identifying and/or selecting a subject having cancer for treatment with a BCL-2/BCL-XL dual inhibitor or a BCL-XL or BCL-2 inhibitor. In one embodiment, the method comprises: (a) measuring a test level of at least one biomarker comprising a first complex comprising BCL-XL or BCL-2 protein, in a test sample comprising a cell obtained from the subject; (b) comparing the test level of the at least one biomarker with a corresponding reference level of the at least one biomarker to determine a difference; and (c) determining that the subject is likely to respond to the treatment with the BCL-2/BCL-XL dual inhibitor or the BCL-XL inhibitor or the BCL-2 inhibitor when the difference reaches a threshold. In another embodiment, the method comprises: (a) measuring a baseline level of at least one biomarker comprising a first complex comprising BCL-XL or BCL-2 protein, in a test sample comprising a cell obtained from the subject; (b) treating the test sample with a BCL-2/BCL-XL dual inhibitor or a BCL-XL inhibitor or a BCL-2 inhibitor, (c) measuring a post-treatment level of at least one biomarker in the treated test sample; (d) comparing the post-treatment level with the baseline level of the at least one biomarker to determine post-treatment change in the level of the at least one biomarker; and (e) determining that the subject is likely to respond to the treatment with the BCL-2/BCL-XL dual inhibitor or the BCL-XL inhibitor or the BCL-2 inhibitor when the post-treatment change reaches a threshold.

In yet another aspect, the present disclosure provides a method for monitoring therapeutic efficacy in a subject having cancer and having been treated with a BCL-2/BCL-XL dual inhibitor or a BCL-XL or BCL-2 inhibitor for a therapeutic period. In one embodiment, the method comprises: (a) obtaining a test sample comprising a cell from the subject after the therapeutic period; (b) measuring a level of at least one biomarker comprising a first complex comprising BCL-X1 or BCL-2 in the test sample to obtain a post-treatment level of the at least one biomarker; (c) comparing the post-treatment level with a baseline level of the at least one biomarker measured on a test sample obtained from the subject before the therapeutic period, to determine post-treatment change in the level of the at least one biomarker; and (d) continuing administering the BCL-2/BCL-XL dual inhibitor or the BCL-XL or the BCL-2 inhibitor to the subject when the post-treatment change reaches a threshold, or when the post-treatment change does not reach the threshold, increasing the dose or the dosing frequency of the BCL-2/BCL-XL dual inhibitor or the BCL-XL inhibitor or the BCL-2 inhibitor to the subject, administering a second anti-cancer therapeutic agent in combination to the BCL-2/BCL-XL dual inhibitor or the BCL-XL inhibitor or the BCL-2 inhibitor to the subject, or discontinuing administering the BCL-2/BCL-XL dual inhibitor or the BCL-XL inhibitor or the BCL-2 inhibitor to the subject.

In certain embodiments, the at least one biomarker further comprises a second complex comprising BCL-XL or BCL-2 protein.

In certain embodiments, wherein the first and/or the second complex comprises BCL-XL protein complexed with a BH3-only protein, BCL-2 protein complexed with a BH3-only protein, BCL-XL protein complexed with a BH3-containing protein, or BCL-2 protein complexed with a BH3-containing protein.

In certain embodiments, the BH3-only protein is selected from the group consisting of: BIM, BID, BAD, BIK, HRK, BMF, and PUMA. In certain embodiments, the BH3 domain containing protein comprises BAX or BAK.

In certain embodiments, the at least one biomarker comprises two or more complexes selected from the group consisting of: BCL-XL:BIM, BCL-XL:PUMA, BCL-2:BIM, BCL-2:PUMA, MCL-1:BIM, MCL-1:PUMA, and any combination thereof.

In certain embodiments, the level of the at least one biomarker comprises combination of the level of the first complex and the level of the second complex.

In certain embodiments, the level of the complex is measured by using protein-protein interaction assay. In certain embodiments, the protein-protein interaction assay is based on immunoassay or proximity assays. In certain embodiments, the protein-protein interaction assay is meso scale discovery (MSD) advanced enzyme-linked immunosorbent assay (MSD-ELISA), standard complex ELSIA, proximity ligation assay, co-immunoprecipitation, immunoblotting assay, or cross-linking assay. In certain embodiments, the level of the first and/or the second complex is measured by using an antibody that specifically bind to the complex or to the BCL-XL protein or to the BCL-2 protein.

In certain embodiments, the first and/or the second complex is a dominant complex in the sample. In certain embodiments, the cancer is blood cancer and the dominant complex comprises a complex of BCL-2:BIM. In certain embodiments, the cancer is solid tumor and the dominant complex comprises a complex of BCl-xL:BIM, and/or a complex of BCl-xL:PUMA.

In certain embodiments, the at least one biomarker further comprises MCL-1.

In certain embodiments, the at least one biomarker further comprises BCL-2 or BCL-XL. In certain embodiments, the level of MCL-1, BCL-2 or BCL-XL is measured at mRNA level, protein level or DNA level.

In certain embodiments, the level of MCL-1, BCL-2 or BCL-XL is measured by an amplification assay, a hybridization assay, a sequencing assay, an immunoassay, a spectrometry method, or a proximity assay.

In certain embodiments, the cancer is a solid tumor. In certain embodiments, the solid tumor is lung cancer, gastric cancer, esophageal cancer, colon cancer, cholangiocarcinoma, liver cancer, breast cancer, cervical cancer, ovarian cancer, head and neck cancer or brain tumors.

In certain embodiments, the cancer is a blood cancer. In certain embodiments, the blood cancer is chronic lymphocytic leukemia (CLL), acute myeloid leukemia (AML), multiple myeloma (MM), Waldenstrom macroglobulinemia (WM), acute lymphoblastic leukemia (ALL) or lymphoma.

In certain embodiments, the reference level is an average level of the at least one biomarker in representative samples of the same type of cancer. In certain embodiments, the reference level of the at least one biomarker is an empirical level of the biomarker in a tumor sample of the same type or in a certain type of cancer (e.g. in blood cancer) or in general cancer.

In certain embodiments, the test sample is a bodily fluid sample or a tissue sample.

In certain embodiments, the BCL-2/BCL-XL dual inhibitor is a compound having a structure of formula (I), (II), (III), or (IV), as defined herein. In certain embodiments, the BCL-2/BCL-XL dual inhibitor is Compound A or Compound B, as provided herein. In certain embodiments, the BCL-2 inhibitor is a compound having a structure of formula (V), as defined herein. In certain embodiments, the BCL-2 inhibitor is Compound C, as provided herein.

In certain embodiments, the BCL-2/BCL-xL dual inhibitor is (R)-2-(1-(3-(4-(N-(4-(4-(3-(2-(4-chlorophenyl)-1-isopropyl-5-methyl-4-(methylsulfonyl)-1H-pyrrol-3-yl)-5-fluorophenyl)piperazin-1-yl)phenyl)sulfamoyl)-2-(trifluoromethylsulfonyl)phenylamino)-4-(phenylthiol)butyl)piperidine-4-carbonyloxy)ethylphosphonic acid or a pharmaceutically acceptable salt thereof (also referred to as “Compound A” herein).

In certain embodiments, the BCL-2/BCL-xL dual inhibitor is (R)-1-(3-(4-(N-(4-(4-(3-(2-(4-chlorophenyl)-1-isopropyl-5-methyl-4-(methylsulfonyl)-1H-pyrrol-3-yl)-5-fluorophenyl) piperazin-1-yl)phenyl) sulfamoyl)-2-(trifluoromethylsulfonyl)phenylamino)-4-(phenylthio)butyl)piperldine-4-carboxylic acid or a pharmaceutically acceptable salt thereof (also referred to as “Compound B” herein).

In certain embodiments, the BCL-xL inhibitor is (S)-N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide or a pharmaceutically acceptable salt thereof (also referred to as “Compound C” herein).

In yet another aspect, the present disclosure provides a kit for use in the methods described herein. In one embodiment, the kit comprises a first reagent for measuring a level of the complex. In certain embodiments, the first reagent comprises a first antibody that specifically binds to the complex or to the BCL-XL protein, or to the BCL-2 protein. In certain embodiments, at least one reagent further comprises a second reagent comprising a second antibody that specifically binds to the BH3-only protein or the BH3-domain containing protein in the complex.

In certain embodiments, the first antibody and/or the second antibody is detectably labeled. In certain embodiments, one of the first antibody and/or the second antibody is detectably labeled, and the other is capable of being captured. In certain embodiments, the at least one reagent further comprises a third reagent comprising a first oligonucleotide capable of hybridizing to the polynucleotide of MCL-1, or a third antibody capable of specifically binding to the protein of MCL-1.

In certain embodiments, the at least one reagent further comprises a fourth reagent comprising a second oligonucleotide capable of hybridizing to the polynucleotide of BCL-XL or BCL-2, or a fourth antibody capable of specifically binding to the protein of BCL-XL or BCL-2.

In another aspect, the present disclosure provides use of a reagent for measuring a level of at least one biomarker comprising a complex comprising BCL-XL protein or BCL-2 protein in the manufacture of a diagnostic kit for performing the method described herein.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A to 1C illustrate that summary of PDX trials on 11 solid tumor models (FIG. 1A), procedure of PDX trials for Pharmacodynamics (PD) and biomarker studies (FIG. 1B), and baseline levels of complexes (i.e. BCL-2:BIM, BCL-xL:BIM, BCL-2:PUMA, BCL-xL:PUMA, MCL-1:BIM, MCL-1:PUMA,) in solid tumor PDX samples and hematological cancer cell lines (Toledo) by MSD assay (FIG. 1C). “BCL-XLI^{amp}” means the sample has BCL-XL gene amplification in compare with the average levels, “MCL-XL^{nor}” means the sample has normal level of MCL-1 gene.

FIGS. 2A and 2B illustrate the change in the level of BCL-xL:BIM complex in Vehicle group and Compound A group by MSD assay.

FIGS. 3A to 3C illustrate the correlation of the Tumor Growth Inhibition (TGI) to Compound A and the baseline level of the protein complexes of: BCL-XL:BIM and BCL-XL:PUMA (FIG. 3A), or BCL-2:BIM and BCL-2:PUMA (FIG. 3B), or the combination of these baseline four complexes BCL-XL:BIM, BCL-XL:PUMA, BCL-2:BIM and BCL-2:PUMA (FIG. 3C).

FIG. 3D illustrates the correlation of the TGI and the combined level of BCL-XL:BIM, BCL-XL:PUMA, BCL-2:BIM and BCL-2:PUMA, the correlation of the TGI and MCL-1 protein baseline level and after-treatment change.

FIGS. 4A and 4B illustrates apoptosis protein expression levels (images in 4A) and the quantification (4B) in PDX samples by Western Blotting assay.

FIGS. 4C and 4D illustrates relative protein level of apoptosis (pro-death and anti-death) protein expression in Vehicle and Compound A groups by WB assay, and the quantification shown in FIG. 4E.

FIG. 5 illustrates the correlation of TGI and post-treatment change of BCL-xL:BIM and BCL-xL:PUMA, the two major complexes.

FIGS. 6A and 6B illustrate the results of MSD advanced ELISA analysis of BCL-2:BIM and BCL-XL:BIM complexes in Toledo (6A) and RS4;11 (6B) cells, which were treated with Compound B or ABT-737.

FIGS. 7A and 7B illustrate the results of MSD advanced ELISA analysis of BCL-2:BIM and BCL-XL:BIM complexes in Toledo (7A) and RS4;11 (7B) cells, which were treated with Compound B or ABT-263.

FIG. 8 shows exemplary sequences of the biomarkers as provided herein.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definitions

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, the singular forms “a”, “an” and “the” include plural references unless the context clearly dictates otherwise.

The term “biomarker” as used here refers to a biological molecule that is a measurable indicator of some biological state or condition. The term “biomarker” used herein is intended to encompass a polynucleotide of interest, a polypeptide (for example encoded by the polynucleotide of interest), and a complex containing two or more biomarkers in association together. Examples of biomarker provided herein can be a gene (e.g. genomic DNA, cDNA) or a product of the gene such as an mRNA transcribed from the gene, a protein encoded by the gene, and a protein complex. One specific example of the biomarkers provided herein include a complex comprising BCL-XL protein or BCL-2 protein. Another specific example of biomarkers provided herein include MCL-1, BCL-XL or BCL-2.

The term “complex” as used herein with respect to protein or polypeptide refers to a group of two or more associated polypeptide chains. Different polypeptide chains may have different functions. Typically, polypeptide chains in a protein complex are linked by non-covalent interactions. Different protein complexes may have different degrees of stability over time.

The term “BCL-XL” as used herein refers to BCL-XL gene and BCL-XL gene products such as mRNA of BCL-XL gene and protein encoded by BCL-XL gene. BCL-XL gene, also known as BCL-2 like 1 (BCL2L1) gene, encodes protein belonging to the BCL-2 protein family. The BCL-XL protein acts as an anti-apoptotic protein by preventing the release of mitochondrial contents such as cytochrome c. Human BCL-XL gene has a Gene ID of 598 in NCBI database. The mRNA transcripts of the human BCL-XL gene have NCBI reference sequences of XM_0111528964.2 and XM_017027993.1. The proteins encoded by the human BCL-XL gene have NCBI reference sequences of XP_011527266.1 and XP_016883482.1.

The term “BCL-2” as used herein refers to BCL-2 gene and BCL-2 gene products such as mRNA of BCL-2 gene and protein encoded by BCL-2 gene. BCL-2 gene encodes an integral outer mitochondrial membrane protein that blocks the apoptotic death of some cells such as lymphocytes. Human BCL-2 gene has a Gene ID of 596 in NCBI database. The mRNA transcripts of the human BCL-2 gene have NCBI reference sequences of XM_017025917.2 and XM_011526135.3. The proteins encoded by the human BCL-2 gene have NCBI reference sequences of XP_016881406.1 and XP_011524437.1.

The term “MCL-1” as used herein refers to MCL-1 gene and MCL-1 gene products such as mRNA of MCL-1 gene and protein encoded by MCL-1 gene. MCL-1 gene encodes proteins belonging to the BCL-2 family. Alternative splicing of the MCL-1 gene generates at least two proteins, the longer one enhancing cell survival by inhibiting apoptosis while the shorter protein promoting apoptosis and cell death. Human MCL-1 gene has a Gene ID of 4170 in NCBI database. The mRNA transcripts of the human MCL-1 gene have NCBI reference sequences of NM_021960.5, NM_182763.2 and NM_001197320.1. The proteins encoded by the human MCL-1 gene have NCBI reference sequences of NP_068779.1, NP_877495.1 and NP_001184249.1.

The term “BIM” as used herein refers to BIM gene and BIM gene products such as mRNA of BIM gene and protein encoded by BIM gene. BIM, also called BCL-2-like protein 11, is a pro-apoptotic BCL-2 family member that has been shown to interact with BCL-2, BCL-XL and MCL-1. Human BIM gene has a Gene ID of 10018 in NCBI database. The mRNA transcripts of the human BIM gene have NCBI reference sequences of NM_001204106, NM_001204107, NM_001204108, NM_001204109 and NM_001204110. The proteins encoded by the human BIM gene have NCBI reference sequences of NP_001191035, NP_001191036, NP_001191037, NP_001191038 and NP_001191039.

The term “PUMA” as used herein refers to PUMA gene and PUMA gene products such as mRNA of PUMA gene and protein encoded by PUMA gene. PUMA, or p53 Upregulated Modulator of Apoptosis, also known as Bcl-2-binding component 3 (BBC3), is a pro-apoptotic member of the BCL-2 protein family. The expression of PUMA is regulated by the tumor suppressor p53. After activation, PUMA interacts with anti-apoptotic BCL-2 family members, thus freeing pro-apoptotic molecules BAX and/or BAK which are then able to signal apoptosis to the mitochondria. Human PUMA gene has a Gene ID of 27113 in NCBI database. The mRNA transcripts of the human PUMA gene have NCBI reference sequences of NM_001127240, NM_001127241, NM_001127242 and NM_014417. The proteins encoded by the human PUMA gene have NCBI reference sequences of NP_001120712, NP_001120713, NP_001120714, NP_055232 and NP_001120712.1.

The term “BID” as used herein refers to BID gene and BID gene products such as mRNA of BID gene and protein encoded by BID gene. BID, or BH3 interacting-domain death agonist, is a pro-apoptotic member of the BCL-2 family that contains only the BH3 domain. In response to apoptotic signaling, BID interacts with another BCL-2 family protein, BAX, leading to the insertion of activated BAX into outer mitochondrial membrane. The anti-apoptotic BCL-2 family member, including BCL-2 itself, can bind BID and inhibit BID's ability to activate BAX. The expression of BID is upregulated by p53 and involved in p53-mediated apoptosis. Human BID gene has a Gene ID of 637 in NCBI database. The mRNA transcripts of the human BID gene have NCBI reference sequences of NM_001196, NM_001244567, NM_001244569, NM_001244570 and NM_001244572. The proteins encoded by the human BID gene have NCBI reference sequences of NP_001187, NP_001231496, NP_001231498, NP_001231499 and NP_001231501.

The term “BAD” as used herein refers to BAD gene and BIAD gene products such as mRNA of BAD gene and protein encoded by BAD gene. BAD, or BCL-2-associated death promoter, is a pro-apoptotic member of the BCL-2 family which is involved in initiating apoptosis. BAD is a member of the BH3-only family. Dephosphorylated BAD forms a heterodimer with BCL-2 and BCL-XL, inactivating them and thus allowing BAX/BAK triggered apoptosis. When BAD is phosphorylated by AKT, it forms the BAD-14-3-3 protein heterodimer, leaving BCL-2 free to inhibit BAX-triggered apoptosis. Human BAD gene has a Gene ID of 572 in NCBI database. The mRNA transcripts of the human BAD gene have NCBI reference sequences of NM_032989 and NM_004322. The proteins encoded by the human BAD gene have NCBI reference sequences of NP_004313 and NP_116784.

The term “BIK” as used herein refers to BIK gene and BIK gene products such as mRNA of BIK gene and protein encoded by BIK gene. BIK, or BCL-2-interacting killer is a pro-apoptotic member of the BCL-2 family. BIK interact with BCL-2 and BCL-XL. Human BIK gene has a Gene ID of 638 in NCBI database. The mRNA transcrips of the human BIK gene has NCBI reference sequence of NM_001197. The proteins encoded by the human BIK gene have NCBI reference sequences of NP_001188 and NP_001188.1.

The term “HRK” as used herein refers to HRK gene and HKR gene products such as mRNA of HRK gene and protein encoded by HRK gene. HRK, or HARAKIRI, is a pro-apoptotic protein that interacts with BCL-2 and BCL-XL. HRK protein lacks significant homology to other BCL-2 family members except for an 8-amino acid region that similar to BH3 domain of BIK. Human HRK gene has a Gene ID of 8739 in NCBI database. The mRNA transcript of the human HRK gene has an NCBI reference sequence of NM_003806. The protein encoded by the human HRK gene has an NCBI reference sequence of NP_003797.

The term “BMF” as used herein refers to BMF gene and BMF gene products such as mRNA of BMF gene and protein encoded by BMF gene. BMF, or BCL-2-modified factor is a BCL-2 family member that contains a single BH3 domain. BMF has been shown to bind BCL-2 protein and act as an apoptotic activator. Human BMF gene has a Gene ID of 90427 in NCBI database. The mRNA transcripts of the human BMF gene have NCBI reference sequences of NM_001003940, NM_001003942, NM_001003943 and NM_033503. The proteins encoded by the human BMF gene have NCBI reference sequences of NP_001003940, NP_001003942, NP_001003943 and NP_277038.

The term “BAK” as used herein refers to BAK gene and BAK gene products such as mRNA of BAK gene and protein encoded by BAK gene. BAK, also called BCL-2 homologous antagonist/killer, is a pro-apoptotic member of BCL-2 family. BAK protein interacts with and accelerates the opening of the mitochondrial voltage-dependent anion channel, which leads to a loss in membrane potential and the release of cytochrome c. Human BAK gene has a Gene ID of 578 in NCBI database. The mRNA transcript of the human BAK gene has NCBI reference sequence of NM_001188. The protein encoded by the human BAK gene has NCBI reference sequence of NP_001179.

The term “BAX” as used herein refers to BAX gene and BAX gene products such as mRNA of BAX gene and protein encoded by BAX gene. BAX, also known as BCL-like protein 4, is a pro-apoptotic member of BCL-2 family. BAX protein forms a heterodimer with BCL-2 and increases the opening of the mitochondrial voltage-dependent anion channel, which leads to the loss in membrane potential and the release of cytochrome c. The expression of BAX gene is regulated by p53 and has been shown to be involved in p53-mediated apoptosis. Human BAX gene has a Gene ID of 581 in NCBI database. The mRNA transcripts of the human BAX gene have NCBI reference sequences of NM_001291428, NM_001291429, NM_001291430, NM_001291431 and NM_004324. The proteins encoded by the human BAX gene have NCBI reference sequences of NP_001278357, NP_001278358, NP_001278359, NP_001278360 and NP_004315.

The term “level” with respect to a biomarker, refers to the amount or quantity of the biomarker of interest present in a sample. Such amount or quantity may be expressed in the absolute terms, i.e., the total quantity of the biomarker in the sample, or in the relative terms, i.e., the concentration or percentage of the biomarker in the sample. Level of a biomarker can be measured at DNA level (for example, as represented by the amount or quantity or copy number of the gene in a chromosomal region), at RNA level (for example as mRNA amount or quantity), or at protein level (for example as protein or protein complex amount or quantity).

As used herein, the term “cancer” refers to any diseases involving an abnormal cell growth and include all stages and all forms of the disease that affects any tissue, organ or cell in the body. The term includes all known cancers and neoplastic conditions, whether characterized as malignant, benign, soft tissue, or solid, and cancers of all stages and grades including pre- and post-metastatic cancers. In general, cancers can be categorized according to the tissue or organ from which the cancer is located or originated and morphology of cancerous tissues and cells.

The term “solid tumor” as used herein refers to any cancer that does not contain cysts or liquid areas. Solid tumor generally does not include leukemias (i.e. blood cancer). Solid tumor can be benign or malignant. As used herein, types of solid tumor include, without limitation, adrenocortical carcinoma, anal cancer, astrocytoma, childhood cerebellar or cerebral, basal-cell carcinoma, bile duct cancer, bladder cancer, bone tumor, brain cancer, cerebellar astrocytoma, cerebral astrocytoma/malignant glioma, ependymoma, medulloblastoma, supratentorial primitive neuroectodermal tumors, visual pathway and hypothalamic glioma, breast cancer, Burkitt's lymphoma, ervical cancer, colon cancer, emphysema, endometrial cancer, ependymoma, esophageal cancer, Ewing's sarcoma, retinoblastoma, gastric (stomach) cancer, glioma, head and neck cancer, heart cancer, Hodgkin lymphoma, islet cell carcinoma (endocrine pancreas), Kaposi sarcoma, kidney cancer (renal cell cancer), laryngeal cancer, liver cancer, lung cancer, neuroblastoma, non-Hodgkin lymphoma, ovarian cancer, pancreatic cancer, pharyngeal cancer, prostate cancer, rectal cancer, renal cell carcinoma (kidney cancer), retinoblastoma, Ewing family of tumors, skin cancer, stomach cancer, testicular cancer, throat cancer, thyroid cancer, and vaginal cancer.

It is noted that in this disclosure, terms such as “comprises”, “comprised”, “comprising”, “contains”, “containing” and the like are intended to be inclusive or open-ended, and do not exclude additional, un-recited elements or method steps.

The terms “determining,” “assessing,” “measuring” and “detecting” can be used interchangeably and refer to both quantitative and semi-quantitative determinations. Where either a quantitative and semi-quantitative determination is intended, the phrase “determining a level” of a polynucleotide or polypeptide of interest or “detecting” a polynucleotide or polypeptide of interest can be used.

The term “hybridizing” refers to the binding, duplexing, or hybridizing of a nucleic acid molecule preferentially to a particular nucleotide sequence under stringent conditions. The term “stringent conditions” refers to hybridization and wash conditions under which a probe will hybridize preferentially to its target subsequence, and to a lesser extent to, or not at all to, other sequences in a mixed population (e.g., a cell lysate or DNA preparation from a tissue biopsy). A stringent condition in the context of nucleic acid hybridization are sequence dependent, and are different under different environmental parameters. An extensive guide to the hybridization of nucleic acids is found in, e.g., Tijssen Laboratory Techniques in Biochemistry and Molecular Biology—Hybridization with Nucleic Acid Probes part I, Ch. 2, “Overview of principles of hybridization and the strategy of nucleic acidprobe assays,” (1993) Elsevier, N.Y. Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe.

The term “nucleic acid” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, shRNA, single-stranded short or long RNAs, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

The term “complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%>, 70%>, 80%>, 90%, and 100% complementary).

The term “prognose” or “prognosing” as used herein refers to the prediction or forecast of the future course or outcome of a disease or condition.

In general, a “protein” is a polypeptide (i.e., a string of at least two amino acids linked to one another by peptide bonds). Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means.

The term “responsive” or “responsiveness” as used in the context of a patient's response to a cancer therapy, are used interchangeably and refer to a beneficial patient response to a treatment as opposed to unfavorable responses, i.e. adverse events. In a patient, beneficial response can be expressed in terms of a number of clinical parameters, including loss of detectable tumor (complete response), decrease in tumor size and/or cancer cell number (partial response), tumor growth arrest (stable disease), enhancement of anti-tumor immune response, possibly resulting in regression or rejection of the tumor; relief, to some extent, of one or more symptoms associated with the tumor; increase in the length of survival following treatment; and/or decreased mortality at a given point of time following treatment. Continued increase in tumor size and/or cancer cell number and/or tumor metastasis is indicative of lack of beneficial response to treatment, and therefore decreased responsiveness.

The term “sample” as used herein refers to a biological sample that is obtained from a subject and contains one or more biomarker(s) of interest. Examples of sample include, without limitation, bodily fluid, such as blood, plasma, serum, urine, vaginal fluid, uterine or vaginal flushing fluids, plural fluid, ascitic fluid, cerebrospinal fluid, saliva, sweat, tears, sputum, bronchioalveolar lavage fluid, etc., and tissues, such as biopsy tissue (e.g. biopsied bone tissue, bone marrow, breast tissue, gastroinstetinal tract tissue, lung tissue, liver tissue, prostate tissue, brain tissue, nerve tissue, meningeal tissue, renal tissue, endometrial tissue, cervical dittuse, lymph node tissue, muscle tissue, or skin tissue), a paraffin embedded tissue. In certain embodiments, the sample can be a biological sample comprising cells (for example cancer cells or non-cancer cells such as peripheral blood mononuclear cells (PBMC)). In some embodiments, the sample is a fresh or archived sample obtained from a tumor, e.g., by a tumor biopsy or fine needle aspirate. The sample also can be any biological fluid containing cancer cells. The collection of a sample from a subject is performed in accordance with the standard protocol generally followed by hospital or clinics, such as during a biopsy.

The term “test sample” as used herein refers to a sample obtained from a subject in need of cancer treatment and is representative of the cancer condition of the subject. For example, the test sample can contain a cancer cell.

As used herein, the term “subject” refers to a human or any non-human animal (e.g., mouse, rat, rabbit, dog, cat, cattle, swine, sheep, horse or primate). In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “treatment,” “treat,” or “treating” as used herein refers to preventing or alleviating a condition, slowing the onset or rate of development of a condition, preventing or delaying the development of symptoms associated with a condition, reducing or ending symptoms associated with a condition, generating a complete or partial regression of a condition, curing a condition, or some combination thereof. With regard to cancer, “treating” or “treatment” may refer to inhibiting or slowing neoplastic or malignant cell growth, proliferation, or metastasis, preventing or delaying the development of neoplastic or malignant cell growth, proliferation, metastasis, or cancer symptoms or some combination thereof. Thus, in the disclosed method, treatment can refer to a 10%, 20%, 30%, 40%, 50%, 60%, 70%), 80%), 90%), or 100% reduction in the severity of a cancer or symptom of the cancer. For example, a method of treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100% or any percent reduction between 10 and 100% as compared to native or control levels. It is understood that treatment does not necessarily refer to a cure or complete ablation of the disease, condition, or symptoms of the disease or condition.

The term “detectable label” as used herein refers to a molecule or moiety that allows detection. The term “detectably labeled” with respect to a reagent means that the reagent comprises a detectable label or can be bound by a detectable label. Detectable labels can be useful in labelling proteins such as antibodies and nucleic acids such as probes and primers. Examples of the detectable label suitable for labeling primers, probes and antibodies include, for example, chromophores, radioisotopes, fluorophores, chemiluminescent moieties, particles (visible or fluorescent), nucleic acids, ligand, or catalysts such as enzymes.

Examples of radioisotopes include, without limitation, ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P.

Examples of fluorophores include, without limitation, Acridine, 7-amino-4-methylcoumarin-3-acetic acid (AMCA), BODIPY, Cascade Blue, Cy2, Cy3, Cy5, Cy7, Edans, Eosin, Erythrosin, Fluorescein, 6-FAM, TET, JOC, HEX, Oregon Green, Rhodamine, Rhodol Green, Tamra. Rox, and Texas Red™ (Molecular Probes, Inc., Eugene, Oreg.).

Examples of enzymes include, without limitation, alkaline phosphatase, acid phosphatase, horseradish peroxidase, beta-galactosidase, and ribonuclease.

Examples of ligands include, without limitation, biotin, avidin, nucleic acid, oligonucleotide, an antibody or an antigen.

It should be understood that it is not necessary for a detectable label to produce a detectable signal, for example, in some embodiments, it may can react with a detectable partner or react with one or more additional compounds to generate a detectable signal. For example, the detectable label can be a ligand capable of functioning as a specific binding partner for a labeled ligand (e.g. a secondary labeled antibody). For another example, enzymes are useful a detectable label due to their catalytic activity to catalyze chromo-, fluoro-, or lumo-genic substrate which results in generation of a detectable signal.

Biomarkers for Predicting Efficacy of BCL-2/BCL-XL Inhibitor

The methods and compositions described herein are based, in part, on the discovery of biomarkers whose level is predictive of anti-cancer efficacy of BCL-2 inhibitor, BCL-XL inhibitor, or BCL-2/BCL-XL dual inhibitors. In particular, the biomarkers described herein are useful for predicting anti-cancer efficacy of BCL-2/BCL-XL dual inhibitors, BCL-XL inhibitors and/or BCL-2 inhibitors, in particular those as provided herein (e.g. those as disclosed in WO 2014113413 A1, U.S. Pat. No. 9,403,856B, and WO 2018027097 A1, which are incorporated herein to the entirety).

Bcl-2 family of proteins are key regulators of the mitochondrial (also called “intrinsic”) pathway of apoptosis. Their activity is linked to the onset of lymphoid and several solid tumor cancers and is believed in many cancers to be the key mediator of resistance to chemotherapy. The BCL-2 family of proteins are characterized in the structural homology domains BH1, BH2, BH3 and BH4, and can be further classified into three subfamilies depending on how many of the homology domains each protein contains and on its biological activity, i.e., whether it has pro- or anti-apoptotic function.

The first subgroup of BCL-2 proteins contains proteins having all four homology domains, i.e., BH1, BH2, BH3 and BH4. Their general effect is anti-apoptotic, that is, to preserve a cell from starting a cell death process. Proteins such as BCL-2, BCL-W, BCL-XL, MCL-1, and BFL-1/A1 are members of this first subgroup.

Proteins belonging to the second subgroup of Bcl-2 proteins contain the three homology domains BH1, BH2, and BH3, and have a pro-apoptotic effect. The two main representative proteins of this second subgroup are BAX and BAK. This group is also called multi-domain or BH3-domain containing pro-death proteins in the present disclosure.

The third subgroup of Bcl-2 proteins is composed of proteins containing only the BH3 domain and members of this subgroup are usually referred to as “BH3-only proteins.” Their biological effect on the cell is pro-apoptotic. BIM, BID BAD, BIK, NOXA, HRK, BMF, and PUMA are examples of this third subfamily of proteins.

Dysregulation of the apoptotic pathway causes survival of the affected cells which would otherwise have undergone apoptosis in normal conditions. As there are too many BCL-2 family proteins involved in the apoptotic pathway, and the natural levels of these proteins can vary in different cell types, there is few biomarker that is generally applicable for predicting anti-cancer efficacy of a particular BCL-2 inhibitor, a BCL-XL inhibitor or a BCL-2/BCL-XL dual inhibitors.

The inventors of the present disclosure surprisingly found that the level of at least one biomarker comprising a complex comprising BCL-XL or BCL-2 is correlated to the anti-cancer efficacy of BCL-2/BCL-XL dual inhibitors, BCL-XL inhibitors and/or BCL-2 inhibitors, in particular, for the inhibitors provided herein.

In certain embodiments, the at least one biomarker provided herein comprises a first complex comprising BCL-XL or BCL-2 protein. In certain embodiments, the at least one biomarker provided herein further comprises a second complex comprising BCL-XL or BCL-2 protein. In certain embodiments, the first and/or the second complex can be complexed with a BH3-only protein or with a BH3-domain containing protein. In certain embodiments, the first and/or the second complex comprises BCL-XL protein complexed with a BH3-only protein, BCL-2 protein complexed with a BH3-only protein, BCL-XL protein complexed with a BH3-containing protein, or BCL-XL protein complexed with a BH3-containing protein.

BH3-only proteins are categorized as either “activator,” e.g., BIM and BID, or “sensitizer,” e.g., BAD, BIK, NOXA, HRK, BMF, and PUMA, depending on their regulatory function. Activator proteins can bind to and activate pro-apoptotic proteins, but these activator proteins can also bind to anti-apoptotic BCL-2 family proteins (such as BCL-2 or BCL-xL) and get sequestered and prevented from exerting their pro-apoptotic activity. Sensitizer proteins can displace the activator proteins from the anti-apoptotic BCL-2 family proteins (such as BCL-2 or BCL-xL) and thereby blocking the anti-apoptotic activity. In certain embodiments, the BH3-only protein is selected from the group consisting of: BIM, BID, BAD, BIK, HRK, BMF, and PUMA.

BH3-domain containing proteins contain the three homology domains BH1, BH2, and BH3, and have a pro-apoptotic effect. BH3-domain containing proteins can be selected from BAX and BAK. In certain embodiments, the BH3-domain containing protein is selected from the group consisting of: BAK, and BAX.

In certain embodiments, the at least one biomarker comprises two or more complexes selected from the group consisting of: BCL-XL:BIM, BCL-XL:PUMA, BCL-2:BIM, BCL-2:PUMA, MCL-1:BIM, MCL-1:PUMA, and any combination thereof.

In certain embodiments, BCL-2 comprise an amino acid sequence of SEQ ID NO: 1 or 13. In certain embodiments, BCL-XL comprise an amino acid sequence of SEQ ID NO: 3. In certain embodiments, BIM comprise an amino acid sequence of SEQ ID NO: 5 or 15. In certain embodiments, BAD comprise an amino acid sequence of SEQ ID NO: 7. In certain embodiments, PUMA comprise an amino acid sequence of SEQ ID NO: 9.

In certain embodiments, the at least one biomarker provided herein further comprises MCL-1.

In certain embodiments, MCL-1 comprises a gene sequence of SEQ ID NO: 12, 18 or 20, which encodes a protein having an amino acid sequence of SEQ ID NO: 11, 17 or 19. MCL-1 used as the biomarker herein can be a polynucleotide or a protein of MCL-1, or a complex (e.g. protein complex) comprising MCL-1.

In certain embodiments, the at least one biomarker provided herein further comprises BCL-2 or BCL-XL. BCL-2 or BCL-XL used as the biomarker herein can be a polynucleotide or a protein of BCL-2 or BCL-XL.

Therefore, the present disclosure provides at least one detection reagent for measuring the level of the at least one biomarker provided herein, and methods for identifying and/or selecting a subject having or suspected of having cancer for treatment with a BCL-2/BCL-XL dual inhibitor or a BCL-XL inhibitor or a BCL-2 inhibitor provided herein, methods for treating cancer in the subject in need thereof, and methods for monitoring therapeutic efficacy in a subject having cancer and having been treated with a BCL-2/BCL-XL dual inhibitor or a BCL-XL inhibitor or a BCL-2 inhibitor provided herein for a therapeutic period, based on the measured level of the at least one biomarker provided herein.

Reagents for Measuring Level of the Biomarkers

In one aspect, the present disclosure provides at least one reagent for detecting or measuring the level of the at least one biomarker provided herein.

In certain embodiments, the at least one reagent comprise a first reagent comprising a first antibody that specifically bind to the complex, or to the BCL-XL protein, or to the BCL-2 protein. In certain embodiments, the first antibody provided herein comprise an antigen-binding region capable of specifically binding to an epitope within the protein or polypeptide having a sequence selected from: SEQ ID NOs: 1, 3, 5, 7, 9, 11, 13, 15, 17, and 19.

In certain embodiments, the at least one reagent further comprises a second reagent comprising a second antibody that specifically binds to the BH3-only protein or the BH3-domain containing protein in the complex. Examples of the second antibodies include, without limitation, anti-BIM, anti-BID, anti-BAD, anti-BIK, anti-HRK, anti-BMF, anti-PUMA, anti-MCL-1, anti-BAX, anti-BAK antibodies.

The term “antibody” as used herein refers to an immunoglobulin or an antigen-binding fragment thereof, which can specifically bind to a target protein antigen. Antibodies can be identified and prepared by selection of antibodies from libraries of recombinant antibodies in phage or similar vectors, as well as preparation of polyclonal and monoclonal antibodies by immunizing animals such as rabbits or mice (see, e.g., Huse et al., Science (1989) 246:1275-1281; Ward et al, Nature (1989) 341:544-546).

It can be understood that in certain embodiments, the antibodies are modified or labeled to be properly used in various detection assays. In certain embodiments, the first antibody and/or the second antibody is detectably labeled.

In certain embodiments, the first antibody is conjugated with a first detectable label, and the second antibody is conjugated with a second detectable label, wherein the first detectable label and the second detectable label can permit generation a detectable signal when in close proximity. In certain embodiments, the first detectable label and the second detectable label comprise a pair of oligonucleotides. When the first antibody and the second antibody are close proximity, the pair of oligonucleotides are capable of interacting to enable enzymatic ligation to provide for a ligated product, which can be detection, by for example, amplification. Other detection systems such as time-resolved fluorescence, internal-reflection fluorescence, amplification (e.g., polymerase chain reaction) and Raman spectroscopy are also useful.

In certain embodiments, one of the first antibody and/or the second antibody is detectably labeled, and the other is capable of being captured. The antibody capable of being captured may comprise a capture moiety, or may be immobilized.

Examples of capture moiety can include for example binding partner or solid substrate, such as porous and non-porous materials, latex particles, magnetic particles, microparticles, strips, beads, membranes, microtiter wells and plastic tubes. The choice of solid phase material and method of detectably labeling the antigen or antibody reagent are determined based upon desired assay format performance characteristics. In certain embodiments, the antibody may be immobilized on a solid substrate. The immobilization can be via covalent linking or non-covalent attachment (e.g. coating).

The capture moiety can lead to capture of the antibody which is bound to the complex, thereby capturing the antibody-bound complex. By detecting the captured complex for the presence of the other antibody which specifically binds to the other partner in the complex and is detectably labeled (or is capable of specifically binding to a labeled ligand), the skilled person can detect or confirm the presence of the two partners in the complex, and thereby determining the level of the complex. In certain embodiments, the labeled ligand can comprise a secondary antibody that is linked to a detectable label. For example, the antibodies are linked to an electrochemiluminescence reagent when used in MSD assays.

In certain embodiments, the at least one reagent comprises a third reagent comprising a first oligonucleotide capable of hybridizing to the polynucleotide of MCL-1, or a third antibody capable of specifically binding to the protein of MCL-1. In certain embodiments, the at least one reagent further a fourth reagent comprising a second oligonucleotide capable of hybridizing to the polynucleotide of BCL-XL or BCL-2, or a fourth antibody capable of specifically binding to the protein of BCL-XL or BCL-2.

The measurement of level of MCL1, BCL-XL, and/or BCL-2 can be at RNA level, DNA level and/or protein level. Suitable reagents for detecting target RNA, target DNA or target proteins can be used. In certain embodiments, the first oligonucleotide and/or the second oligonucleotide comprise primers or probes that can hybridize to the polynucleotide of the at least one biomarker comprising MCL-1, BCL-XL, and/or BCL-2.

The term “primer” as used herein refers to oligonucleotides that can specifically hybridize to a target polynucleotide sequence, due to the sequence complementarity of at least part of the primer within a sequence of the target polynucleotide sequence. A primer can have a length of at least 8 nucleotides, typically 8 to 70 nucleotides, usually of 18 to 26 nucleotides. For proper hybridization to the target sequence, a primer can have at least 75%, at least 80%, at least 85%, at least 90%, or at least 95% sequence complementarity to the hybridized portion of the target polynucleotide sequence. Oligonucleotides useful as primers may be chemically synthesized according to the solid phase phosphoramidite triester method first described by Beaucage and Caruthers, Tetrahedron Letts. (1981) 22: 1859-1862, using an automated synthesizer, as described in Needham-Van Devanter et al, Nucleic Acids Res. (1984) 12:6159-6168.

Primers are useful in nucleic acid amplification reactions in which the primer is extended to produce a new strand of the polynucleotide. Primers can be readily designed by a skilled artisan using common knowledge known in the art, such that they can specifically anneal to the nucleotide sequence of the target nucleotide sequence of the at least one biomarker provided herein. Usually, the 3′ nucleotide of the primer is designed to be complementary to the target sequence at the corresponding nucleotide position, to provide optimal primer extension by a polymerase.

The term “probe” as used herein refers to oligonucleotides or analogs thereof that can specifically hybridize to a target polynucleotide sequence, due to the sequence complementarity of at least part of the probe within a sequence of the target polynucleotide sequence. Exemplary probes can be, for example DNA probes, RNA probes, or protein nucleic acid (PNA) probes. A probe can have a length of at least 8 nucleotides, typically 8 to 70 nucleotides, usually of 18 to 26 nucleotides. For proper hybridization to the target sequence, a probe can have at least 75%, at least 80%, at least 85%, at least 90%, or at least 95% sequence complementarity to hybridized portion of the target polynucleotide sequence. Probes and also be chemically synthesized according to the solid phase phosphoramidite triester method as described above. Methods for preparation of DNA and RNA probes, and the conditions for hybridization thereof to target nucleotide sequences, are described in Molecular Cloning: A Laboratory Manual, J. Sambrook et al., eds., 2nd edition. Cold Spring Harbor Laboratory Press, 1989, Chapters 10 and 11.

In certain embodiments, the primers or probes provided herein comprise a polynucleotide sequence hybridizable to a portion within the sequence of SEQ ID NO: 12, 18, 20, 2, 14, or 4. In certain embodiments, the primes or probes provided herein comprise a polynucleotide sequence having at least 60%, 65%, 70%, 75%, 80%, 85%, 90%, 95%, 97%, 98%, 99% or 100% complementarity to a portion within the sequence of SEQ ID NO: 12, 14, or 16.

In certain embodiments, the third antibody provided herein comprise an antigen-binding region capable of specifically binding to an epitope within the protein or polypeptide having the sequence of SEQ ID NO: 11, 17 or 19. In certain embodiments, the fourth antibody provided herein comprise an antigen-binding region capable of specifically binding to an epitope within the protein or polypeptide having the sequence of SEQ ID NO: 1, 13, or 3.

In certain embodiments, the at least one reagent (e.g. the primers, the probes and the antibodies provided herein) can be detectably labeled. In certain embodiments, the primers, the probes and the antibodies provided herein can specifically bind to a ligand which is detectably labeled.

In certain embodiments, the detectably labeled primers, probes or antibodies as provided herein can further comprise a quencher substance. A quencher substance refers to a substance which, when present in sufficiently close proximity to a fluorescent substance, can quench the fluorescence emitted by the fluorescent substance as a result of, for example, fluorescence resonance energy transfer (FRET).

Examples of a quencher substance include, without limitation, Tamra, Dabcyl, or Black Hole Quencher (BHQ, Biosearch Technologies), DDQ (Eurogentec), Iowa Black FQ (Integrated DNA Technologies), QSY-7 (Molecular Probes), and Eclipse quenchers (Epoch Biosciences).

Primer and probes can be labeled to high specific activity by either the nick translation method or by the random priming method. Useful probe labeling techniques are described in the literature (Fan, Y-S, Molecular cytogenetics: protocols and applications, Humana Press, Totowa, N.J. xiv, 411 (2002)).

The present disclosure also provides kits comprising the at least one reagent for measuring a level of the at least one biomarker, as provided herein.

Methods for Patient Identification, Treatment Guidance and Prognosis

In another aspect, the present disclosure in one aspect provides a method for identifying and/or selecting a subject having or suspected of having cancer for treatment with a BCL-2/BCL-XL dual inhibitor or a BCL-XL inhibitor or a BCL-2 inhibitor. In certain embodiments, the method comprises: measuring a test level of at least one biomarker comprising a first complex comprising BCL-XL or BCL-2 protein in a test sample obtained from the subject; comparing the level of the at least one biomarker with a corresponding reference level of the at least one biomarker to determine a difference; and determining that the subject is likely to respond to the treatment with the BCL-2/BCL-XL dual inhibitor or the BCL-XL inhibitor or a BCL-2 inhibitor when the difference reaches a threshold.

In another aspect, the present disclosure provides a method for treating cancer in a subject in need thereof. In certain embodiments, the method comprises: measuring a test level of at least one biomarker comprising a first complex comprising BCL-XL or BCL-2 protein in a test sample obtained from the subject; comparing the test level of the at least one biomarker with a corresponding reference level of the at least one biomarker to determine a difference; and administering a BCL-2/BCL-XL dual inhibitor or a BCL-XL inhibitor or a BCL-2 inhibitor to the subject when the difference reaches a threshold.

In another aspect, the present disclosure provides a method for monitoring therapeutic efficacy in a subject having cancer and having been treated with a BCL-2/BCL-XL dual inhibitor or a BCL-XL inhibitor or BCL-2 inhibitor for a therapeutic period. In certain embodiments, the method comprises: obtaining a test sample from the subject after the therapeutic period; measuring a level of at least one biomarker comprising a first complex comprising BCL-XL or BCL-2 in the sample to obtain a post-treatment level of the at least one biomarker; comparing the post-treatment level with a baseline level of the at least one biomarker measured on a test sample obtained from the subject before the therapeutic period, to determine post-treatment change in the level of the at least one biomarker, and recommending a treatment plan. For example, the treatment plan may involve, continuing administering the BCL-2/BCL-XL dual inhibitor or the BCL-XL or the BCL-2 inhibitor to the subject when the post-treatment change reaches a threshold. For another example, when the post-treatment change does not reach the threshold, the treatment plan may involve, increasing the dose or the dosing frequency of the BCL-2/BCL-XL dual inhibitor or the BCL-XL inhibitor or the BCL-2 inhibitor to the subject, administering a second anti-cancer therapeutic agent in combination to the BCL-2/BCL-XL dual inhibitor or the BCL-XL inhibitor or the BCL-2 inhibitor to the subject, or discontinuing administering the BCL-2/BCL-XL dual inhibitor or the BCL-XL inhibitor or the BCL-2 inhibitor to the subject.

In certain embodiments, the at least one biomarker further comprises a second complex comprising BCL-XL or BCL-2 protein.

In certain embodiments, the cancer is a solid tumor. In certain embodiments, the solid tumor is lung cancer, gastric cancer, esophageal cancer, colon cancer, cholangiocarcinoma, liver cancer, breast cancer, cervical cancer, ovarian cancer, head and neck cancer or brain tumors. In certain embodiments, the at least one biomarker for solid tumor comprises BCL-XL:BIM and BCL-XL:PUMA.

In certain embodiments, the cancer is a blood cancer. In certain embodiments, the blood cancer is chronic lymphocytic leukemia (CLL), acute myeloid leukemia (AML), multiple myeloma (MM), Waldenstrom macroglobulinemia (WM), acute lymphoblastic leukemia (ALL) or lymphoma. In certain embodiments, the at least one biomarker for a blood cancer comprises BCL-2:BIM and BCL-2:PUMA.

Sample Preparation

Any biological sample suitable for conducting the methods provided herein can be obtained from the subject. In certain embodiments, the sample contains a cell. In certain embodiments, the sample contains a cancer cell. In certain embodiments, the sample contains a non-cancer cell, for example Peripheral Blood Mononuclear Cells (PBMC). In certain embodiments, the test sample is a bodily fluid sample or a tissue sample.

In certain embodiments, the sample can be further processed by a desirable method for performing the measurement of the level of the at least one biomarker.

In certain embodiments, the method further comprises isolating or extracting cancer cell (such as circulating tumor cell) or PBMC from the biological fluid sample (such as peripheral blood sample) or the tissue sample obtained from the subject. The cancer cells can be separated by immunomagnetic separation technology such as that available from Immunicon (Huntingdon Valley, Pa.).

In certain embodiments, the method further comprises isolating or extracting PBMC from the biological fluid sample (such as peripheral blood sample).

In certain embodiments, the method further comprises extracting proteins from the sample. Protein extraction can involve lysing the cells and collecting cell lysate. For example, the isolated cells are resuspended in lysis buffer with protease phosphatase inhibitors and sonicated. The sonicated cells are centrifuged, and supernatant is collected for further analysis, e.g., for detecting the level of one or more biomarkers.

In certain embodiments, where RNA or DNA level of a biomarker is to be measured, the method further comprises isolating the nucleic acid from the sample. Various methods of extraction are suitable for isolating the DNA or RNA from cells or tissues, such as phenol and chloroform extraction, and various other methods as described in, for example, Ausubel et al., Current Protocols of Molecular Biology (1997) John Wiley & Sons, and Sambrook and Russell, Molecular Cloning: A Laboratory Manual 3^{rd} ed. (2001).

Commercially available kits can also be used to isolate RNA, including for example, the NucliSens extraction kit (Biomerieux, Marcy l'Etoile, France), QIAamp™ mini blood kit, Agencourt Genfind™, Rneasy® mini columns (Qiagen), PureLink® RNA mini kit (Thermo Fisher Scientific), and Eppendorf Phase Lock Gels™. A skilled person can readily extract or isolate RNA or DNA following the manufacturer's protocol.

In certain embodiments, a cell or tissue sample can be processed to perform in situ hybridization. For example, the tissue sample can be paraffin-embedded before fixing on a glass microscope slide, and then deparaffinized with a solvent, typically xylene.

Methods of Measuring Levels of the Protein Complex

The methods of the present disclosure include measuring the level of at least one biomarker comprising a first complex (and/or a second complex) described herein in a sample obtained from a subject having cancer or suspected of having cancer.

In certain embodiments, the first and/or the second complex comprises BCL-XL protein complexed with a BH3-only protein, BCL-2 protein complexed with a BH3-only protein, BCL-XL protein complexed with a BH3-containing protein, or BCL-XL protein complexed with a BH3-containing protein. In certain embodiment, the BH3-only protein is selected from the group consisting of: BIM, BID, BAD, BIK, HRK, BMF, and PUMA. In certain embodiment, the BH3-containing protein is BAX or BAK. In certain embodiments, the at least one biomarker comprises two or more complexes selected from the group consisting of: BCL-XL:BIM, BCL-XL:PUMA, BCL-2:BIM, BCL-2:PUMA, MCL-1:BIM, MCL-1:PUMA, and any combination thereof.

The level of the complex can be measured by any suitable assays known in the art for measuring protein-protein interaction, see, in general, Protein-Protein Interactions: A Molecular Cloning Manual, 2nd ed., Golemis and Adams, ed., Cold Spring Harbor Laboratory Press (2005)). In certain embodiments, the protein-protein interaction assay is based on immunoassay or proximity assays. Suitable methods generally for example, meso scale discovery (MSD) advanced enzyme-linked immunosorbent assay (MSD-ELISA), standard complex ELSIA, proximity ligation assay (PLA), co-immunoprecipitation, immunoblotting assay, or cross-linking assay, etc.

Immunoassays typically involves using antibodies that specifically bind to BCL-2 or BCL-XL in the complex, or to the BH3-only protein or BH3 domain containing protein, or to an epitope unique to the complex, to detect or measure the presence or level of the complex. Such antibodies can be obtained using methods known in the art (see, e.g., Huse et al., Science (1989) 246:1275-1281; Ward et al, Nature (1989) 341:544-546), or can be obtained from commercial sources. Examples of immunoassays include, without limitation, Western blotting, enzyme-linked immunosorbent assay (ELISA), enzyme immunoassay (EIA), radioimmunoassay (RIA), immunoprecipitations, sandwich assays, competitive assays, immunofluorescent staining and imaging, immunohistochemistry, and fluorescent activating cell sorting (FACS). For a review of immunological and immunoassay procedures, see Basic and Clinical Immunology (Stites & Terr eds., 7^{th} ed. 1991). Moreover, the immunoassays can be performed in any of several configurations, which are reviewed extensively in Enzyme Immunoassay (Maggio, ed., 1980); and Harlow & Lane, supra. For a review of the general immunoassays, see also Methods in Cell Biology: Antibodies in Cell Biology, volume 37 (Asai, ed. 1993); Basic and Clinical Immunology (Stites & Terr, eds., 7^{th} ed. 1991).

Co-immunoprecipitation is a popular assay for protein-protein interactions and protein complex detection. In general, a protein of interest is isolated (precipitated) from a lysate with a specific antibody to co-precipitate any partner protein that binds, directly or indirectly to the protein of interest and forms a complex. The precipitated complex is then analyzed, e.g., using western-blot with an antibody specifically binding to the partner protein.

MSD advanced ELISA is a method similar to enzyme-linked immunosorbent assay (ELISA) except MSD uses electrochemiluminescence (ECL) as a detection technique while ELISA uses a colormetric reaction. In general, a solution (e.g. cell lysate) containing the protein/complex of interest is added to a substrate (e.g., wells in a plate) coated with a capture antibody specifically binding to a protein of interest. After washing, a second antibody specifically binding to a partner protein that binds to the protein of interest is added. The second antibody (or an antibody binding to the second antibody) is linked to an ECL agent, e.g. a ruthenium (Ru) metal ion, and the substrate contains an electrode. In the presence of the partner protein/protein complex, the second antibody binds to the protein complex, and the ruthenium ion will be close enough proximity to the electrode to trigger an oxidation-reduction reaction that produces light which can be detected by a CCD camera. Compared to a traditional ELISA, MSD has many advantages such as higher sensitivity, better dynamic range, less matric effects, less sample required and better efficiencies. As a result, MSD can be used to detect a protein complex in cell lysate.

Proximity ligation assay (PLA) is an immunohistochemical method utilizing so called PLA probes for detection of protein-protein interaction or protein complex. Each PLA probe comes with a unique short DNA strand attached to it and bind either to species specific primary antibodies or consist of directly DNA-labeled primary antibodies. When the PLA probes are in close proximity, the DNA strands can interact through a subsequent addition of two other circle-forming DNA oligonucleotides. After joining of the two added oligonucleotides by enzymatic ligation, they are amplified via rolling circle amplification using a polymerase. The amplification reaction generates several hundred-fold replication of the DNA circle, which can be highlighted by fluorophore or enzyme labeled complementary oligonucleotide probes. The resulting high concentration of fluorescence or chromogenic signal in each single-molecule amplification product is easily visible as a distinct bright spot when viewed with either a fluorescence microscope or a standard bright field microscope.

While most protein-protein interactions are transient and may dissociate during sample preparation, cross-linking assay is an approach to stabilize or permanently adjoin the components of interaction of protein complexes. Once the components of a protein complex are covalently crosslinked, other steps (e.g., cell lysis, affinity purification, electrophoresis or mass spectrometry) can be used to analyze the protein-protein interaction while maintaining the original interacting complex. Homobifunctional, amine-reactive crosslinkers can be added to cells to crosslink potentially interacting proteins together, which can then be analyzed after lysis by western blotting. Crosslinkers can be membrane permeable, such as disuccinimidyl suberate (DSS), or crosslinking intracellular proteins, or they can be non-membrane permeable, such as bis-sulfosuccinimidyl suberate (BS3), or crosslinking cell-surface proteins. Furthermore, some crosslinkers can be cleaved by reducing agents, such as dithiobis-succinimidyl propionate (DSP) or 3,3′-dithiobis-succinimidyl propionate (DTSSP), to reverse the crosslinks. Alternatively, heterobifunctional crosslinkers that contain a photoactivatable group, such as (succinimidyl 4,4′-azipentanoate (SDA) product or Sulfo-SDA, can be used to capture transient interactions that may occur, such as after a particular stimulus. Photoactivation can also be also be after metabolic labeling with photoactivatable amino acids such as L-Photo-Leucine or L-Photo-Methionine. Crosslinking sites between proteins can be mapped by high precision using mass spectrometry, especially if a MS-cleavable crosslinker such as DSSO or DSBU is used.

In certain embodiments, the first and/or the second complex is a dominant complex in the sample. “Dominant” complex as used herein refers to the complex whose level in the sample is significant in the sample.

To determine the dominance of a particular complex, the level of a group of BCL-2 family protein complexes can be measured. In one embodiment, an overall level of the group of BCL-2 family protein complexes can be calculated, for example, as a sum, or a weighted sum. In such embodiment, the measured level of the particular complex can be compared with the overall level to obtain, for example a percentage of or a ratio to the overall level. If the percentage or the ratio of particular complex to the overall level exceed a certain value (e.g. 50%), then it is considered as one of the dominant complexes. In another embodiment, an average level of the group of complexes can be calculated, and the measured level of the particular complex can be compared with the average level to determine if it is above or below the average level, wherein if it is above, then it can be considered as one of the dominant complexes in the sample.

Methods of Measuring Levels of Additional Biomarker

In certain embodiments, the at least one biomarker further comprises MCL-1. Without wishing to be bound by any theory, it is believed that the level of MCL-1 can be indicative of potential resistance to the treatment. A high baseline level of MCL-1, or a significant increase in the level of MCL-1 after treatment, can be indicative of resistance.

In certain embodiments, the at least one biomarker further comprises BCL-XL or BCL-2. The amplification of BCL-XL or BCL-2 gene or proteins, can be indicative of response.

In certain embodiments, the methods provided herein further comprises measuring the level of the biomarker MCL-1, BCL-XL or BCL-2.

The biomarker MCL-1, BCL-XL or BCL-2 provided herein are intended to encompass different forms including mRNA, protein (and the complex thereof) and also DNA (e.g. genomic DNA). Therefore, the level of the at least one biomarker can be measured by, RNA level (e.g. mRNA level), protein level or DNA level. The mRNA level and/or the protein level can also be referred to as expression level of the at least one biomarker.

The RNA (e.g. mRNA) level or the DNA level of MCL-1, BCL-XL or BCL-2 can be measured by any suitable nucleic acid assays known in the art, for example, a nucleic acid amplification assay, a nucleic acid hybridization assay, a nucleic acid sequencing assay, and other methods such as high performance liquid chromatography (HPLC) fragment analysis, capillary electrophoresis, and the like. The protein level of MCL-1, BCL-XL or BCL-2 can be measured by any suitable assays such as immunoassays.

Amplification Assay

A nucleic acid amplification assay involves copying a target nucleic acid (e.g. DNA or RNA), thereby increasing the number of copies of the amplified nucleic acid sequence. Amplification may be exponential or linear. Exemplary nucleic acid amplification methods include, but are not limited to, amplification using the polymerase chain reaction (“PCR”, see U.S. Pat. Nos. 4,683,195 and 4,683,202; PCR Protocols: A Guide To Methods And Applications (Innis et al., eds, 1990)), reverse transcriptase polymerase chain reaction (RT-PCR), quantitative real-time PCR (qRT-PCR); quantitative PCR, such as TaqMan®, nested PCR, ligase chain reaction (See Abravaya, K., et al., Nucleic Acids Research, 23:675-682, (1995), branched DNA signal amplification (see, Urdea, M. S., et al., AIDS, 7 (suppl 2):S11-S14, (1993), amplifiable RNA reporters, Q-beta replication (see Lizardi et al., Biotechnology (1988) 6: 1197), transcription-based amplification (see, Kwoh et al., Proc. Natl. Acad. Sci. USA (1989) 86: 1173-1177), boomerang DNA amplification, strand displacement activation, cycling probe technology, self-sustained sequence replication (Guatelli et al., Proc. Natl. Acad. Sci. USA (1990) 87:1874-1878), rolling circle replication (U.S. Pat. No. 5,854,033), isothermal nucleic acid sequence based amplification (NASBA), and serial analysis of gene expression (SAGE).

In certain embodiments, the nucleic acid amplification assay is a PCR-based method.

In some embodiments, to measure the mRNA level of MCL-1, BCL-XL or BCL-2, the target RNA of MCL-1, BCL-XL or BCL-2 is reverse transcribed to cDNA before the amplification. Various reverse transcriptases may be used, including, but not limited to, MMLV RT, RNase H mutants of MMLV RT such as Superscript and Superscript II (Life Technologies, GIBCO BRL, Gaithersburg, Md.), AMV RT, and thermostable reverse transcriptase from Thermus thermophilus. For example, one method which may be used to convert RNA to cDNA is the protocol adapted from the Superscript II Preamplification system (Life Technologies, GIBCO BRL, Gaithersburg, Md.; catalog no. 18089-011), as described by Rashtchian, A., PCR Methods Applic., 4:S83-S91, (1994).

In certain embodiments, the level of MCL-1, BCL-XL or BCL-2 is quantified after the nucleic acid amplification assay. In certain embodiments, the level of MCL-1, BCL-XL or BCL-2 is quantified during the nucleic acid amplification assay, which is also known as real-time amplification or quantitative amplification.

Methods of quantitative amplification are disclosed in, e.g., U.S. Pat. Nos. 6,180,349; 6,033,854; and 5,972,602, as well as in, e.g., Gibson et al., Genome Research (1996) 6:995-1001; DeGraves, et al., Biotechniques (2003) 34(1): 106-10, 112-5; Deiman B, et al., Mol Biotechnol. (2002) 20(2): 163-79. Quantification is usually based on the monitoring of the detectable signal representing copies of the template in cycles of an amplification (e.g., PCR) reaction. Detectable signals can be generated by intercalating agents or labeled primer or labeled probes used during the amplification. Exemplary intercalating agents include SYBR GREEN™ and SYBR GOLD™.

In certain embodiments, a detectably labeled primer or a detectably labeled probe can be used, to allow detection or quantification of the biomarker corresponding to that primer or probe. In certain embodiments, the labeled primer or labeled probe comprise a detectable label comprising a fluorophore. In certain embodiments, the labeled primer or labeled probe may further comprise a quencher substance. Presence of both a fluorophore and a quencher substance in one primer or probe could be helpful to provide for a self-quenching probe such as a TaqMan (U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl., 4:357-362; Tyagi et al, 1996, Nature Biotechnology, 14:303-308; Nazarenko et al., 1997, Nucl. Acids Res., 25:2516-2521; U.S. Pat. Nos. 5,866,336 and 6,117,635). In an intact primer or probe, the quencher substance and the fluorophore are in close proximity, such that when the fluorophore is excited by irradiation, it transfers energy to the quencher substance in the same probe via fluorescence resonance energy transfer (FRET), thereby does not emit a signal. Such probes are useful in the 5′-3′ exonuclease “hydrolysis” PCR assay (also referred to as the TaqMan® assay) (see, U.S. Pat. Nos. 5,210,015 and 5,487,972; Holland et al., PNAS USA (1991) 88: 7276-7280; Lee et al, Nucleic Acids Res. (1993) 21: 3761-3766).

In a quantitative amplification assay (such as real-time PCR), levels of the detected biomarker can be quantified using methods known in the art. For example, during the amplification, the fluorescence signal can be monitored and calculated during each PCR cycle. The threshold cycle, or Ct value can be further calculated. Ct value is the cycle at which fluorescence intersects a predetermined value. The Ct can be correlated to the initial amount of nucleic acids or number of starting cells using a standard curve. A standard curve is constructed to correlate the differences between the Ct values and the logarithmic level of the measured biomarker.

As a quality control measure, level of an internal control biomarker may be measured. The skilled artisan will understand that an internal control biomarker can be inherently present in the sample and its level can be used to normalize the measured level of MCL-1, BCL-XL, or BCL-2, to offset any difference in the absolute amount of the sample.

Hybridization Assay

Nucleic acid hybridization assays use probes to hybridize to the target nucleic acid of MCL-1, BCL-XL, or BCL-2, thereby allowing detection of the target nucleic acid.

In certain embodiments, the probes for hybridization assay are detectably labeled. In certain embodiments, the nucleic acid-based probes for hybridization assay are unlabeled. Such unlabeled probes can be immobilized on a solid support such as a microarray, and can hybridize to the target nucleic acid molecules which are detectably labeled.

In certain embodiments, hybridization assays can be performed by isolating the nucleic acids (e.g. RNA or DNA), separating the nucleic acids (e.g. by gel electrophoresis) followed by transfer of the separated nucleic acid on suitable membrane filters (e.g. nitrocellulose filters), where the probes hybridize to the target nucleic acids and allows detection. See, for example, Molecular Cloning: A Laboratory Manual, J. Sambrook et al., eds., 2nd edition, Cold Spring Harbor Laboratory Press, 1989, Chapter 7. The hybridization of the probe and the target nucleic acid can be detected or measured by methods known in the art. For example, autoradiographic detection of hybridization can be performed by exposing hybridized filters to photographic film. Densitometric scanning of the photographic films exposed by the hybridized filters provides an accurate measurement of the target nucleic acid levels. Computer imaging systems can also be used to quantify the level of the biomarker.

In certain embodiments, hybridization assays can be in situ hybridization assay. In situ hybridization assay is useful to detect the presence of copy number variation (e.g. increase or amplification) at the locus of the biomarker of interest (e.g. MCL-1, BCL-XL or BCL-2). Probes useful for in situ hybridization assay can be locus specific probes, which hybridize to a specific locus on a chromosome to detect the presence or absence of a specific locus of interest (e.g. MCL-1, BCL-XL or BCL-2). Other types of probes may also be useful, for example, chromosome enumeration probes (e.g. hybridizable to a repeat sequence region in a chromosomal of interest to indicate presence or absence of the entire chromosome), and chromosome arm probes (e.g. hybridizable to a chromosomal region and indicate the presence or absence of an arm of a specific chromosome). Methods for use of unique sequence probes for in situ hybridization are described in U.S. Pat. No. 5,447,841, incorporated herein by reference. Probes can be viewed with a fluorescence microscope and an appropriate filter for each fluorophore, or by using dual or triple band-pass filter sets to observe multiple fluorophores. See, e.g., U.S. Pat. No. 5,776,688 to Bittner, et al., which is incorporated herein by reference. Any suitable microscopic imaging method can be used to visualize the hybridized probes, including automated digital imaging systems. Alternatively, techniques such as flow cytometry can be used to examine the hybridization pattern of the probes.

Sequencing Methods

Sequencing methods useful in the measurement of the level of biomarker of interest involves sequencing of the target nucleic acid and enumeration of the sequenced target nucleic acid. Examples of sequence methods include, without limitation, RNA sequencing, pyrosequencing, and high throughput sequencing.

High throughput sequencing involves sequencing-by-synthesis, sequencing-by-ligation, and ultra-deep sequencing (such as described in Marguiles et al., Nature 437 (7057): 376-80 (2005)). Sequence-by-synthesis involves synthesizing a complementary strand of the target nucleic acid by incorporating labeled nucleotide or nucleotide analog in a polymerase amplification. Immediately after or upon successful incorporation of a label nucleotide, a signal of the label is measured and the identity of the nucleotide is recorded. The detectable label on the incorporated nucleotide is removed before the incorporation, detection and identification steps are repeated. Examples of sequence-by-synthesis methods are known in the art, and are described for example in U.S. Pat. Nos. 7,056,676, 8,802,368 and 7,169,560, the contents of which are incorporated herein by reference. Sequencing-by-synthesis may be performed on a solid surface (or a microarray or a chip) using fold-back PCR and anchored primers. Target nucleic acid fragments can be attached to the solid surface by hybridizing to the anchored primers, and bridge amplified. This technology is used, for example, in the Illumina® sequencing platform.

Pyrosequencing involves hybridizing the target nucleic acid regions to a primer and extending the new strand by sequentially incorporating deoxynucleotide triphosphates corresponding to the bases A, C, G, and T (U) in the presence of a polymerase. Each base incorporation is accompanied by release of pyrophosphate, converted to ATP by sulfurylase, which drives synthesis of oxyluciferin and the release of visible light. Since pyrophosphate release is equimolar with the number of incorporated bases, the light given off is proportional to the number of nucleotides adding in any one step. The process is repeated until the entire sequence is determined.

In certain embodiments, the level of the biomarkers described herein is measured by whole transcriptome shotgun sequencing (RNA sequencing). The method of RNA sequencing has been described (see Wang Z, Gerstein M and Snyder M, Nature Review Genetics (2009) 10:57-63; Maher C A et al., Nature (2009) 458:97-101; Kukurba K & Montgomery S B, Cold Spring Harbor Protocols (2015) 2015(11): 951-969).

Immunoassays

Immunoassays typically involves using antibodies that specifically bind to the biomarkers. Such antibodies can be obtained using methods known in the art (see, e.g., Huse et al., Science (1989) 246:1275-1281; Ward et al, Nature (1989) 341:544-546), or can be obtained from commercial sources. Examples of immunoassays include, without limitation, Western blotting, enzyme-linked immunosorbent assay (ELISA), enzyme immunoassay (EIA), radioimmunoassay (RIA), immunoprecipitations, sandwich assays, competitive assays, immunofluorescent staining and imaging, immunohistochemistry, and fluorescent activating cell sorting (FACS). For a review of immunological and immunoassay procedures, see Basic and Clinical Immunology (Stites & Terr eds., 7^{th} ed. 1991). Moreover, the immunoassays can be performed in any of several configurations, which are reviewed extensively in Enzyme Immunoassay (Maggio, ed., 1980); and Harlow & Lane, supra. For a review of the general immunoassays, see also Methods in Cell Biology: Antibodies in Cell Biology, volume 37 (Asai, ed. 1993); Basic and Clinical Immunology (Stites & Terr, eds., 7^{th} ed. 1991).

Any of the assays and methods provided herein for the measurement of the level of the biomarker can be adapted or optimized for use in automated and semi-automated systems, or point of care assay systems.

The level of each of the biomarkers described herein can be normalized using a proper method known in the art. For example, the level of the biomarker can be normalized to a standard level of a standard marker, which can be predetermined, determined concurrently, or determined after a sample is obtained from the subject. The standard marker can be run in the same assay or can be a known standard marker from a previous assay. For another example, the level of the biomarker can be normalized to an internal control which can be an internal marker, or an average level or a total level of a plurality of internal markers.

Comparing with a Corresponding Reference Level

The test level measured for the at least one biomarker (e.g. optionally normalized) can be compared with a corresponding reference level of the corresponding biomarker to determine a difference.

The term “reference level” of the at least one biomarker (e.g. the complex provided herein) as used herein refers to a level of the biomarker that is representative of an average cancer or tumor sample. The reference level can be a typical level, a measured level, an average level or a range of the level of the corresponding biomarker that would normally be observed in one or more samples, or expected to be observed in a sample comparable to an average cancer or tumor sample. In certain embodiments, the reference level is an average of the level of the biomarker in a comparable tumor sample (e.g. of the same tumor type). In certain embodiments, the reference level is an average level of the at least one biomarker in representative samples of the same type of cancer. In certain embodiments, the reference level is a representative level of the at least one biomarker in an average tumor sample. For example, it can be an empirical level of the biomarker that is considered to be representative of in a comparable cancer sample or in cancer in general. In certain embodiments, the reference level of the at least one biomarker is obtained using the same or comparable measurement method or assay as used in the measurement of the level of the biomarker in the test sample.

In certain embodiments, the reference level can be predetermined. For example, the reference level can be calculated or generalized based on measurements of the biomarker level in a collection of general cancer or tumor samples or tissues from a tumor of the same type, or from blood cancer. For another example, the reference level can be based on statistics of the level of the biomarkers generally observed in an average cancer or tumor samples from a general cancer or tumor population. In certain embodiments, the reference level is calculated or generated by an algorithm considering a plurality of actual tumor samples and the actual measured levels of the biomarker in these samples.

In certain embodiments, the comparing step in the method provided herein is performed with an algorithm. In certain embodiments, the algorithm is a classification algorithm.

Examples of classification algorithm include, such as partial least square (Wold S et al., PLS for Multivariate Linear Modeling, In H van de Waterbeemd (ed.), Chemometric Methods in Molecular Design, pp. 195-218. VCH, Weinheim), elastic net (Zou H et al., Journal of the Royal Statistical Society, Series B (2005) 67(2): 301-320), support vector machine (Vapnik V), random forest (Breiman, L. (2001). Random Forests, Machine Learning 45(1), 5-32. See also Breiman, L (2002), “Manual On Setting Up, Using, And Understanding Random Forests V3.1.), neural net (Bishop C, Neural Networks for Pattern Recognition (1995) Oxford University Press, Oxford) and gradient boosting machine (Friedman J, Greedy Function Approximation: A Gradient Boosting Machine, Annals of Statistics (2001) 29(5), 1189-1232). A classification algorithm allows a computer system to recognize the pattern of a dataset, and based on such recognized pattern, group the dataset to a particular category to which dataset belongs.

In certain embodiments, the classification algorithm is trained with a training set of data containing datasets obtained from multiple samples, each of which has been categorized to either responsiveness or non-responsiveness to the BCL-2/BCL-X1 dual inhibitors or BCL-XL inhibitor or BCL-2 inhibitor based on clinical observation. Each dataset contains the measured level of the at least one biomarker comprising the at least one biomarker for a sample obtained from a subject. By designating the training particular datasets to the known categories, a discriminant for classification is determined. For new dataset yet to be classified, it is assigned to the discriminant which enables prediction of the outcome of the grouping of the new dataset.

In certain embodiments, the classification algorithm can transform the predicted outcome of each dataset into a score. For example, the classification algorithm can transform the predicted outcome of the dataset for the test sample into a test score, and the datasets for reference samples into a reference score. The difference between the test score (representing the test level) and the reference score (representing the reference level) can be determined, wherein the test score and the reference score are calculated by the algorithm. A threshold can be determined to allow discrimination of a responsive subject from a non-responsive subject. If the difference reaches the threshold, then the subject can be classified as a responsive subject.

In certain embodiments, the comparing step in the method provided herein involves determining the difference between the test level and the reference level. The difference from the reference level can be elevation or reduction. Depending on the specific biomarker, one may seek elevation in one biomarker but reduction in another biomarker, in order to predict responsiveness to the BCL-2/BCL-X1 dual inhibitors or the BCL-X1 inhibitors according to the methods provided herein. The term “elevation” as used herein refers to levels of a biomarker as measured in the test sample is higher than the corresponding reference level of that biomarker. Similarly, “reduction” as used herein refer to levels of a biomarker as measured in the sample is lower than the corresponding reference level of that biomarker. The term “maintenance” as used herein refers to no significant change.

In certain embodiments, an elevation in level of the first and/or second complex comprising BCL-2 or BCL-XL is relevant to responsiveness to the BCL-2/BCL-XL dual inhibitors provided herein. In certain embodiments, when levels of two or more complexes are measured, the respective measured level of each of the complexes are combined to obtain the level of the at least one biomarker. For example, the first level of the first complex is combined with the second level of the second complex to provide for the measured level of the at least one biomarker.

In certain embodiments, the at least one biomarker comprises a combination of BCL-XL:BIM and BCL-XL:PUMA; or a combination of BCL-2:BIM and BCL-2:PUMA. In certain embodiments, the at least one biomarker comprises a combination of BCL-2:BIM, BCL-2:PUMA, BCL-XL:BIM, and BCL-XL:PUMA.

In certain embodiments, levels of one or more additional biomarkers (such as MCL-1, BCL-XL, BCL-2) can be further considered, for example, to improve prognosis sensitivity.

In certain embodiments, an elevation in level of complex comprising BCL-2 or BCL-XL, accompanied by normal to reduction in level of MCL-1, is relevant to responsiveness to the BCL-2/BCL-XL dual inhibitors provided herein. In certain embodiments, elevation in the combined levels (i.e. sum) of BCL-XL protein and BCL-2 protein may also be relevant to responsiveness to the BCL-2/BCL-XL dual inhibitors provided herein.

In certain embodiments, an elevation in level of complex comprising BCL-2, accompanied by normal to reduction in level of MCL-1, is relevant to responsiveness to the BCL-2 inhibitors provided herein.

In certain embodiments, an elevation in level of complex comprising BCL-XL, accompanied by normal to reduction in level of MCL-1, is relevant to responsiveness to the BCL-XL inhibitors provided herein.

In certain embodiments, the difference from the reference level is further compared with a threshold. In certain embodiments, a threshold can be set by statistical methods, such that if the difference from the reference level reaches the threshold, such difference can be considered statistically significant. Useful statistical analysis methods are described in L. D. Fisher & G. vanBelle, Biostatistics: A Methodology for the Health Sciences (Wiley-Interscience, NY, 1993). Statistically significance can be determined based on confidence (“p”) values, which can be calculated using an unpaired 2-tailed t test. A p value less than or equal to, for example, 0.1, 0.05, 0.025, or 0.01 usually can be used to indicated statistical significance. Confidence intervals and p-values can be determined by methods well-known in the art. See, e.g., Dowdy and Wearden, Statistics for Research, John Wiley & Sons, New York, 1983.

In certain embodiments, the threshold is reached when the test level of the combination of BCL-2:BIM, BCL-2:PUMA, BCL-XL:BIM, and BCL-XL:PUMA is at least 2-fold above the reference level.

In certain embodiments, the threshold for the level of additional biomarkers such as MCL-1, BCL-XL, or BCL-2 can be further determined. In certain embodiments, the threshold is reached when the test level of MCL-1 is no more than 100% of the reference level of MCL-1.

In certain embodiments, the threshold for the level of BCL-XL is at least 50%, at least 80%, at least 100%, at least 110%, at least 120%, at least 130%, at least 150%, at least 200%, or at least 250% higher than the reference level of BCL-XL. For example, the threshold for the level of BCL-2 is at least 50%, at least 80%, at least 100%, at least 110%, at least 120%, at least 130%, at least 150%, at least 200%, or at least 250% higher than the reference level of BCL-2.

Method of Predicting Responsiveness, Guiding Treatment

To identify and/or select a subject having or suspected of having cancer for treatment with a BCL-2/BCL-XL dual inhibitor or BCL-XL inhibitor, or BCL-2 inhibitor, the level of the at least one biomarker comprising the complex comprising BCL-2 or BCL-XL can be measured before the treatment and compared with the reference level. If the difference reaches a threshold, then the subject is identified as likely to respond to the treatment with the BCL-2/BCL-XL dual inhibitor or BCL-2 inhibitor or BCL-XL inhibitor. In certain embodiments, the identified or selected responsive subject is administered with a therapeutically effective amount of the BCL-2/BCL-XL dual inhibitor or BCL-2 inhibitor or BCL-XL inhibitor provided herein.

In certain embodiments, if the difference does not reach a threshold, then the subject is identified as less likely to respond to the treatment with the BCL-2/BCL-xL dual inhibitor. These identified subjects may be recommended to take additional tests to confirm the conclusion, or alternatively may be recommended not to be treated with the BCL-2/BCL-XL dual inhibitor or BCL-2 inhibitor or BCL-XL inhibitor provided herein.

In another aspect, the methods provided herein are for monitoring therapeutic efficacy in a subject having cancer and having been treated with a BCL-2/BCL-XL dual inhibitor or BCL-XL inhibitor or BCL-2 inhibitor for a therapeutic period. In certain embodiments, the level of the at least one biomarker can be measured before the therapeutic period to establish a baseline level of the biomarker. After a certain treatment period, the level (“post-treatment level”) of the at least one biomarker can be measured in a test sample newly obtained from the subject after the treatment, and difference (“post-treatment difference”) from the reference level is determined. A post-treatment change in the level of the at least one biomarker can be determined. If the post-treatment change reaches the threshold, then the subject is identified as being responsive to the treatment with the BCL-2/BCL-xL dual inhibitor or BCL-XL inhibitor or BCL-2 inhibitor. Alternatively, if the post-treatment change does not reach the threshold, then the subject is identified as having reduced responsiveness or no longer responsive to the BCL-2/BCL-XL dual inhibitor or BCL-XL inhibitor or BCL-2 inhibitor provided herein.

In certain embodiments, the methods for treating cancer in a subject comprises treating the cells in the sample obtained from the subject with a BCL-2/BCL-xL dual inhibitor or BCL-XL inhibitor or BCL-2 inhibitor, and determining the post-treatment change of the at least one biomarker in the sample. Such a post-treatment change could be useful for identifying and/or selecting a subject for the treatment. In certain embodiments, the method for identifying and/or selecting a subject having cancer for treatment with a BCL-2/BCL-XL dual inhibitor or a BCL-XL or BCL-2 inhibitor, comprises: (a) measuring a baseline level of at least one biomarker comprising a first complex comprising BCL-XL or BCL-2 protein, in a test sample comprising a cell obtained from the subject; (b) treating the test sample with a BCL-2/BCL-XL dual inhibitor or a BCL-XL inhibitor or a BCL-2 inhibitor, (c) measuring a post-treatment level of at least one biomarker in the treated test sample; (d) comparing the post-treatment level with the baseline level of the at least one biomarker to determine post-treatment change in the level of the at least one biomarker; and (e) determining that the subject is likely to respond to the treatment with the BCL-2/BCL-XL dual inhibitor or the BCL-XL inhibitor or the BCL-2 inhibitor when the post-treatment change reaches a threshold.

In certain embodiments, the method for treating cancer in a subject in need thereof, comprises: (a) measuring a baseline level of at least one biomarker comprising a first complex comprising BCL-XL or BCL-2 protein, in a test sample comprising a cell obtained from the subject; (b) treating the test sample with a BCL-2/BCL-XL dual inhibitor or a BCL-XL inhibitor or a BCL-2 inhibitor, (c) measuring a post-treatment level of at least one biomarker in the treated test sample; (d) comparing the post-treatment level with the baseline level of the at least one biomarker to determine post-treatment change in the level of the at least one biomarker; and (e) administering a BCL-2/BCL-XL dual inhibitor or a BCL-XL inhibitor or a BCL-2 inhibitor to the subject when the post-treatment change reaches a threshold.

In certain embodiments, the threshold is reached when the post-treatment change is at least 2-fold reduction.

BCL-2/BCL-CL Dual Inhibitors or BCL-XL or BCL-2 Inhibitors

In certain embodiments, the BCL-2/BCL-xL dual inhibitor or BCL-XL or BCL-2 inhibitor described herein has a structural formula (I), (II), or (III):

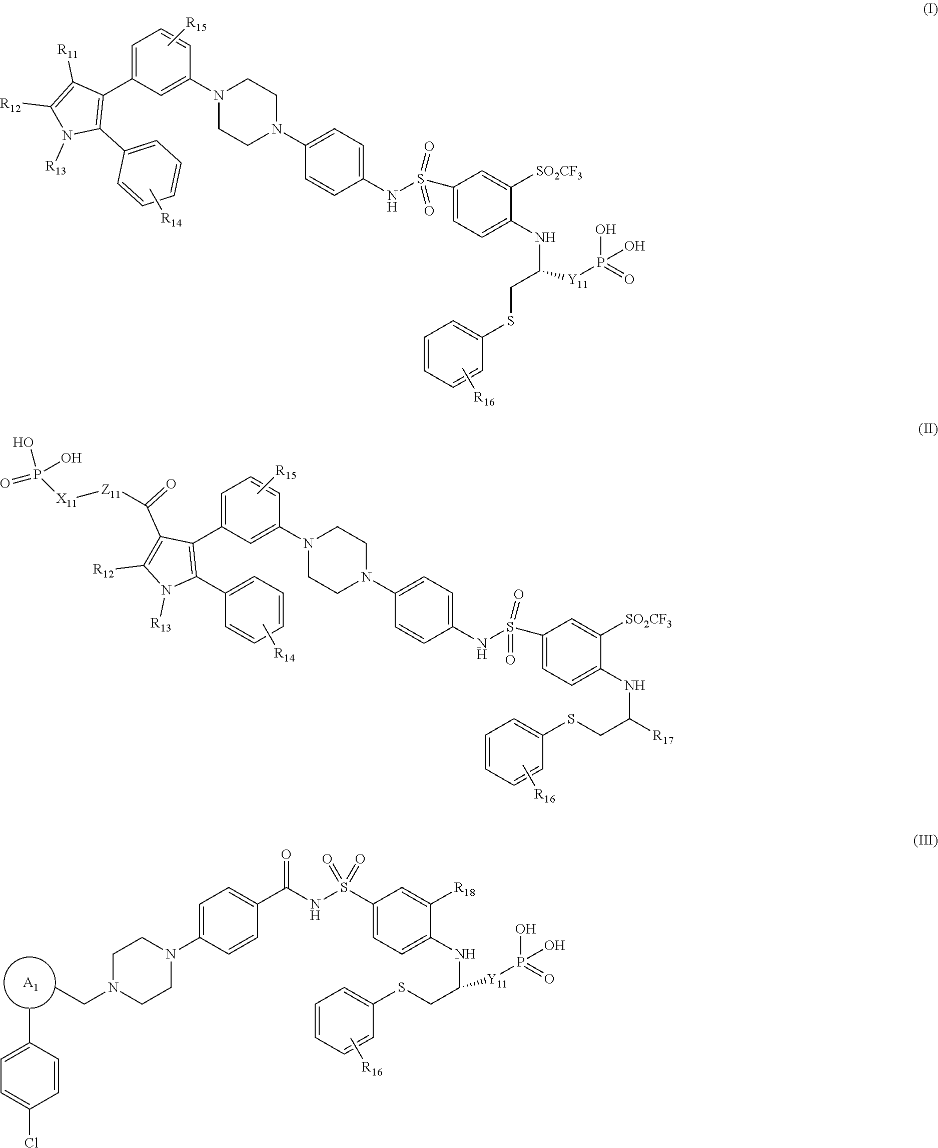

wherein the A₁ ring is

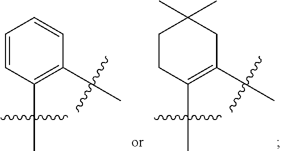

X₁₁, substituted or unsubstituted, is selected from the group consisting of alkylene, alkenylene, cycloalkylene, cycloalkenylene, and heterocycloalkylene;

Y₁₁ is selected from the group consisting of (CH₂)ₙ—N(R₁₁ₐ) and

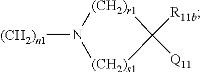

Q₁₁ is selected from the group consisting of O, O(CH₂)₁₋₃, NR_{11c}, NR_{11c} (C₁₋₃alkylene), OC(═O)(C₁₋₃alkylene), C(═O)O, C(═O)O(C₁₋₃alkylene), NHC(═O)(C₁₋₃alkylene), C(═O)NH, and C(═O)NH(C₁₋₃alkylene);

Z₁₁ is O or NR_{11c}

R₁₁ and R₁₂, independently, are selected from the group consisting of H, CN, NO₂, halo, alkyl, cycloalkyl, alkenyl, cycloalkenyl, alkynyl, aryl, heteroaryl, heterocycloalkyl, OR₁′, SR₁′, NR₁′R₁″, COR₁′, CO₂R₁′, OCOR₁′, CONR₁′R₁″, CONR₁′SO₂R₁″, NR₁“COR₁”, NR₁′CONR₁″R₁′″, NR₁′C═SNR₁″R₁′″, NR₁′SO₂R₁″, SO₂R₁′, and SO₂NR₁′R₁″;

R₁₃ is selected from a group consisting of H, alkyl, cycloalkyl, alkenyl, cycloalkenyl, alkynyl, aryl, heteroaryl, heterocycloalkyl, OR₁′, NR₁′R₁″, OCOR₁′, CO₂R₁′, COR₁′, CONR₁′R₁″, CONR₁′SO₂R₁″, C₁₋₃alkyleneCH(OH)CH₂OH, SO₂R₁′, and SO₂NR₁′R₁″;

R₁′, R₁″, and R₁′″, independently, are H, alkyl, cycloalkyl, alkenyl, cycloalkenyl, alkynyl, aryl, heteroaryl, C₁-3alkyleneheterocycloalkyl, or heterocycloalkyl;

R₁′ and R₁″, or R₁″ and R₁′″, can be taken together with the atom to which they are bound to form a 3 to 7 membered ring;

R₁₄ is hydrogen, halo, C₁₋₃alkyl, CF₃, or CN;

R₁₅ is hydrogen, halo, C₁₋₃alkyl, substituted C₁₋₃alkyl, hydroxyalkyl, alkoxy, or substituted alkoxy;

R₁₆ is selected from the group consisting of H, CN, NO₂, halo, alkyl, cycloalkyl, alkenyl, cycloalkenyl, alkynyl, aryl, heteroaryl, heterocycloalkyl, OR₁′, SR₁′, NR₁′R₁″, CO₂R₁′, OCOR₁′, CONR₁′R₁″, CONR₁“SO₂R₁”, NR₁′COR₁″, NR₁′CONR₁″R₁′″, NR₁′C═SNR₁″R₁′″, NR₁'SO₂R₁″, SO₂R₁′, and SO₂NR₁′R₁″;

R₁₇, substituted or unsubstituted, is selected form the group consisting of hydrogen, alkyl, alkenyl, (CH₂)₀₋₃cycloalkyl, (CH₂)₀₋₃cycloalkenyl, (CH₂)₀₋₃heterocycloalkyl, (CH₂)₀₋₃aryl, and (CH₂)₀₋₃heteroaryl;

R₁₈ is selected form the group consisting of hydrogen, halo, NO₂, CN, CF₃SO₂, and CF₃;

R₁₁ₐ is selected from the group consisting of hydrogen, alkyl, heteroalkyl, alkenyl, hydroxyalkyl, alkoxy, substituted alkoxy, cycloalkyl, cycloalkenyl, and heterocycloalkyl;

R_{11b} is hydrogen or alkyl;

R_{11c} is selected from the group consisting of hydrogen, alkyl, substituted alkyl, hydroxyalkyl, alkoxy, and substituted alkoxy; and

n₁, r₁, and s₁, independently, are 1, 2, 3, 4, 5, or 6;

or a pharmaceutically acceptable salt of (I), (II), or (III).

In certain embodiments, Y11 is

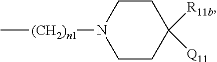

n is an integer of 1-3, R_{11b} is hydrogen or C₁₋₃ alkyl, Q is O, O(CH₂)₁₋₃, C(═O)O(CH₂)₁₋₃, OC(═O)(CH₂)₁₋₃ or C(═O)O(C₃H₇)₁₋₃.

In certain embodiments, the BCL-2/BCL-xL dual inhibitor or BCL-XL inhibitor or BCL-2 inhibitor described herein having a structural formula (I), (II) or (III) is selected from the group consisting of:

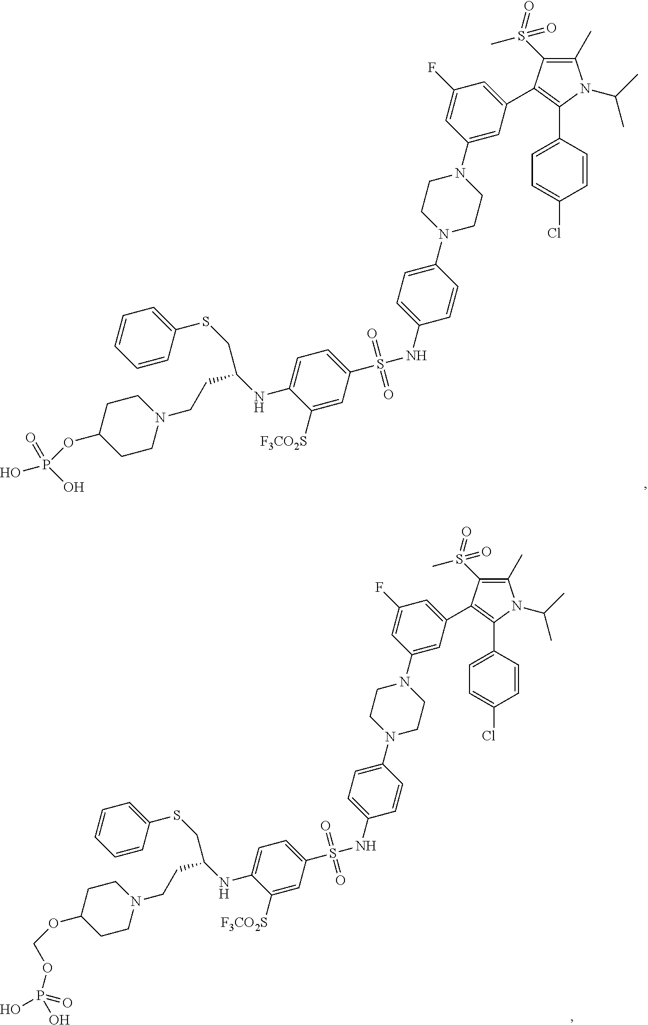

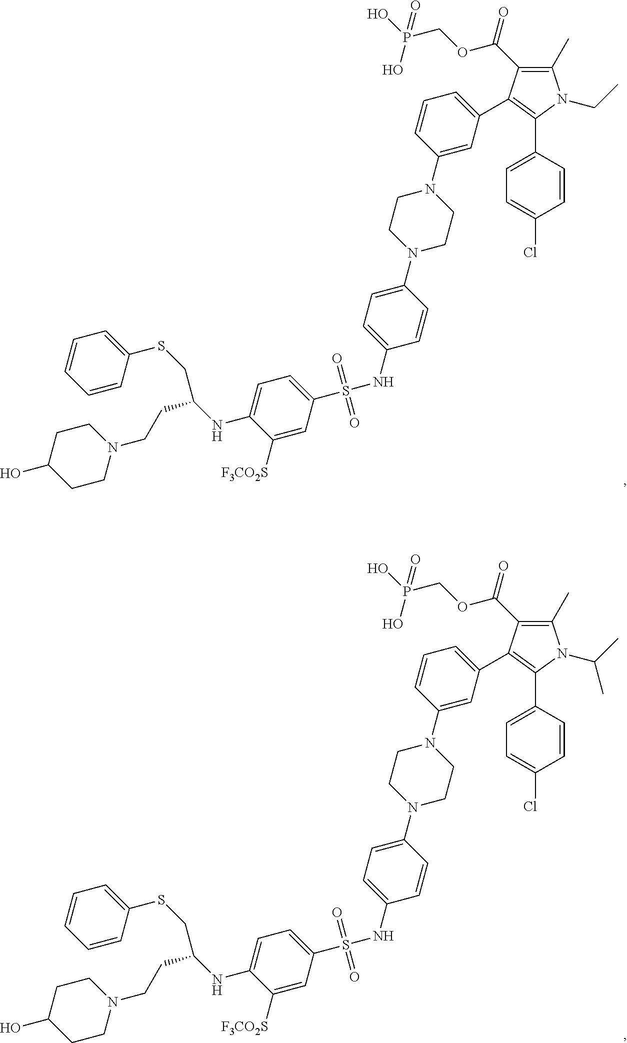

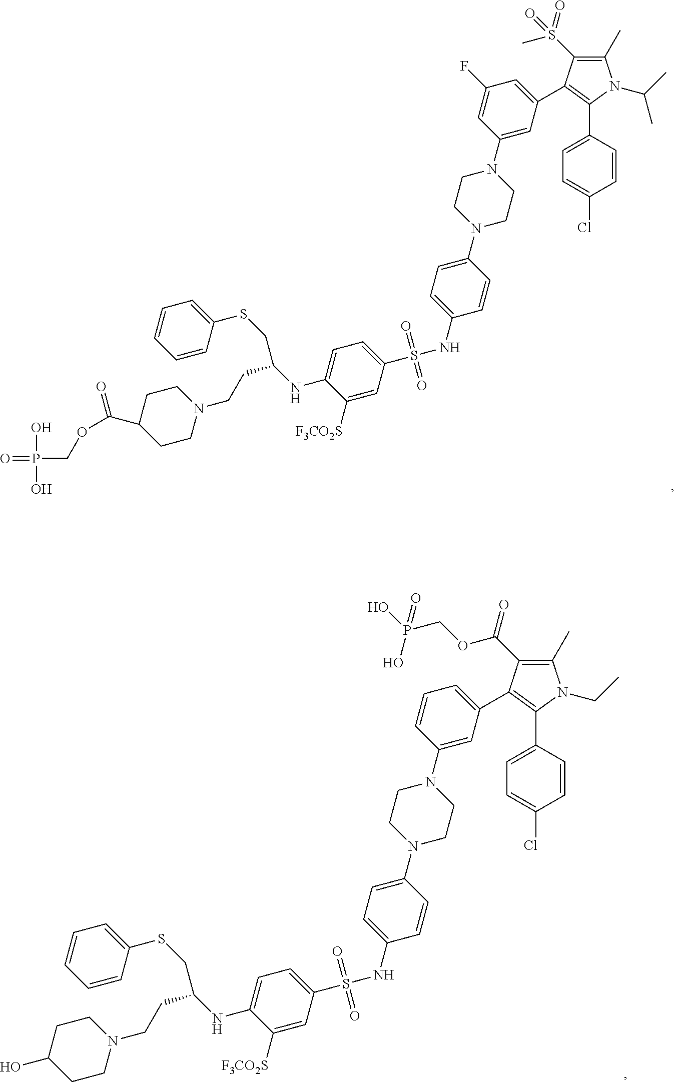

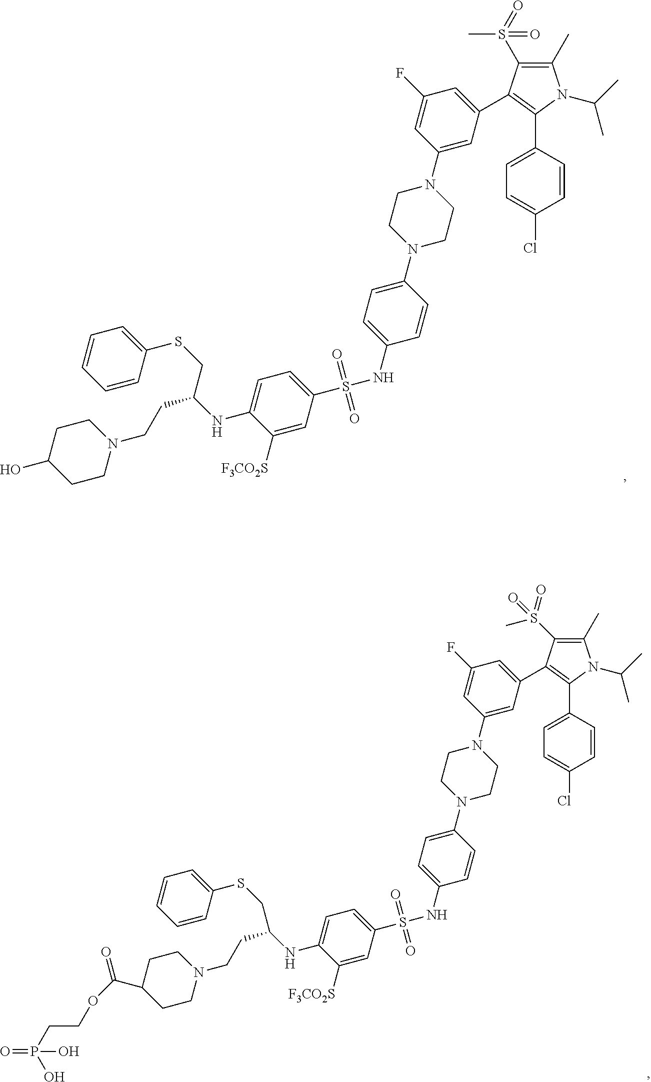

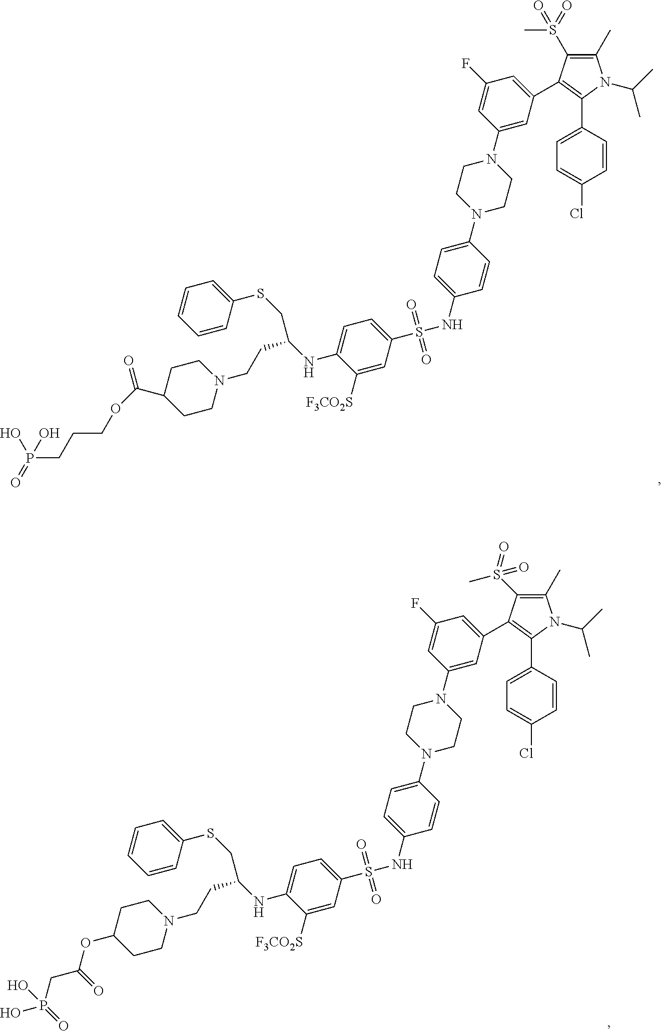

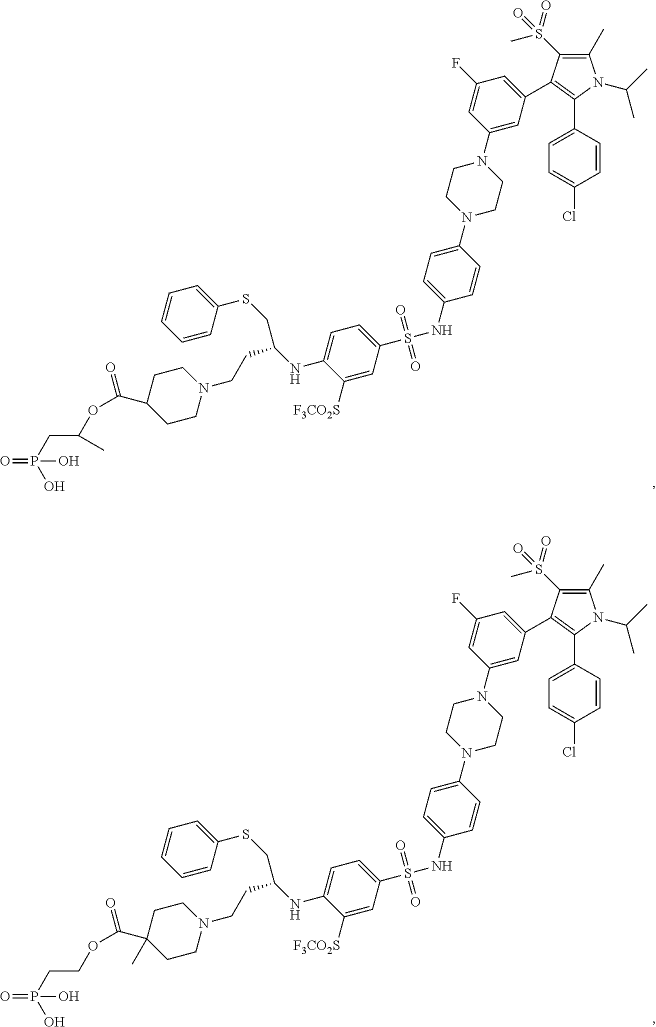

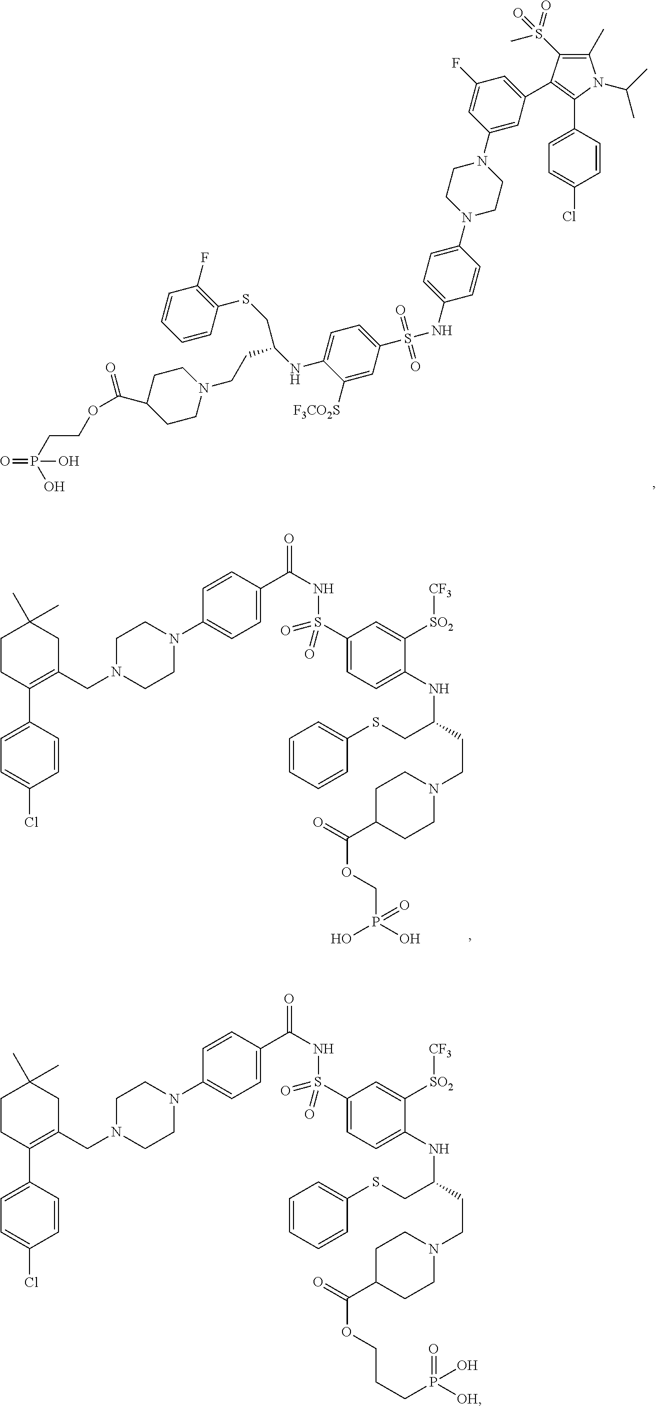

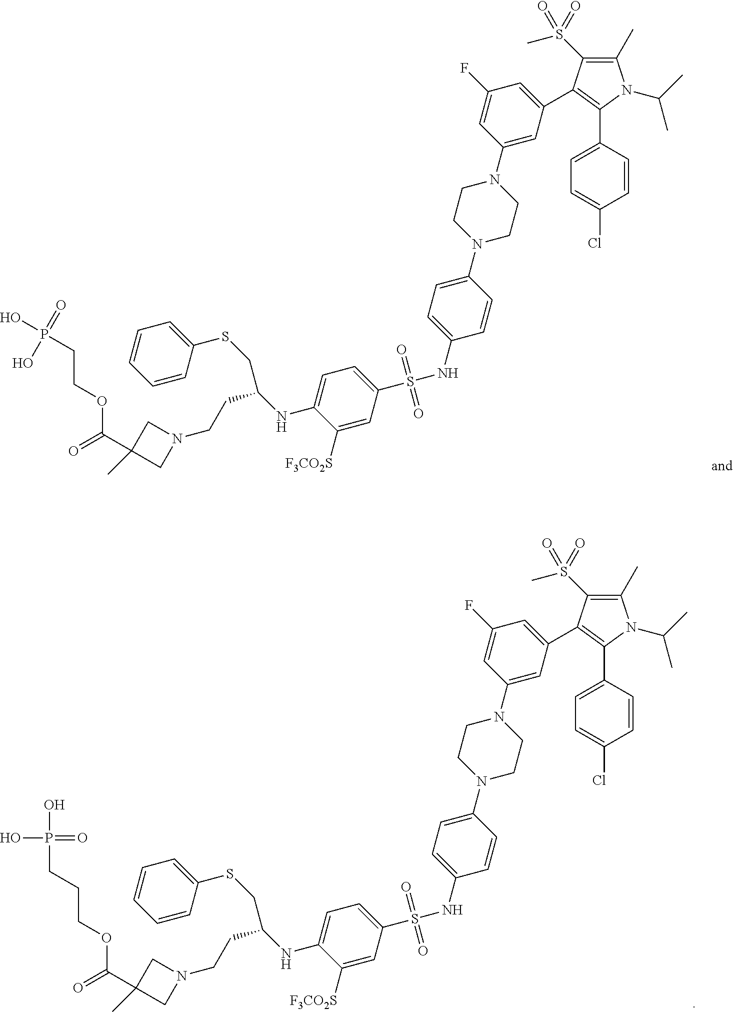

In certain embodiments, the BCL-2/BCL-xL dual inhibitor is (R)-2-(1-(3-(4-(N-(4-(4-(3-(2-(4-chlorophenyl)-1-isopropyl-5-methyl-4-(methylsulfonyl)-1H-pyrrol-3-yl)-5-fluorophenyl)piperazin-1-yl)phenyl)sulfamoyl)-2-(trifluoromethylsulfonyl)phenylamino)-4-(phenylthiol)butyl)piperidine-4-carbonyloxy)ethylphosphonic acid or a pharmaceutically acceptable salt thereof (also referred to as “Compound A” herein).

Compound A is a small-molecule compound that binds to BCL-2, BCL-xL and BCL-w proteins with very high affinities with IC₅₀ values of 1.6 nM, 4.4 nM, and 9.3 nM, respectively. Compound A has a weak affinity to Mcl-1 protein. Compound A demonstrates potent cell growth inhibitory activity in vitro with nanomolar potencies in a subset of cancer cell lines. Mechanistically, Compound A effectively induces cleavage of caspase-3 and PARP, biochemical markers of apoptosis of human cancers in cancer cells and in xenograft tumor tissues.

In certain embodiments, the BCL-2/BCL-xL dual inhibitor or BCL-XL inhibitor or BCL-2 inhibitor described herein has a structural formula (IV):

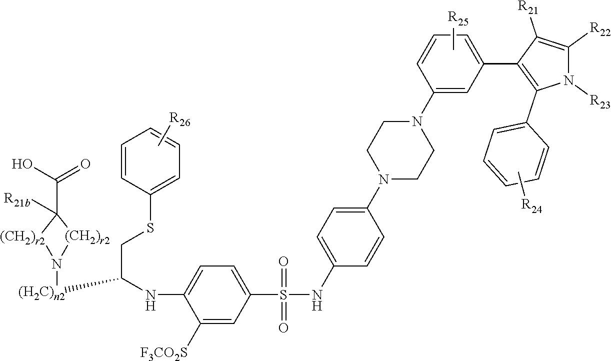

wherein,

R₂₁ is SO₂R₂′,

R₂₂ is alkyl, preferably C₁-C₄ alkyl, more preferably methyl, propyl, or isopropyl,

R₂₃ is alkyl, preferably C₁-C₄ alkyl, more preferably methyl, propyl, or isopropyl,

R₂₄ is halogen, preferably fluoride, chloride,

R₂₅ is halogen, preferably fluoride, chloride,

R₂₆ is selected from H, halogen, alkyl, preferably fluoride, chloride, C₁-C₄ alkyl, more preferably methyl, propyl, isopropyl

R_{21b} is H or alkyl, preferably C₁-C₄ alkyl, more preferably methyl, propyl, or isopropyl,

n₂, r₂ and s₂ are independently 1, 2, 3, 4, 5 or 6, more preferably, r₂ and s₂ are both 2 and n₂ is 3, 4 or 5, more preferably, all of n₂, r₂ and s₂ are 2, and

R₂′ is alkyl, preferably C₁-C₄ alkyl, more preferably methyl, propyl, or isopropyl.

In certain embodiments, the BCL-2/BCL-xL dual inhibitor or BCL-XL inhibitor or BCL-2 inhibitor described herein having a structural formula (IV) are selected from the group consisting of:

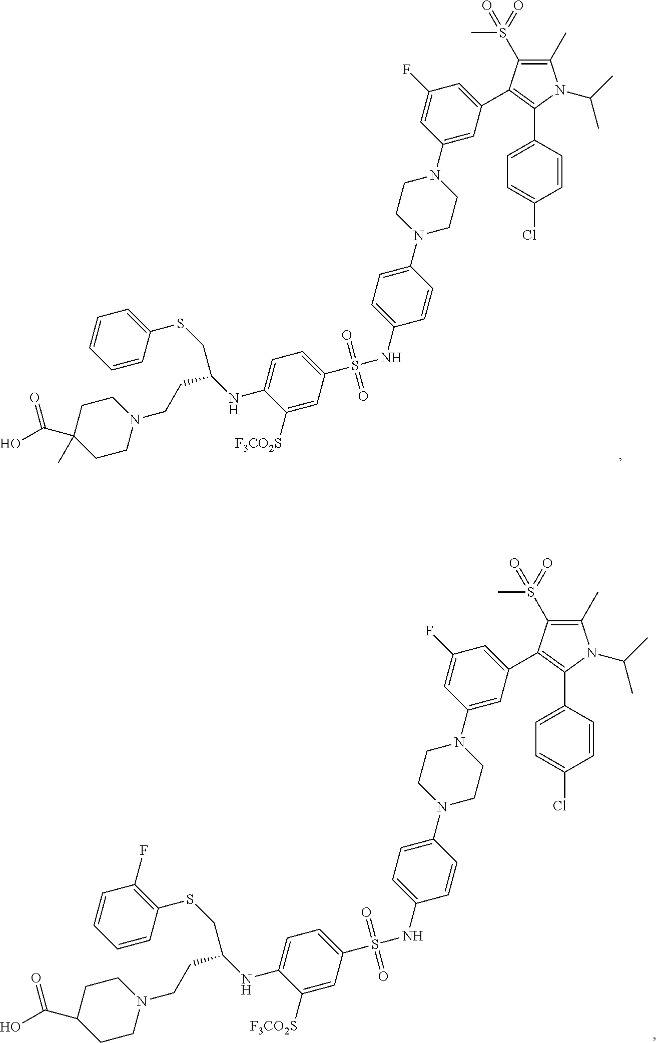

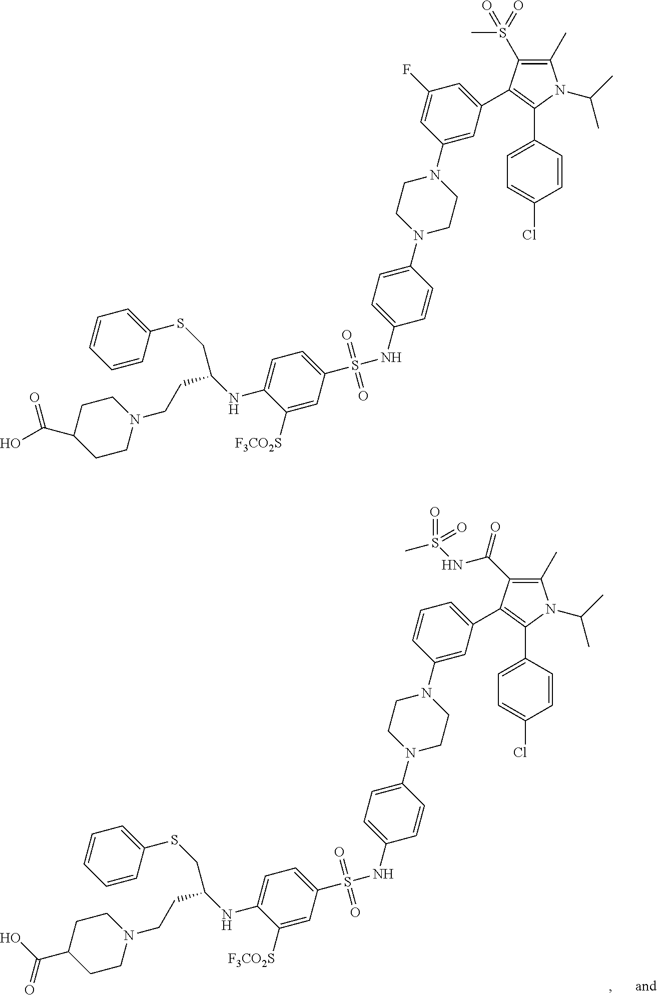

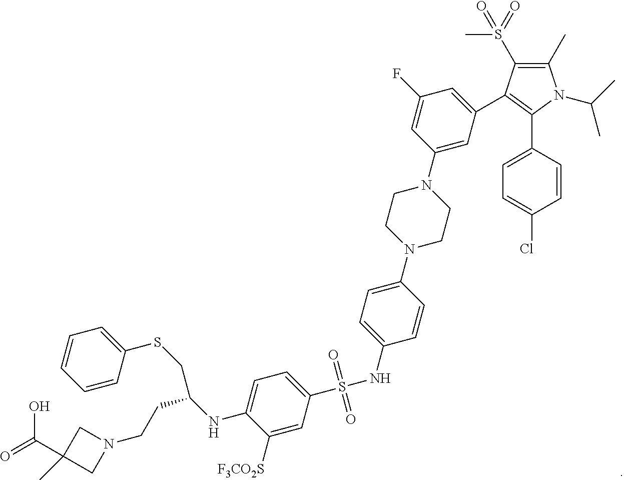

In certain embodiments, the BCL-2/BCL-xL dual inhibitor is (R)-1-(3-(4-(N-(4-(4-(3-(2-(4-chlorophenyl)-1-isopropyl-5-methyl-4-(methylsulfonyl)-1H-pyrrol-3-yl)-5-fluorophenyl) piperazin-1-yl)phenyl) sulfamoyl)-2-(trifluoromethylsulfonyl)phenylamino)-4-(phenylthio)butyl)piperldine-4-carboxylic acid or a pharmaceutically acceptable salt thereof (also referred to as “Compound B” herein).

In certain embodiments, the BCL-2/BCL-xL dual inhibitor or BCL-XL inhibitor or BCL-2 inhibitor described herein has a structural formula (V):

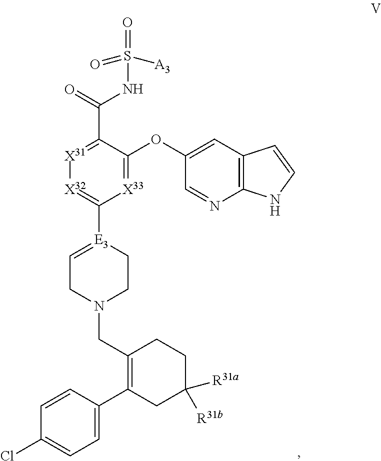

or a pharmaceutically acceptable salt or solvate thereof, wherein:

A₃ is selected from the group consisting of:

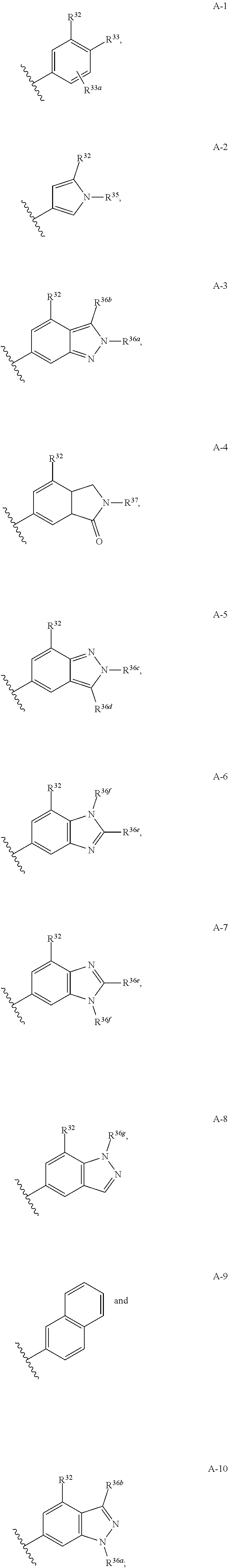

E₃ is a carbon atom and [symbol] is a double bond; or

E₃ is a —C(H)— and [symbol] is a single bond; or

E₃ is a nitrogen atom and [symbol] is a single bond;

X³¹, X³², and X³³ are each independently selected from the group consisting of —CR³⁸ ═ and —N═;

R^{31a} and R^{31b} taken together with the carbon atom to which they are attached form a 3-, 4-, or 5-membered optionally substituted cycloalkyl; or

R^{31a} and R^{31b} taken together with the carbon atom to which they are attached form a 4- or 5-membered optionally substituted heterocyclo;

R³² is selected from the group consisting of —NO₂, —SO₂CH₃, and —SO₂CF₃;

R^{32a} is selected from the group consisting of hydrogen and halogen;

R³³ is selected from the group consisting of hydrogen, —CN, —C≡CH, and —N(R^{34a})(R^{34b});

R^{34a} is selected from the group consisting of optionally substituted C₁₋₆ alkyl, optionally substituted C₃₋₆ cycloalkyl, heterocyclo, heteroalkyl, (cycloalkyl)alkyl, and (heterocyclo)alkyl;

R^{34b} is selected from the group consisting of hydrogen and C₁-4 alkyl;

R³⁵ is selected from the group consisting of is selected from the group consisting of optionally substituted C₁-6 alkyl, heterocyclo, heteroalkyl, (cycloalkyl)alkyl, and (heterocyclo)alkyl;

R^{36a}, R^{36c}, R^{36e}, R^{36f}, and R^{36g} are each independently selected from the group consisting of hydrogen, optionally substituted C₁₋₆ alkyl, optionally substituted C₃₋₆cycloalkyl, optionally substituted aryl, optionally substituted heteroaryl, heterocyclo, heteroalkyl, (cycloalkyl)alkyl, and (heterocyclo)alkyl;

R^{36b} and R^{36d} are each independently selected from the group consisting of hydrogen, C₁₋₄ alkyl, and halogen;

R³⁷ is selected from the group consisting of optionally substituted C₁₋₆ alkyl, heterocyclo, heteroalkyl, (cycloalkyl)alkyl, and (heterocyclo)alkyl; and

R³⁸ is selected from the group consisting of hydrogen and halogen.

In certain embodiments, the BCL-2/BCL-xL dual inhibitor or BCL-XL inhibitor or BCL-2 inhibitor described herein having a structural formula (V) is selected from the group consisting of:

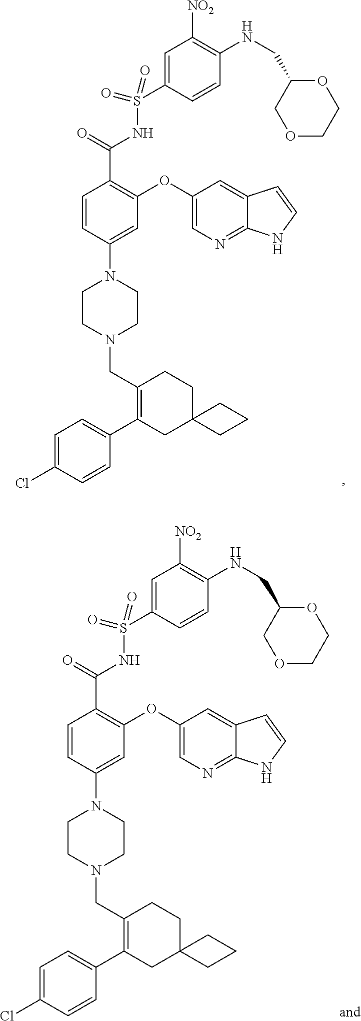 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

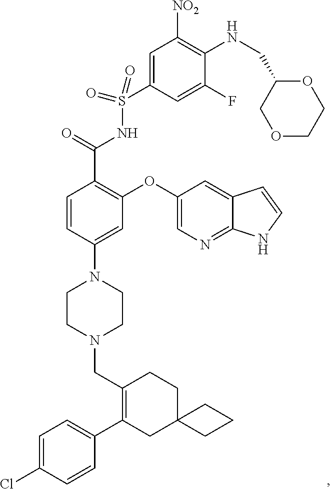 [SMILES: O=C(NS(=O)(=O)c1cc(F)c(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

In certain embodiments, the BCL-xL inhibitor is (S)-N-((4-(((1,4-dioxan-2-yl)methyl)amino)-3-nitrophenyl)sulfonyl)-2-((1H-pyrrolo[2,3-b]pyridin-5-yl)oxy)-4-(4-((6-(4-chlorophenyl)spiro[3.5]non-6-en-7-yl)methyl)piperazin-1-yl)benzamide or a pharmaceutically acceptable salt thereof (also referred to as “Compound C” herein).

Kits

In another aspect, the present disclosure further provides a kit for use in the methods described herein for measuring the level of the at least one biomarker provided herein. In certain embodiments, the kit comprises one or more of reagents, such as the primers, the probes, and/or the antibodies, or microarray provided herein. The primers, the probes, and/or the antibodies may or may not be detectably labeled. In certain embodiments, the kits may further comprise other reagents to perform the methods described herein. In such applications the kits may include any or all of the following: suitable buffers, reagents for isolating nucleic acid, reagents for amplifying the nucleic acid (e.g. polymerase, dNTP mix), reagents for hybridizing the nucleic acid, reagents for sequencing the nucleic acid, reagents for quantifying the nucleic acid (e.g. intercalating agents, detection probes), reagents for isolating the protein, and reagents for detecting the protein (e.g. secondary antibody). Typically, the reagents useful in any of the methods provided herein are contained in a carrier or compartmentalized container. The carrier can be a container or support, in the form of, e.g., bag, box, tube, rack, and is optionally compartmentalized.

In one embodiment, the kit comprises one or more reagent for measuring a level of the complex. In certain embodiments, the reagent comprises a first antibody that can specifically bind to BCL-XL or BCL-2 protein in the complex, and a second antibody that can specifically bind to the BH3-only protein or the BH3-domain containing protein in the complex. In certain embodiments, the first antibody and/or the second antibody is detectably labeled. In certain embodiments, the first antibody is conjugated with a first detectable label, and the second antibody is conjugated with a second detectable label, wherein the first detectable label and the second detectable label can permit generation a detectable signal when in close proximity. In certain embodiments, the first detectable label and the second detectable label both comprise an oligonucleotide.

In certain embodiments, the first antibody is conjugated with a first detectable label, and the second antibody is conjugated with a second detectable label, wherein the first detectable label and the second detectable label can permit generation a detectable signal when in close proximity. In certain embodiments, the first detectable label and the second detectable label comprise a pair of oligonucleotides. When the first antibody and the second antibody are close proximity, the pair of oligonucleotides are capable of interacting to enable enzymatic ligation to provide for a ligated product, which can be amplified to allow detection.

In certain embodiments, one of the first antibody and/or the second antibody is detectably labeled, and the other is capable of being captured.

In certain embodiments, the kit disclosed herein further comprises a second reagent for measuring a level of MCL-1 and/or a third reagent for measuring a level of BCL-XL or BCL-2. In certain embodiments, the second reagent comprises an oligonucleotide capable of hybridizing to the polynucleotide of MCL-1, or an antibody capable of specifically binding to the protein of MCL-1. In certain embodiments, the third reagent comprises an oligonucleotide capable of hybridizing to the polynucleotide of BCL-XL or BCL-2, or an antibody capable of specifically binding to the protein of BCL-XL or BCL-2.

In certain embodiments, the kits can further comprise a standard negative control, and/or a standard positive control.

In addition, the kits may include instructional materials containing directions (i.e., protocols) for the practice of the methods provided herein. While the instructional materials typically comprise written or printed materials they are not limited to such.

In certain embodiments, the kits can further comprise a computer program product stored on a computer readable medium. When computer program product is executed by a computer, it performs the step of comparing the level of the at least one biomarker with a corresponding reference level of the at least one biomarker to determine difference from the reference level. Any medium capable of storing such computer executable instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The computer programs may also be encoded and transmitted using carrier signals adapted for transmission via wired, optical, and/or wireless networks conforming to a variety of protocols, including the Internet. As such, a computer readable medium according to an embodiment of the present invention may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network.

In some embodiments, the present disclosure provides oligonucleotide probes attached to a solid support, such as an array slide or chip, e.g., as described in Eds., Bowtell and Sambrook DNA Microarrays: A Molecular Cloning Manual (2003) Cold Spring Harbor Laboratory Press. Construction of such devices are well known in the art, for example as described in US Patents and Patent Publications U.S. Pat. No. 5,837,832; PCT application WO95/11995; U.S. Pat. Nos. 5,807,522; 7,157,229, 7,083,975, 6,444,175, 6,375,903, 6,315,958, 6,295,153, and 5,143,854, 2007/0037274, 2007/0140906, 2004/0126757, 2004/0110212, 2004/0110211, 2003/0143550, 2003/0003032, and 2002/0041420. Nucleic acid arrays are also reviewed in the following references: Biotechnol Annu Rev (2002) 8:85-101; Sosnowski et al. Psychiatr Genet (2002)12(4): 181-92; Heller, Annu Rev Biomed Eng (2002) 4: 129-53; Kolchinsky et al., Hum. Mutat (2002) 19(4):343-60; and McGail et al., Adv Biochem Eng Biotechnol (2002) 77:21-42.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### Example 1

This example shows that BCL-XL:BIM and BCL-XL:PUMA complex are dominant in patient-derived-xenografts (PDX) models, and a decrease in the BCL-XL:BIM and BCL-XL:PUMA complex indicates the on-target activity, thus be a pharmacodynamics marker for BCL-2 and BCL-XL dual inhibitor and an indicator for the drug efficacy.

BCL-2 has been considered one of the top 10 targets for cancer drug development. The inhibition of BCL-2 and other anti-apoptotic proteins including BCL-XL helps to restore the apoptotic pathway and trigger cancer cell death. Aiming to target much diverse BCL-2 family protein dependence in solid tumors, the inventors have developed a potent BCL-2 and BCL-XL dual inhibitor named as Compound A, which is currently in phase 1 trials for solid tumors. To facilitate the clinical development, the inventors have conducted trials in PDX models derived from solid tumor patients and performed pharmacodynamics (PD) and biomarker studies.

PDX models (including 8 gastric cancers and 3 esophageal cancers) were selected for trials with BCL-2/BCL-XL dual inhibitor Compound A. Models were treated with 100 mg/kg twice weekly dosing schedule for three weeks, and tumor tissues were harvested for complexes analysis (specified in the figure panels) with MSD methods. Pharmacodynamics (PD) and biomarker studies of Compound A treatment were performed by MSD and Western blotting (WB) assays. Specifically, protein extraction was performed with the sample, level of protein complex was measured by MSD methods, and the level of protein was measured by Western Blotting. TGI for each animal was calculated.

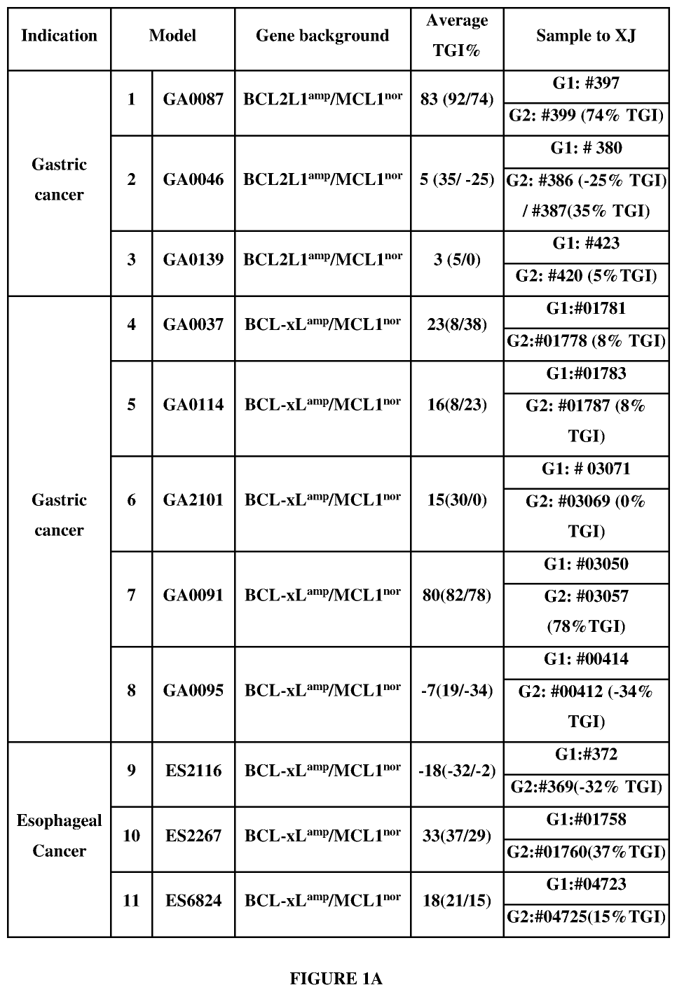

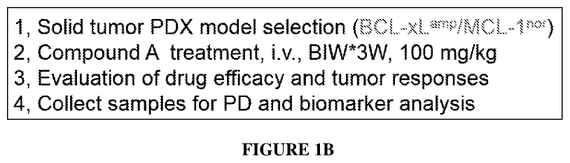

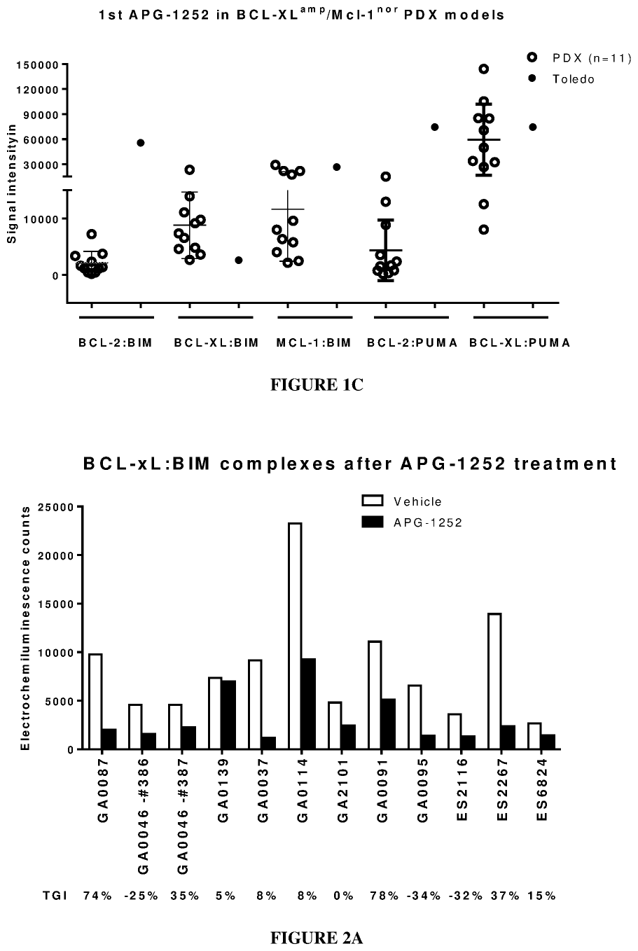

FIGS. 1A-1C illustrate the summary of the in vivo evaluation of Compound A treatment efficiency performed in gastric cancer and esophageal cancer PDS models. FIGS. 1A and 1B shows the summary of the PDX models and the study procedures and results. FIG. 1C shows the baseline levels of different complexes in the PDX models (of solid tumor) and in the Toledo cell line (of hematological cancers), before treatment with Compound A.

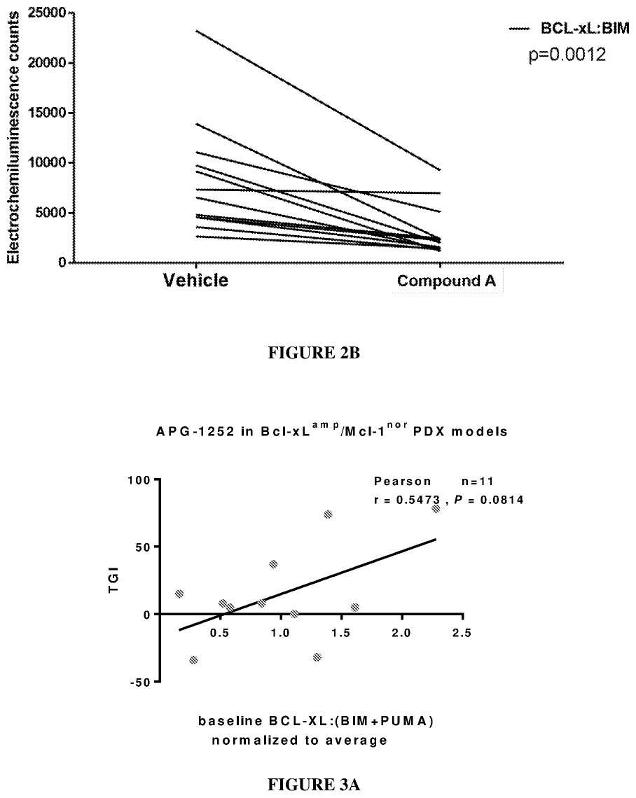

FIGS. 2A and 2B shows that the level of BCL-XL:BIM complex dropped in the PDX models after treatment with Compound A, indicating that Compound A disrupted the BCL-XL:BIM complex in the PDX models.

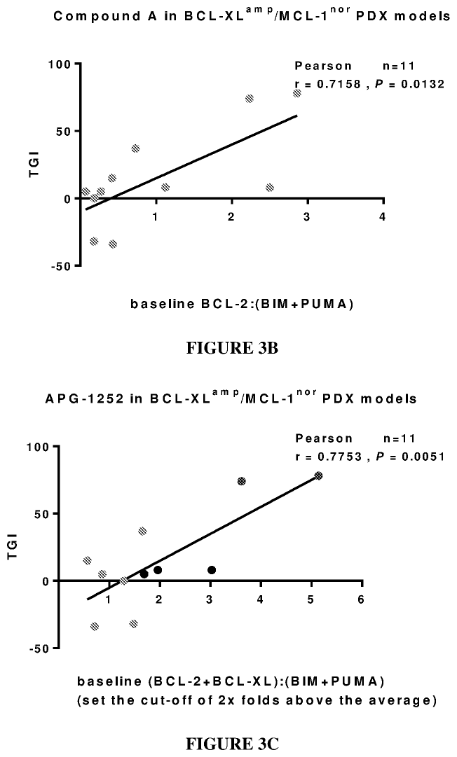

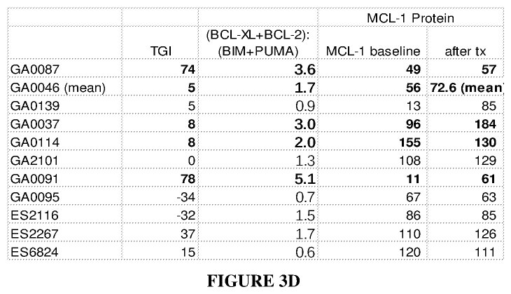

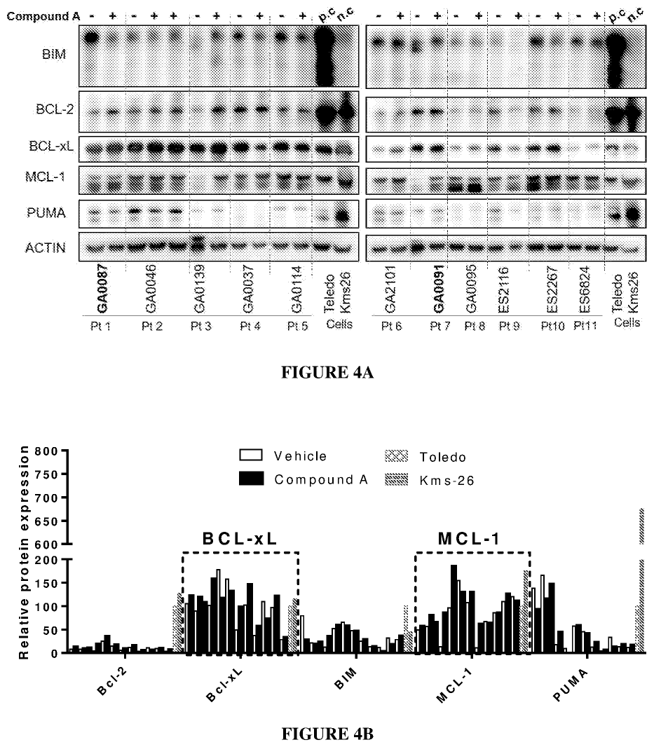

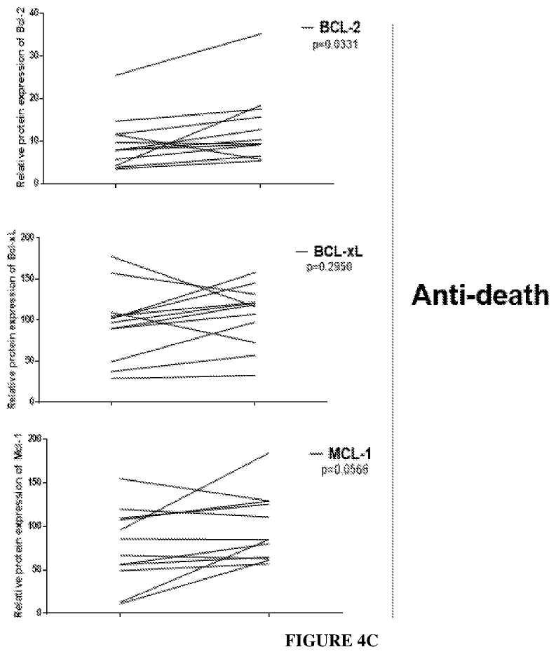

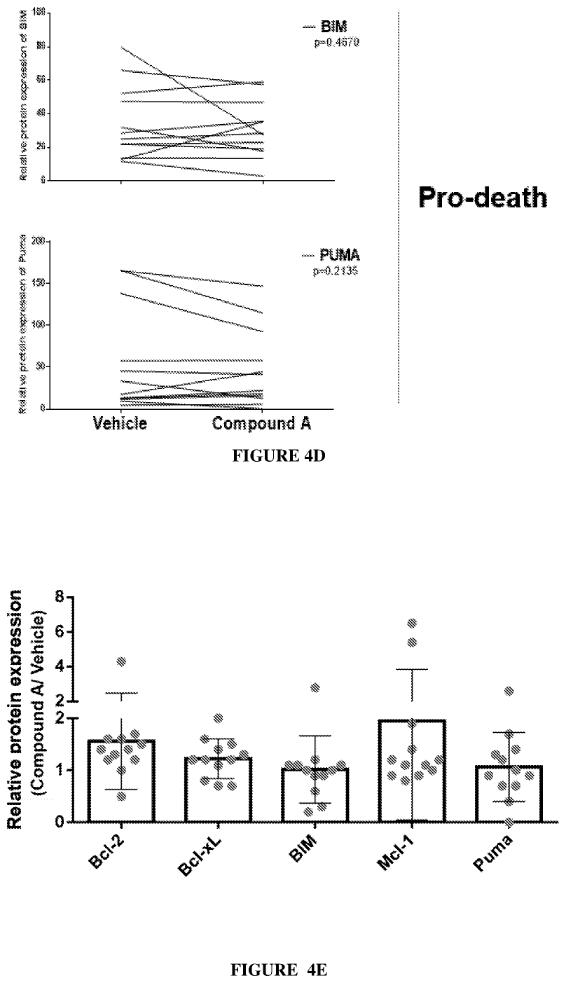

FIG. 3 shows that baseline BCL-2/BCL-xL complex levels correlate with Compound A triggered tumor growth inhibition, which can be used to guide patient selection. FIG. 3A is BCL-XL complexes including BCL-XL:BIM and BCL-XL:PUMA. Baseline complex levels were normalized to the average level of the same group, and plotted with tumor growth inhibition (TGI). Correlation of baseline level of BCL-2 complexes including BCL-2:BIM and BCL-2:PUMA with TGI were shown FIG. 3B. Correlation of baseline levels of BCL-2 complexes and BCL-XL complexes (including BCL-XL:BIM and BCL-XL:PUMA, BCL-2:BIM and BCL-2:PUMA) is shown in FIG. 3C. While BCL-XL or BCL-2 baseline complex alone shown the trend correlating with Compound A triggered tumor growth inhibition, the sum of both complexes predicts the best with r=0.775, and P=0.0051 (FIG. 3C). Besides BCL-XL/BCL-2 complexes, MCL-1 level also affects tumor responses to Compound A, as indicated in the table in FIG. 3D, and FIG. 4. The higher MCL-1 levels (before or after Compound A treatments) lead to the more resistant phenotypes.

FIG. 4A (images) and 4B (quantification) show the western blot results of the cell lysate of the PDX model before and after treatment with Compound A. Further statistical analysis show that Compound A significantly or in trend increases BCL-2 and MCL-1 anti-death protein levels (FIG. 4C), but not pro-death proteins BIM or PUMA (FIG. 4D). FIG. 4E is the plot showing the relative protein expression level change after treatment.

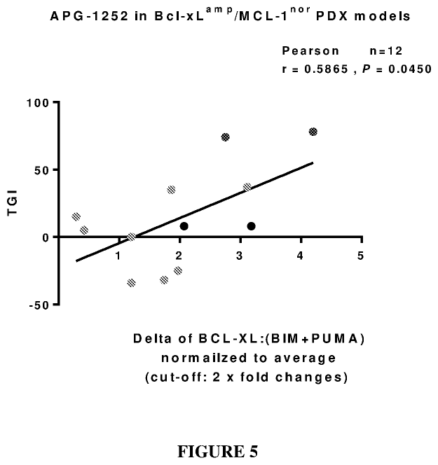

FIG. 5 shows the change of BCL-2/BCL-xL complex level correlates with Compound A triggered tumor growth inhibition, which may be used to predict patient responses and prognosis. PDX tumor samples were subjected to MSD assays for different complexes. BCL-XL complexes including BCL-XL:BIM and BCL-XL:PUMA, and both baseline and post-treated levels were analyzed. The change of BCL-XL complex (namely Delta, the difference between baseline and treated group) was normalized to the average Delta of the same group, and plotted with tumor growth inhibition (TGI). Shown in FIG. 5 is the greater change of the BCL-XL complex, the better TGI. However, the higher MCL-1 levels (blue dots, also in the table in FIG. 3D and FIG. 4) also can affect the overall sensitivity to Compound A.

### Example 2

The inventors further examined whether Compound B, an active metabolite of Compound A used for cell line assays, has similar binding affinity for BCL-2 and BCL-XL, and whether Compound A (or Compound B) is different from ABT-737 or from ABT-263, which are known reference compounds of BCL-2/BCL-XL inhibitor.

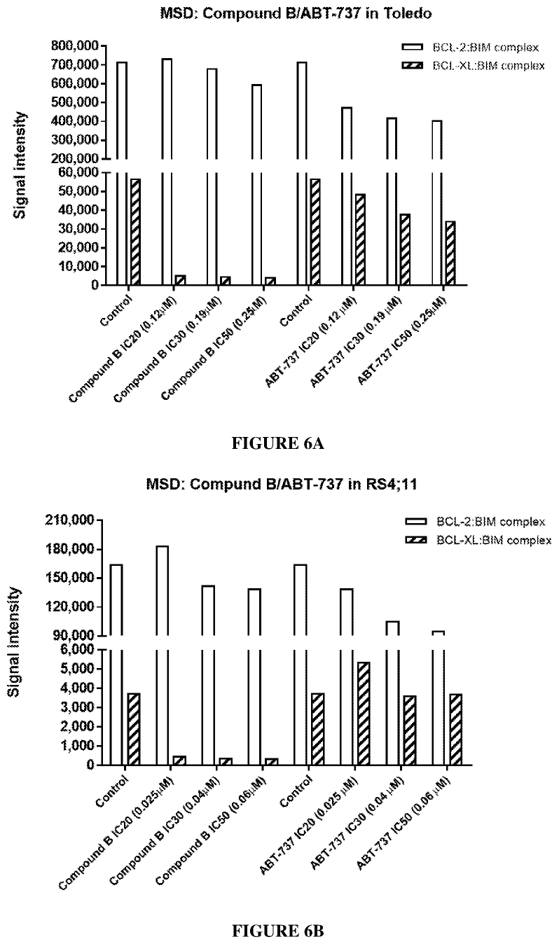

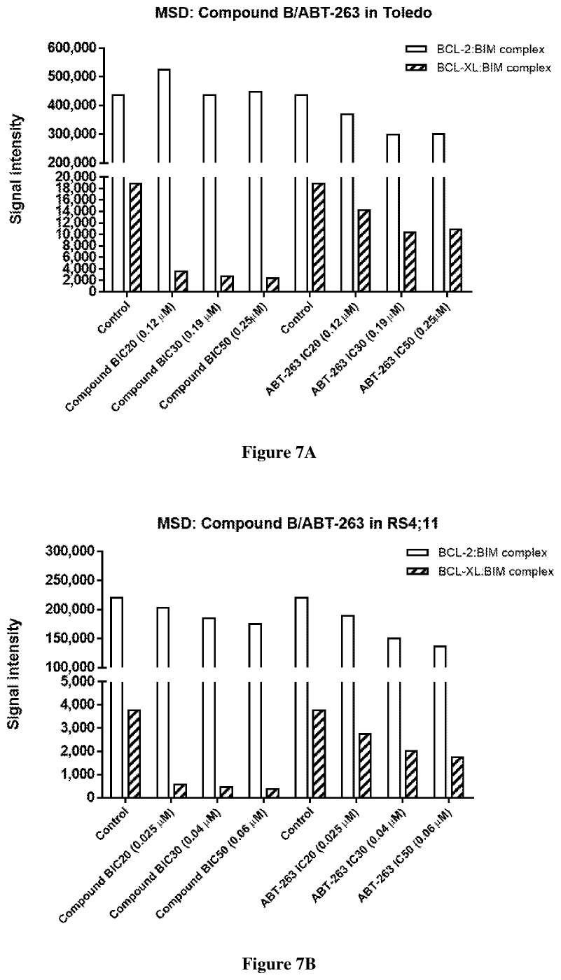

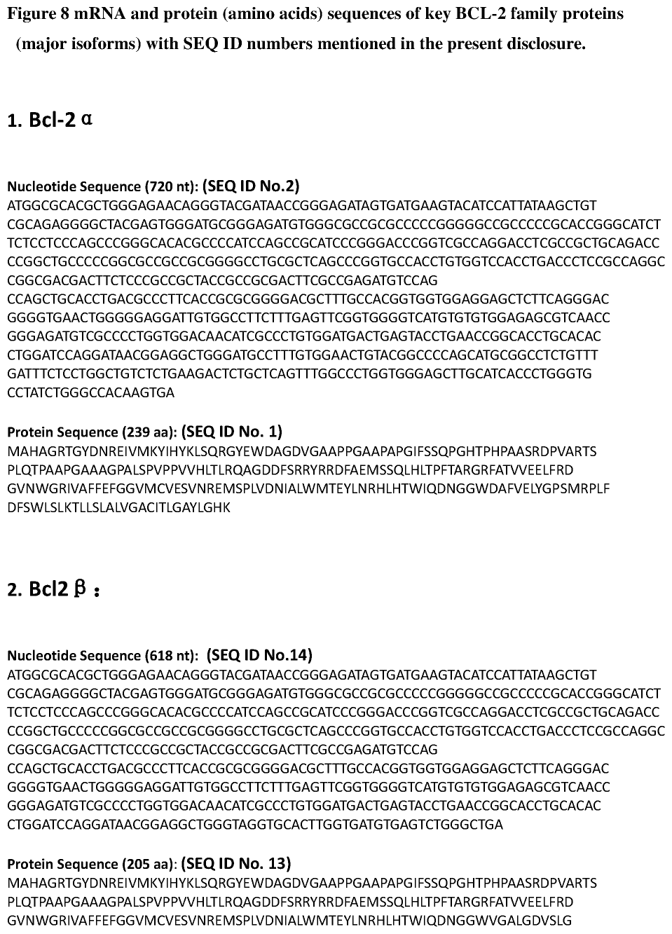

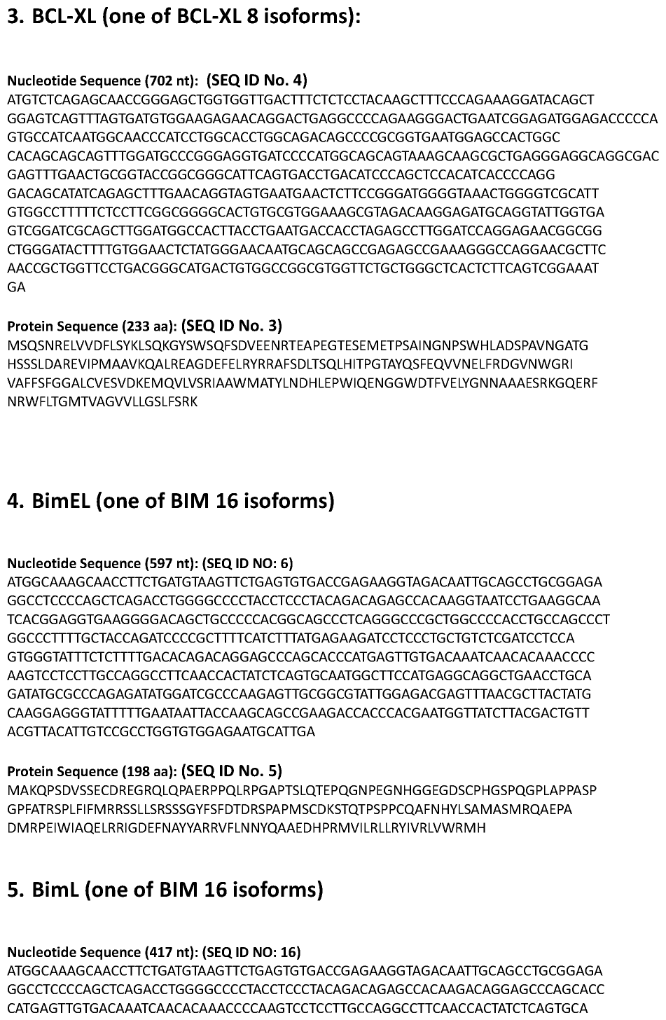

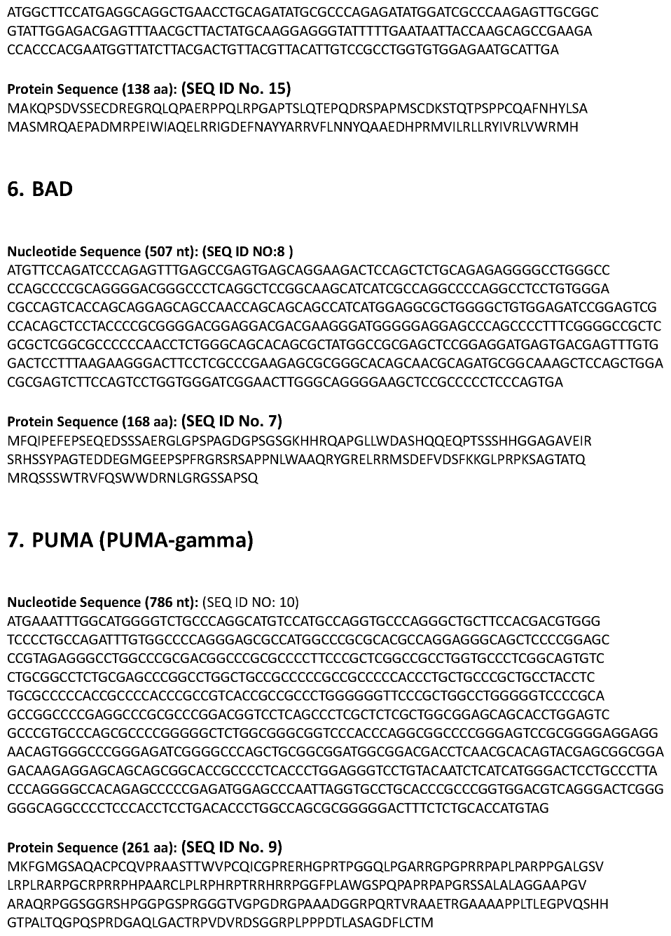

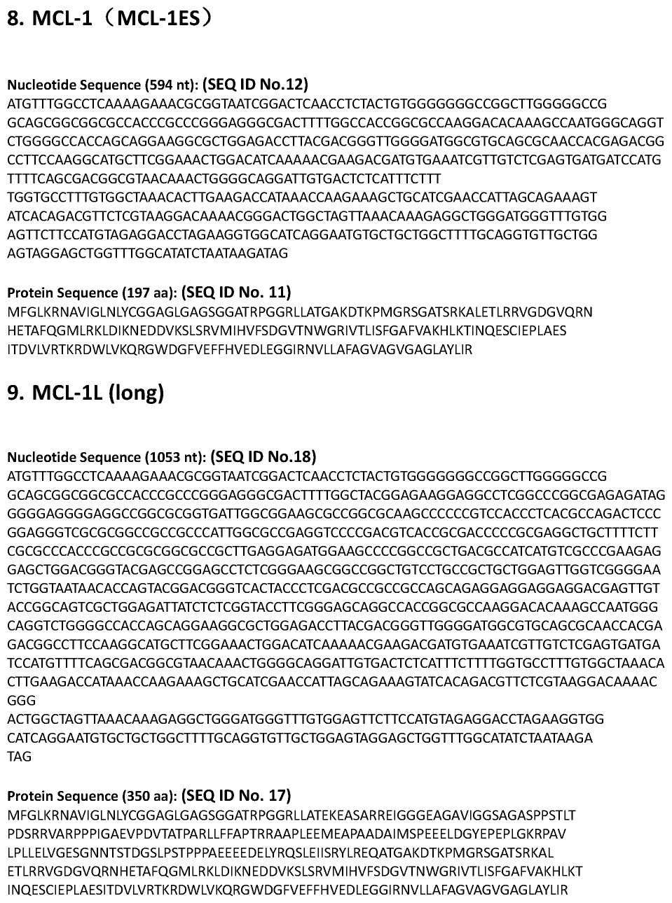

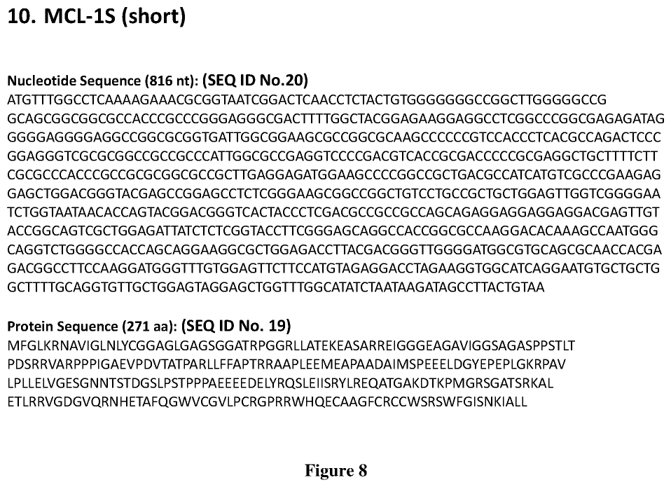

Toledo (a diffuse large B cell lymphoma line) and RS4;11 (an acute lymphocytic leukemia line) were chosen for the study. Cells were treated for 24 hours with compounds specified in FIGS. 6A and 7A (Toledo), and FIGS. 6B and 7B (RS4;11; Compound B, ABT-737 and ABT-263 were used the same concentrations for each cell line) and harvested for complex analysis. Both BCL-2:BIM and BCL-XL:BIM complex were analyzed following previously established MSD advanced ELISA methods.

As shown in FIGS. 6A and 6B, Compound B more potently disrupted BCL-XL:BIM than BCL-2:BIM complex in Toledo and RS4;11 cell lines. In comparison, ABT-737, while also being a BCL-2/BCL-XL dual inhibitor (but structurally different from Compound B), showed much less disruption of BCL-XL:BIM complex than Compound B in Toledo cells (see FIG. 6A), and almost no disruption of BCL-xL:BIM complex in RS4 cells (see FIG. 6B).

Similarly, as shown in FIGS. 7A and 7B, Compound B more potently disrupted BCL-XL:BIM than BCL-2:BIM complex in Toledo and RS4;11 cell lines. In comparison, ABT-263, while also being a BCL-2/BCL-XL dual inhibitor (but structurally different from Compound B), showed much less disruption of BCL-XL:BIM complex than Compound B in Toledo cells (see FIG. 7A) and in RS4;11 cells (see FIG. 7B). In contrast, ABT263 showed reduction in BCL-2:BIM complex.

In light of results in FIG. 1C, where solid tumor cells are shown to have much higher level of BCL-XL:BIM complex than blood cancer cells (e.g. Toledo cells), disruption of BCL-XL:BIM complex would be important for treating solid tumor. These blood cell line results are consistent with complex analysis from gastric/esophageal cancer PDX trials illustrated in EXAMPLE 1, confirming that Compound A (in PDX) or Compound B (in cell line) is better in targeting BCL-XL than BCL-2 complex, thus is distinct from the reference compound ABT-737 or ABT-263. This results also indicate that the level of the BCL-XL:BIM complex can be a biomarker for predicating responsiveness to BCL-2/BCL-XL dual inhibitors such as Compound A and Compound B.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

What is claimed is:

1. A method for treating cancer in a subject in need thereof, the method comprising:
a) measuring a test level of at least one biomarker comprising a first complex comprising BCL-XL or BCL-2 protein, in a test sample comprising a cell obtained from the subject;
b) comparing the test level of the at least one biomarker with a corresponding reference level of the at least one biomarker to determine a difference; and
c) administering a BCL-2/BCL-XL dual inhibitor or a BCL-XL inhibitor or a BCL-2 inhibitor to the subject when the difference reaches a threshold.

2. A method for identifying and/or selecting a subject having cancer for treatment with a BCL-2/BCL-XL dual inhibitor or a BCL-XL or BCL-2 inhibitor, the method comprising:
a) measuring a test level of at least one biomarker comprising a first complex comprising BCL-XL or BCL-2 protein, in a test sample comprising a cell obtained from the subject;
b) comparing the test level of the at least one biomarker with a corresponding reference level of the at least one biomarker to determine a difference; and
c) determining that the subject is likely to respond to the treatment with the BCL-2/BCL-XL dual inhibitor or the BCL-XL inhibitor or the BCL-2 inhibitor when the difference reaches a threshold.

3. A method for monitoring therapeutic efficacy in a subject having cancer and having been treated with a BCL-2/BCL-XL dual inhibitor or a BCL-XL or BCL-2 inhibitor for a therapeutic period, the method comprising:
a) obtaining a test sample comprising a cell from the subject after the therapeutic period;
b) measuring a level of at least one biomarker comprising a first complex comprising BCL-X1 or BCL-2 in the test sample to obtain a post-treatment level of the at least one biomarker;
c) comparing the post-treatment level with a baseline level of the at least one biomarker measured on a test sample obtained from the subject before the therapeutic period, to determine post-treatment change in the level of the at least one biomarker; and
d) continuing administering the BCL-2/BCL-XL dual inhibitor or the BCL-XL or the BCL-2 inhibitor to the subject when the post-treatment change reaches a threshold, or when the post-treatment change does not reach the threshold, increasing the dose or the dosing frequency of the BCL-2/BCL-XL dual inhibitor or the BCL-XL inhibitor or the BCL-2 inhibitor to the subject, administering a second anti-cancer therapeutic agent in combination to the BCL-2/BCL-XL dual inhibitor or the BCL-XL inhibitor or the BCL-2 inhibitor to the subject, or discontinuing administering the BCL-2/BCL-XL dual inhibitor or the BCL-XL inhibitor or the BCL-2 inhibitor to the subject.

4. The method of any of claims 1-3, wherein the at least one biomarker further comprises a second complex comprising BCL-XL or BCL-2 protein.

5. The method of any of claims 1-4, wherein the first and/or the second complex comprises BCL-XL protein complexed with a BH3-only protein, BCL-2 protein complexed with a BH3-only protein, BCL-XL protein complexed with a BH3-containing protein, or BCL-2 protein complexed with a BH3-containing protein.

6. The method of claim 5, wherein the BH3-only protein is selected from the group consisting of: BIM, BID, BAD, BIK, HRK, BMF, and PUMA.

7. The method of claim 5, wherein the BH3-containing protein is BAX or BAK.

8. The method of any of claims 1-7, wherein the at least one biomarker comprises two or more complexes selected from the group consisting of: BCL-XL:BIM, BCL-XL:PUMA, BCL-2:BIM, BCL-2:PUMA, MCL-1:BIM, MCL-1:PUMA, and any combination thereof.

9. The method of any of claims 1-8, wherein the level of the at least one biomarker comprises combination of the level of the first complex and the level of the second complex.

10. The method of any of claims 1-9, wherein the level of the first and/or the second complex is measured by a protein-protein interaction assay.

11. The method of claim 9, wherein the protein-protein interaction assay is based on immunoassay or proximity assays.

12. The method of claim 10 or 11, wherein the protein-protein interaction assay is meso scale discovery (MSD) advanced enzyme-linked immunosorbent assay (MSD-ELISA), standard complex ELISA, proximity ligation assay, co-immunoprecipitation, immunoblotting assay, or cross-linking assay.

13. The method of any of claims 1-12, wherein the level of the first and/or the second complex is measured by using an antibody that specifically bind to the complex or to the BCL-XL protein or to the BCL-2 protein.

14. The method of any of claims 1-13, wherein the first and/or the second complex is a dominant complex in the sample.

15. The method of any of claims 1-14, wherein the at least one biomarker further comprises MCL-1.

16. The method of any of claims 1-15, wherein the at least one biomarker further comprises BCL-2 or BCL-XL.

17. The method of claim 15 or 16, wherein the level of MCL-1, BCL-2 or BCL-XL is measured at mRNA level, protein level or DNA level.

18. The method of claim 17, wherein the level of MCL-1, BCL-2 or BCL-XL is measured by an amplification assay, a hybridization assay, a sequencing assay, an immunoassay, a spectrometry method, or a proximity assay.

19. The method of any of claims 1-18, wherein the cancer is a solid tumor.

20. The method of claim 19, wherein the solid tumor is lung cancer, gastric cancer, esophageal cancer, colon cancer, cholangiocarcinoma, liver cancer, breast cancer, cervical cancer, ovarian cancer, head and neck cancer or brain tumors.

21. The method of claim 19 or 20, wherein the at least one biomarker comprises BCL-XL:BIM and BCL-XL:PUMA.

22. The method of any of claims 1-18, wherein the cancer is a blood cancer.

23. The method of claim 21, wherein the blood cancer is chronic lymphocytic leukemia (CLL), acute myeloid leukemia (AML), multiple myeloma (MM), Waldenstrom macroglobulinemia (WM), acute lymphoblastic leukemia (ALL) or lymphoma.

24. The method of claim 22 or 23, wherein the at least one biomarker comprises BCL-2:BIM and BCL-2:PUMA.

25. The method of any of claims 1-24, wherein the reference level is an average level of the at least one biomarker in representative samples of the same type of cancer.

26. The method of any of claims 1-24, wherein the reference level is an empirical level of the biomarker in a tumor sample of the same type or in a certain type of cancer (e.g. in blood cancer) or in general cancer.

27. The method of any of claims 1-26, wherein the comparing is performed with an algorithm.

28. The method of claim 27, wherein the algorithm is a classification algorithm.

29. The method of claim 28, wherein the difference comprises a difference in a test score for the test level and a reference score for the reference level, and wherein the test and the reference score are calculated by the algorithm.

30. The method of any of claims 1-29, wherein the at least one biomarker comprises a combination of BCL-XL:BIM and BCL-XL:PUMA; or a combination of BCL-2:BIM and BCL-2:PUMA.

31. The method of any of claims 1-30, wherein the at least one biomarker comprises a combination of BCL-2:BIM, BCL-2:PUMA, BCL-XL:BIM, and BCL-XL:PUMA.

32. The method of claim 31, wherein the threshold is reached when the test level of the combination of BCL-2:BIM, BCL-2:PUMA, BCL-XL:BIM, and BCL-XL:PUMA is at least 2-fold above the reference level, or wherein the threshold is reached when the post-treatment change is at least 2-fold reduction.

33. The method of any of claims 15-32, wherein the threshold is reached when the test level of MCL-1 is no more than 100% of the reference level of MCL-1.

34. The method of any of claims 16-33, wherein the threshold is reached when the test level of BCL-2 or BCL-XL is at least 2-fold of the reference level of BCL-2 or BCL-XL.

35. The method of any of claims 1-34, wherein the test sample is a bodily fluid sample or a tissue sample.

36. The method of any of preceding claims, wherein the BCL-2/BCL-XL dual inhibitor has a structure of formula (I), formula (II), or formula (III): 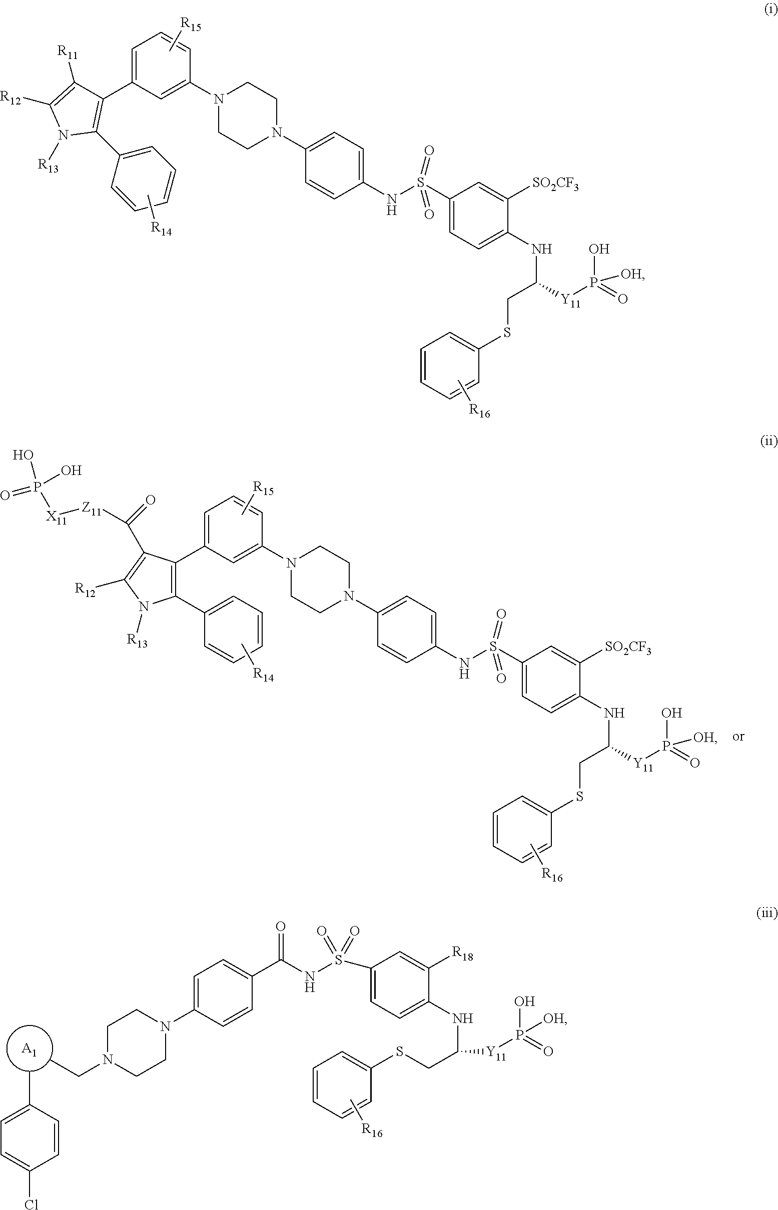
wherein the A₁ ring is 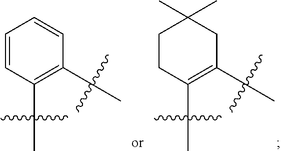
X₁₁, substituted or unsubstituted, is selected from the group consisting of alkylene, alkenylene, cycloalkylene, cycloalkenylene, and heterocycloalkylene;
Y₁₁ is selected from the group consisting of (CH₂)ₙ—N(R₁₁ₐ) and 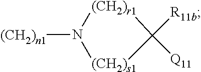
Q₁₁ is selected from the group consisting of O, O(CH₂)₁₋₃, NR_{11c}, NR_{11c} (C₁₋₃alkylene), OC(═O)(C₁₋₃alkylene), C(═O)O, C(═O)O(C₁₋₃alkylene), NHC(═O)(C₁₋₃alkylene), C(═O)NH, and C(═O)NH(C₁₋₃alkylene);
Z₁₁ is O or NR_{11c}
R₁₁ and R₁₂, independently, are selected from the group consisting of H, CN, NO₂, halo, alkyl, cycloalkyl, alkenyl, cycloalkenyl, alkynyl, aryl, heteroaryl, heterocycloalkyl, OR₁′, SR₁′, NR₁′R₁″, COR₁′, CO₂R₁′, OCOR₁′, CONR₁′R₁″, CONR₁′SO₂R₁″, NR₁“COR₁”, NR₁′CONR₁″R₁′″, NR₁′C═SNR₁″R₁′″, NR₁'SO₂R₁″, SO₂R₁′, and SO₂NR₁′R₁″;
R₁₃ is selected from a group consisting of H, alkyl, cycloalkyl, alkenyl, cycloalkenyl, alkynyl, aryl, heteroaryl, heterocycloalkyl, OR₁′, NR₁′R₁″, OCOR₁′, CO₂R₁′, COR₁′, CONR₁′R₁″, CONR₁′SO₂R₁″, C₁₋₃alkyleneCH(OH)CH₂OH, SO₂R₁′, and SO₂NR₁′R₁″;
R₁′, R₁″, and R₁′″, independently, are H, alkyl, cycloalkyl, alkenyl, cycloalkenyl, alkynyl, aryl, heteroaryl, C₁-3alkyleneheterocycloalkyl, or heterocycloalkyl;
R₁′ and R₁″, or R₁″ and R₁′″, can be taken together with the atom to which they are bound to form a 3 to 7 membered ring;
R₁₄ is hydrogen, halo, C₁₋₃alkyl, CF₃, or CN;
R₁₅ is hydrogen, halo, C₁₋₃alkyl, substituted C₁₋₃alkyl, hydroxyalkyl, alkoxy, or substituted alkoxy;
R₁₆ is selected from the group consisting of H, CN, NO₂, halo, alkyl, cycloalkyl, alkenyl, cycloalkenyl, alkynyl, aryl, heteroaryl, heterocycloalkyl, OR₁′, SR₁′, NR₁′R₁″, CO₂R₁′, OCOR₁′, CONR₁′R₁″, CONR₁“SO₂R₁″, NR₁′COR₁″, NR₁′CONR₁″R₁′″, NR₁′C═SNR₁″R₁′″, NR₁'SO₂R₁″, SO₂R₁′, and SO₂NR₁′R₁^{”};
R₁₇, substituted or unsubstituted, is selected form the group consisting of hydrogen, alkyl, alkenyl, (CH₂)₀₋₃cycloalkyl, (CH₂)₀₋₃cycloalkenyl, (CH₂)₀₋₃heterocycloalkyl, (CH₂)₀₋₃aryl, and (CH₂)₀₋₃heteroaryl;
R₁₈ is selected form the group consisting of hydrogen, halo, NO₂, CN, CF₃SO₂, and CF₃;
R₁₁ₐ is selected from the group consisting of hydrogen, alkyl, heteroalkyl, alkenyl, hydroxyalkyl, alkoxy, substituted alkoxy, cycloalkyl, cycloalkenyl, and heterocycloalkyl;
R_{11b} is hydrogen or alkyl;
R_{11c} is selected from the group consisting of hydrogen, alkyl, substituted alkyl, hydroxyalkyl, alkoxy, and substituted alkoxy; and
n₁, r₁, and s₁, independently, are 1, 2, 3, 4, 5, or 6;
or a pharmaceutically acceptable salt of (I), (II), or (III).

37. The method of any of preceding claims, wherein the BCL-2/BCL-XL dual inhibitor has a structure of formula (IV): 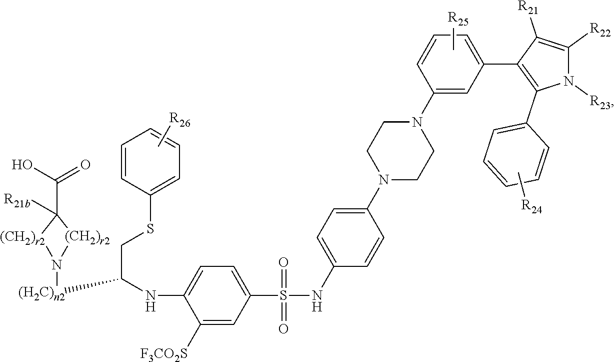
wherein,
R₂₁ is SO₂R₂′,
R₂₂ is alkyl, preferably C₁-C₄ alkyl, more preferably methyl, propyl, or isopropyl,
R₂₃ is alkyl, preferably C₁-C₄ alkyl, more preferably methyl, propyl, or isopropyl,
R₂₄ is halogen, preferably fluoride, chloride,
R₂₅ is halogen, preferably fluoride, chloride,
R₂₆ is selected from H, halogen, alkyl, preferably fluoride, chloride, C1-C4 alkyl, more preferably methyl, propyl, isopropyl
R_{21b} is H or alkyl, preferably C1-C4 alkyl, more preferably methyl, propyl, or isopropyl,
n₂, r₂ and s₂ are independently 1, 2, 3, 4, 5 or 6, more preferably, r₂ and s₂ are both 2 and n₂ is 3, 4 or 5, more preferably, all of n₂, r₂ and s₂ are 2, and
R₂′ is alkyl, preferably C1-C4 alkyl, more preferably methyl, propyl, or isopropyl.

38. The method of any of preceding claims, wherein the BCL-2/BCL-XL dual inhibitor has a structure of formula (V): 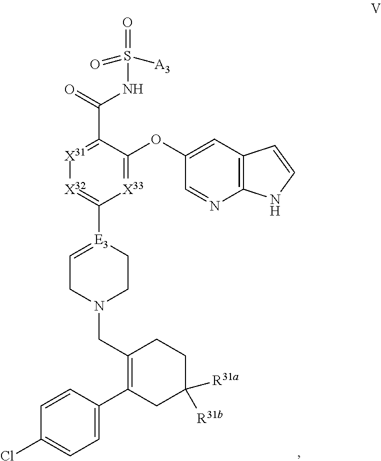
or a pharmaceutically acceptable salt or solvate thereof, wherein:
A₃ is selected from the group consisting of: 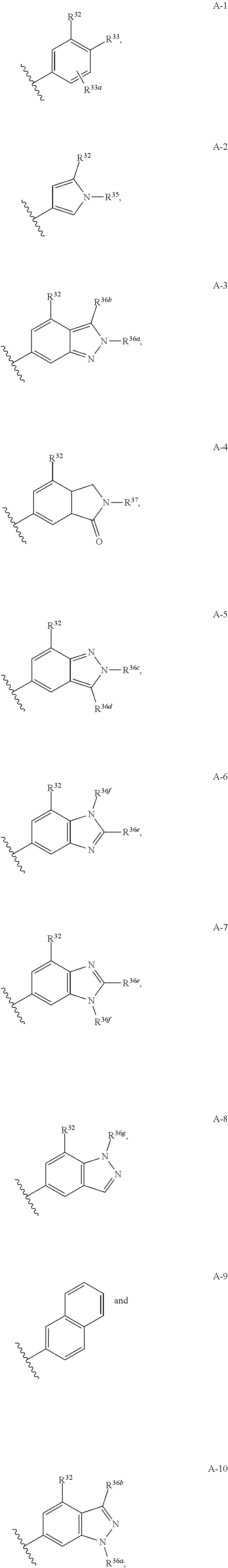
E₃ is a carbon atom and [symbol] is a double bond; or
E₃ is a —C(H)— and [symbol] is a single bond; or
E₃ is a nitrogen atom and [symbol] is a single bond;
X³¹, X³², and X³³ are each independently selected from the group consisting of —CR³⁸═ and —N═;
R^{31a} and R^{31b} taken together with the carbon atom to which they are attached form a 3-, 4-, or 5-membered optionally substituted cycloalkyl; or
R^{31a} and R^{31b} taken together with the carbon atom to which they are attached form a 4- or 5-membered optionally substituted heterocyclo;
R³² is selected from the group consisting of —NO₂, —SO₂CH₃, and —SO₂CF₃;
R^{32a} is selected from the group consisting of hydrogen and halogen;
R³³ is selected from the group consisting of hydrogen, —CN, —C≡CH, and —N(R^{34a})(R^{34b});
R^{34a} is selected from the group consisting of optionally substituted C₁₋₆ alkyl, optionally substituted C₃₋₆ cycloalkyl, heterocyclo, heteroalkyl, (cycloalkyl)alkyl, and (heterocyclo)alkyl;
R^{34b} is selected from the group consisting of hydrogen and C₁₋₄ alkyl;
R³⁵ is selected from the group consisting of is selected from the group consisting of optionally substituted C₁₋₆ alkyl, heterocyclo, heteroalkyl, (cycloalkyl)alkyl, and (heterocyclo)alkyl;
R^{36a}, R^{36c}, R^{36e}, R^{36f}, and R^{36g} are each independently selected from the group consisting of hydrogen, optionally substituted C₁₋₆ alkyl, optionally substituted C₃₋₆cycloalkyl, optionally substituted aryl, optionally substituted heteroaryl, heterocyclo, heteroalkyl, (cycloalkyl)alkyl, and (heterocyclo)alkyl;
R^{36b} and R^{36d} are each independently selected from the group consisting of hydrogen, C₁₋₄ alkyl, and halogen;
R³⁷ is selected from the group consisting of optionally substituted C₁₋₆ alkyl, heterocyclo, heteroalkyl, (cycloalkyl)alkyl, and (heterocyclo)alkyl; and
R³⁸ is selected from the group consisting of hydrogen and halogen.

39. The method of any of the preceding claims, wherein the BCL-2/BCL-xL dual inhibitor or BCL-XL inhibitor or BCL-2 inhibitor is selected from the group consisting of:

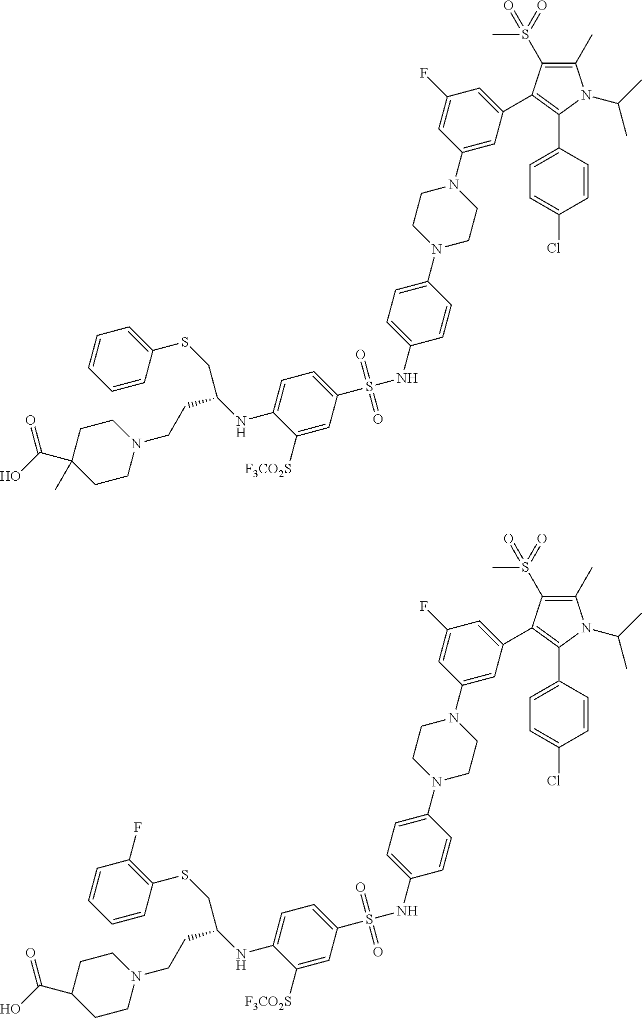

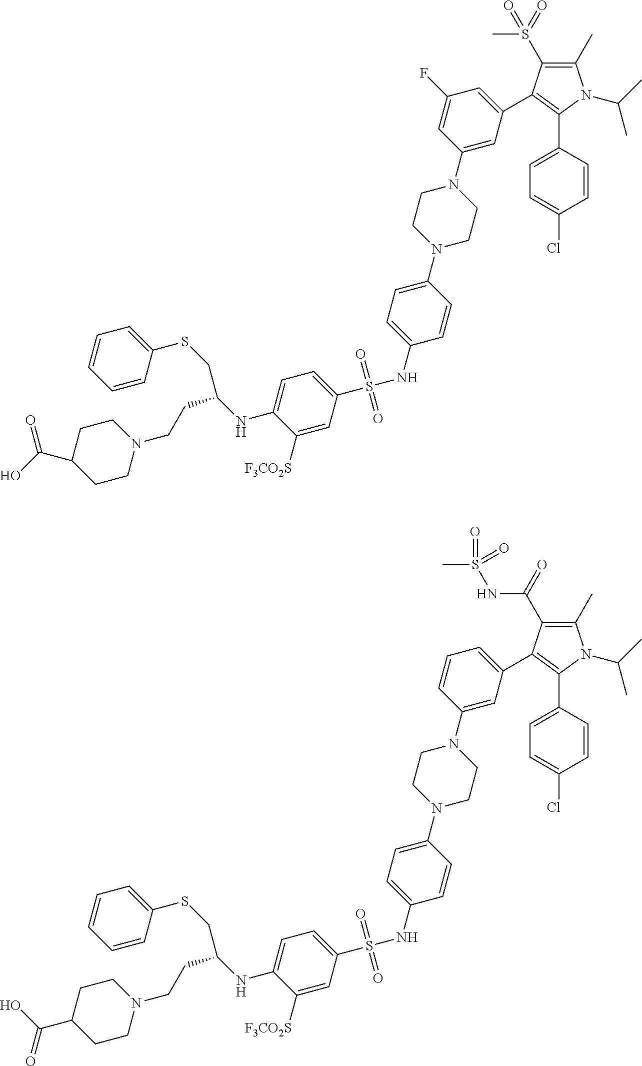

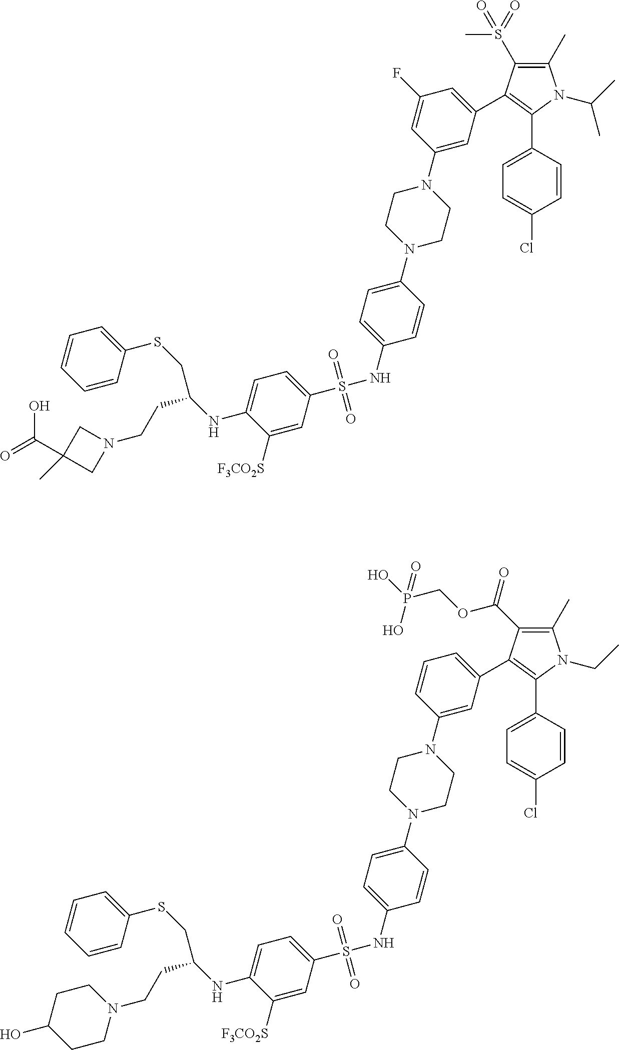

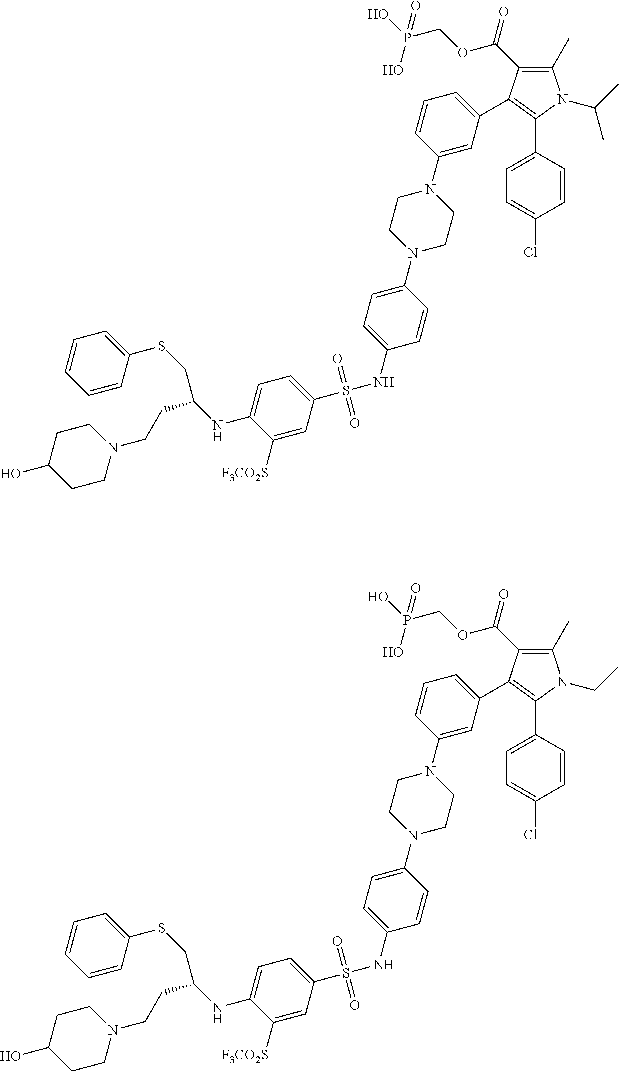

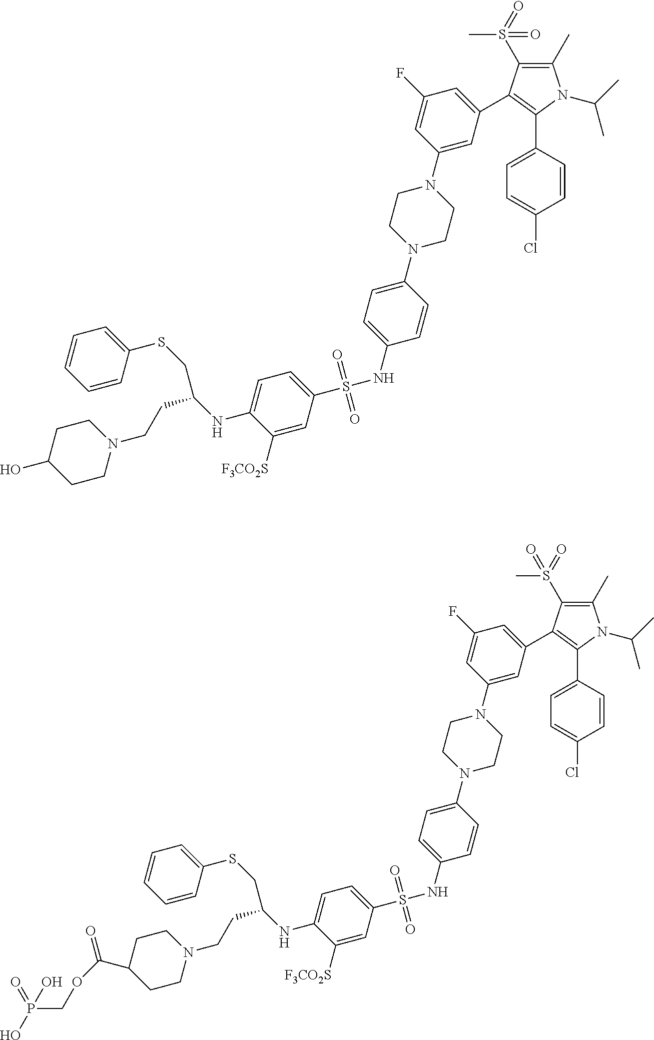

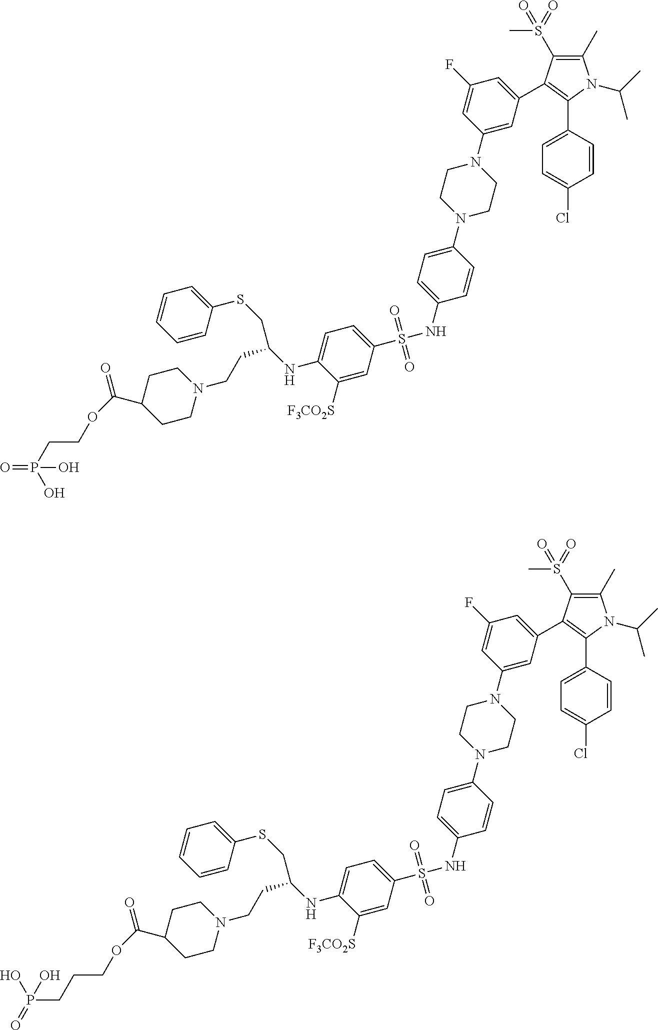

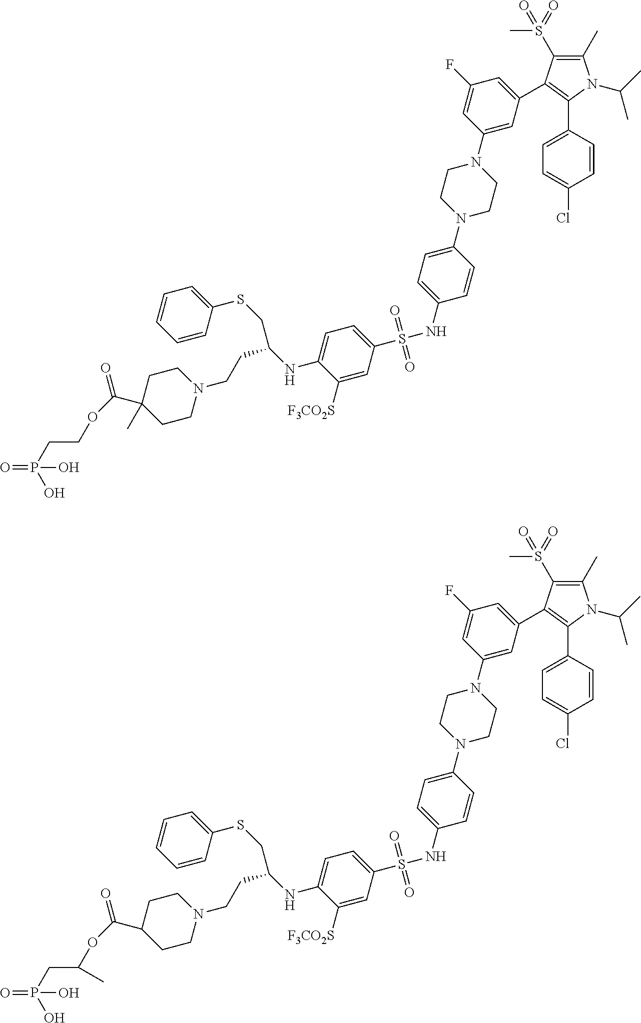

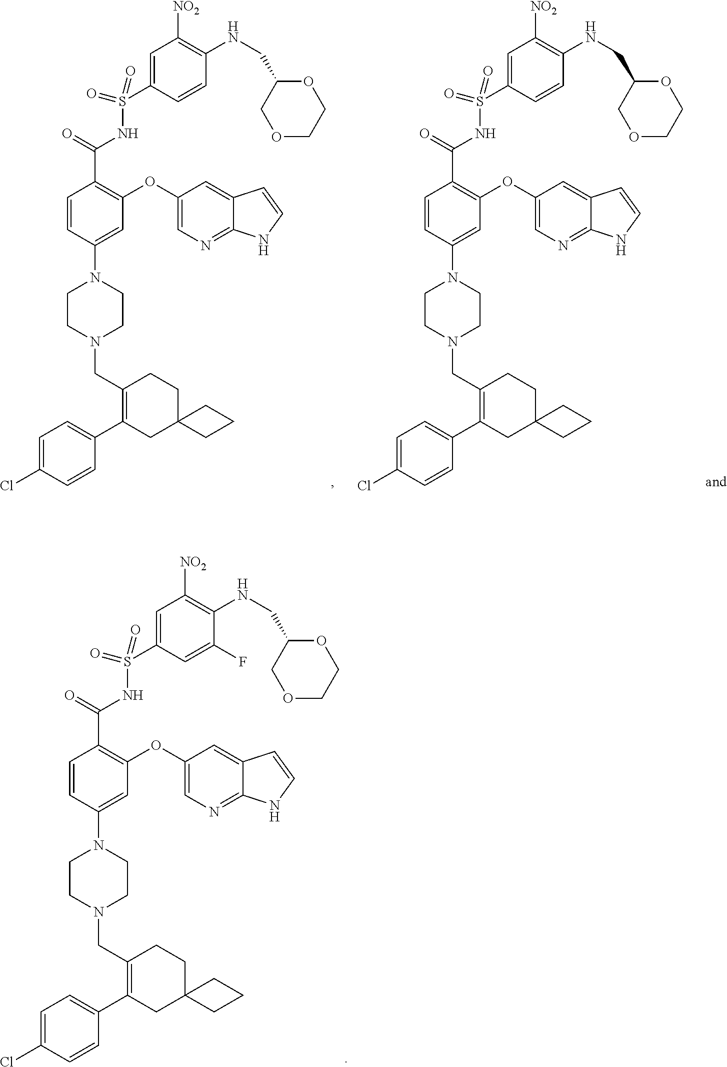 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1cc(F)c(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

40. A kit for use in the method according to any one of claims 1-39, comprising at least one reagent for measuring a level of the at least one biomarker.

41. The kit of claim 40, wherein the at least one reagent comprises a first reagent comprising a first antibody that specifically binds to the complex or to the BCL-XL protein, or to the BCL-2protein.

42. The kit of claim 41, wherein the at least one reagent further comprises a second reagent comprising a second antibody that specifically binds to the BH3-only protein or the BH3-domain containing protein in the complex.

43. The kit of any of claim 41 or 42, wherein the first antibody and/or the second antibody is detectably labeled.

44. The kit of any of claim 41 or 42, wherein one of the first antibody and/or the second antibody is detectably labeled, and the other is capable of being captured.

45. The kit of any of claim 40 or 44, wherein the at least one reagent further comprises a third reagent comprising a first oligonucleotide capable of hybridizing to the polynucleotide of MCL-1, or a third antibody capable of specifically binding to the protein of MCL-1.

46. The kit of any of claim 40 or 45, wherein the at least one reagent further a fourth reagent comprising a second oligonucleotide capable of hybridizing to the polynucleotide of BCL-XL or BCL-2, or a fourth antibody capable of specifically binding to the protein of BCL-XL or BCL-2.

47. Use of at least one reagent for measuring a level of at least one biomarker comprising a complex comprising BCL-XL protein or BCL-2 protein in the manufacture of a kit for performing the method according to any one of claims 1-39.
